(19) United States
(12) Patent Application Publication
(10) Pub. No.: US 2008/0045498 A1
(43) Pub. Date: Feb. 21, 2008

(54) POLYCYCLIC VIRAL INHIBITORS

(75) Inventors: Ronald Conrad Griffith, Escondido, CA (US); Christopher Don Roberts, Belmont, CA (US); Franz Ulrich Schmitz, Mill Valley, CA (US); Janos Botyanszki, Fremont, CA (US); Rachel Elizabeth Marek, Austin, TX (US); Dong-Fang Shi, Fremont, CA (US); Son Minh Pham, San Francisco, CA (US)

Correspondence Address: FOLEY & LARDNER LLP, 975 PAGE MILL ROAD, PALO ALTO, CA, 94304, US

(73) Assignee: Genelabs Technologies, Inc.

(21) Appl. No.: 11/780,378

(22) Filed: Jul. 19, 2007

Related U.S. Application Data
(60) Provisional application No. 60/832,520, filed on Jul. 20, 2006.

(51) Int. Cl. A61K 31/55; A61K 31/5377; A61K 31/5513; A61K 31/553; A61P 31/12; C07D 471/04; C07D 471/22; C07D 487/04; C07D 495/22; C07D 498/04

(52) U.S. Cl. 514/211.09; 514/214.01; 514/219; 514/233.2; 514/280; 540/471; 540/546; 540/555; 540/577; 544/125

(57) ABSTRACT

Disclosed are compounds and compositions of Formula (A) and their uses for treating Flaviviridae family virus infections.

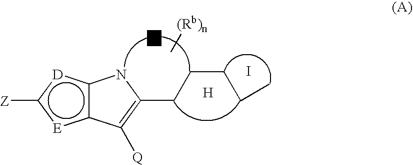

### CROSS-REFERENCE TO RELATED APPLICATION

This application claims priority to co-pending provisional application U.S. Ser. No. 60/832,520 filed on Jul. 20, 2006, which is incorporated herein by reference in its entirety.

### BACKGROUND OF THE INVENTION

1. Field of the Invention

The invention relates to the field of pharmaceutical chemistry, in particular to compounds, compositions and methods for treating viral infections in mammals mediated, at least in part, by a virus in the Flaviviridae family of viruses.

### REFERENCES

The following publications are cited in this application as superscript numbers:
1. Szabo, E. et al., Pathol. Oncol. Res. 2003, 9:215-221.
2. Hoofnagle J. H., Hepatology 1997, 26:15 S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem. 39, 223-237, 2004.
9. Watashi, K. et al., Molecular Cell, 19, 111-122, 2005
10. Horsmans, Y. et al., Hepatology, 42, 724-731, 2005

2. State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of 3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

The present invention is directed to novel compounds, compositions, and methods for treating of viral infections in mammals mediated, at least in part, by a member of the Flaviviridae family viruses such as HCV. In one embodiment, provided is a compound, tautomer, or stereoisomer of Formula (A) or pharmaceutically acceptable salts thereof: 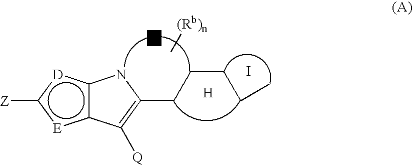
wherein:

ring H and ring I are independently an optionally substituted 6 member aryl or an optionally substituted 5 or 6 member heteroaryl having one, two, or three ring heteroatoms independently selected from the group consisting of N, NH, N-oxide, O, or S;

T is C₁ to C₅ alkylene wherein one or two —CH₂— groups are optionally replaced with —NR^{c}—, —S—, or —O— and optionally two —CH₂— groups together form a double bond provided that T does not contain an —O—O, —S—O—, or —S—S— group;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

one of D or E is C—R^{a} and the other of D or E is S; or D is CH and E is —CH═CH— such that Z, D, E and the atoms to which they are attached together form a fused 6-member ring with the remainder of the molecule:

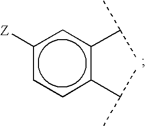

R^{a} is independently selected from the group consisting of hydrogen, alkyl, and substituted alkyl;

R^{b} is selected from the group consisting of halo, acyl, acylamino, alkyl, substituted alkyl, carboxy ester, hydroxy, and ═O;

n is 0, 1, or 2;

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In another embodiment, provided is a compound, tautomer, or stereoisomer of having Formula (I) or (II) or pharmaceutically acceptable salts thereof: 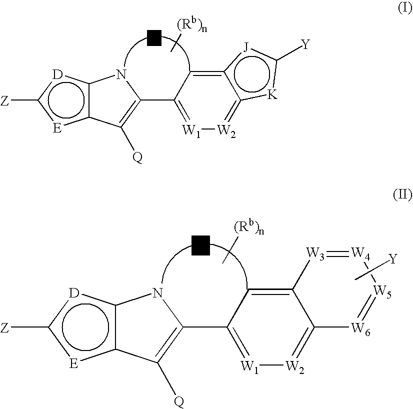
wherein:

Y is selected from the group consisting of hydrogen, alkyl, substituted alkyl, alkoxy, substituted alkoxy, amino, substituted amino, halo, hydroxy, nitro, aryl, heteroaryl, substituted aryl, and substituted heteroaryl;

J and K are independently selected from the group consisting of N, NH, CX, and N-oxide provided that J and K are not both CX;

W₁, W₂, W₃, W₄, W₅, and W₆ are independently selected from the group consisting of N,N-oxide, or CX, provided that no more than one of J, K, and W₁-W₆ is an N-oxide and provided that one of W₄ or W₅ is C—Y;

each X is independently selected from the group consisting of hydrogen, alkyl, substituted alkyl, alkoxy, substituted alkoxy, amino, substituted amino, halo, hydroxy, and nitro;

T is C₁ to C₅ alkylene wherein one or two —CH₂— groups are optionally replaced with —NR^{c}—, —S—, or —O— and optionally two —CH₂— groups together form a double bond provided that T does not contain an —O—O—, —S—O—, or —S—S— group;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

one of D or E is C—R^{a} and the other of D or E is S; or D is CH and E is —CH═CH— such that Z, D, E and the atoms to which they are attached together form a fused 6-member ring with the remainder of the molecule:

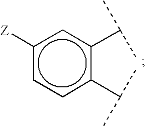

R^{a} and R^{c} are independently selected from the group consisting of hydrogen, alkyl, and substituted alkyl;

R^{b} is selected from the group consisting of halo, acyl, acylamino, alkyl, substituted alkyl, carboxy ester, hydroxy, and ═O;

n is 0, 1, or 2;

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁹ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

Other compounds of Formula (A) including a compound, tautomer, or stereoisomer of any of Formula (I)-(IV) and their subgeneric formulae or the pharmaceutically acceptable salts thereof are provided and are further described in the detailed description below.

In one embodiment provided is a pharmaceutical composition comprising a pharmaceutically acceptable carrier and a therapeutically effective amount of a compound, tautomer, or stereoisomer of Formula (A).

In one embodiment provided is a method for treating a viral infection in a mammal mediated at least in part by a virus in the Flaviviridae family of viruses, comprising administering to said mammal a composition of Formula (A). In some aspects, the viral infection is mediated by hepatitis C virus.

### DETAILED DESCRIPTION OF THE INVENTION

### Definitions

It is to be understood that the terminology used herein is for the purpose of describing particular embodiments only and is not intended to limit the scope of the present invention. In this specification and in the claims that follow, reference will be made to a number of terms that shall be defined to have the following meanings:

As used herein, “alkyl” refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, cycloalkenyl, substituted cycloalkenyl, heteroaryl, substituted heteroaryl, heterocyclic, and substituted heterocyclic.

“Alkylene” refers to divalent straight chain alkyl groups having from 1 to 5 carbons.

“Alkoxy” refers to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Acylamino” refers to the group —C(O)NR^{f}R^{g} where R^{f} and R^{g} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{f} and R^{g} are joined to form together with the nitrogen atom a heterocyclic or substituted heterocyclic ring.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

“Alkenyl” refers to alkenyl group having from 2 to 10 carbon atoms, preferably having from 2 to 6 carbon atoms, and more preferably 2 to 4 carbon atoms and having at least 1 and preferably from 1-2 sites of alkenyl unsaturation.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to a vinyl carbon atom.

“Alkynyl” refers to alkynyl group having from 2 to 10 carbon atoms, preferably having from 2 to 6 carbon atoms, and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to an acetylenic carbon atom.

“Amino” refers to the group —NH₂.

“Substituted amino” refers to the group —NR^{h}Rⁱ where R^{h} and Rⁱ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{h} and Rⁱ are joined, together with the nitrogen bound thereto to form a heterocyclic or substituted heterocyclic group provided that R^{h} and Rⁱ are both not hydrogen. When R^{h} is hydrogen and Rⁱ is alkyl, the substituted amino group is sometimes referred to herein as alkylamino. When R^{h} and Rⁱ are alkyl, the substituted amino group is sometimes referred to herein as dialkylamino.

“Aminoacyl” refers to the groups —NR^{j}C(O)alkyl, —NR^{j}C(O)substituted alkyl, —NR^{j}C(O)-cycloalkyl, —NR^{j}C(O)substituted cycloalkyl, —NR^{j}C(O)alkenyl, —NR^{j}C(O)substituted alkenyl, —NR^{j}C(O)alkynyl, —NR^{j}C(O)substituted alkynyl, —NR^{j}C(O)aryl, —NR^{j}C(O)substituted aryl, —NR^{j}C(O)heteroaryl, —NR^{j}C(O)substituted heteroaryl, —NR^{j}C(O)heterocyclic, and —NR^{j}C(O)substituted heterocyclic where R^{j} is hydrogen or alkyl.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl.

“Aralkyl” or “arylalkyl” refers to the group aryl-alkyl- and includes, for example, benzyl.

“Substituted aryl” refers to aryl groups which are substituted with from 1 to 3 substituents, and preferably 1 to 2 substituents, selected from the group consisting of hydroxy, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy ester, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Arylene” and “substituted arylene” refer to divalent aryl and substituted aryl groups as defined above. “Phenylene” is a 6-membered optionally substituted arylene group and includes, for example, 1,2-phenylene, 1,3-phenylene, and 1,4-phenylene.

“Aryloxy” refers to the group aryl-O— that includes, by way of example, phenoxy, naphthoxy, and the like.

“Substituted aryloxy” refers to substituted aryl-O— groups.

“Carboxy” refers to —C(═O)OH or salts thereof.

“Carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic. Preferred carboxy ester are —C(O)O-alkyl, —C(O)O-substituted alkyl, —C(O)O-aryl, and —C(O)O-substituted aryl.

“Cycloalkyl” refers to cyclic alkyl groups of from 3 to 10 carbon atoms having single or multiple cyclic rings optionally comprising 1 to 3 exo carbonyl or thiocarbonyl groups. Suitable cycloalkyl groups include, by way of example, adamantyl, cyclopropyl, cyclobutyl, cyclopentyl, cyclohexyl, cyclooctyl, 3-oxocyclohexyl, and the like. In multiple condensed rings, one or more of the rings may be other than cycloalkyl (e.g., aryl, heteroaryl or heterocyclic) provided that the point of attachment is to a carbon ring atom of the cycloalkyl group. In one embodiment, the cycloalkyl group does not comprise 1 to 3 exo carbonyl or thiocarbonyl groups. In another embodiment, the cycloalkyl group does comprise 1 to 3 exo carbonyl or thiocarbonyl groups. It is understood, that the term “exo” refers to the attachment of a carbonyl or thiocarbonyl to a carbon ring atom of the cycloalkyl group.

“Substituted cycloalkyl” refers to a cycloalkyl group, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic.

“Cycloalkenyl” refers to cyclic alkenyl but not aromatic groups of from 5 to 10 carbon atoms having single or multiple cyclic rings optionally comprising 1 to 3 exo carbonyl or thiocarbonyl groups. Suitable cycloalkenyl groups include, by way of example, cyclopentyl, cyclohexenyl, cyclooctenyl, 3-oxocyclohexenyl, and the like. In multiple condensed rings, one or more of the rings may be other than cycloalkenyl (e.g., aryl, heteroaryl or heterocyclic) provided that the point of attachment is to a carbon ring atom of the cycloalkyl group. In one embodiment, the cycloalkenyl group does not comprise 1 to 3 exo carbonyl or thiocarbonyl groups. In another embodiment, the cycloalkenyl group does comprise 1 to 3 exo carbonyl or thiocarbonyl groups. It is understood, that the term “exo” refers to the attachment of a carbonyl or thiocarbonyl to a carbon ring atom of the cycloalkenyl group.

“Substituted cycloalkenyl” refers to cycloalkenyl groups having from 1 to 5 substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that for hydroxyl substituents the point of attachment is not to a vinyl carbon atom.

“Cycloalkoxy” refers to —O-cycloalkyl groups.

“Substituted cycloalkoxy” refers to —O-substituted cycloalkyl groups.

The term “guanidino” refers to the group —NHC(═NH)NH₂ and the term “substituted guanidino” refers to —NR^{c}(═NR)N(R^{p})₂ where each R^{p} is independently hydrogen or alkyl.

“Halo” or “halogen” refers to fluoro, chloro, bromo and iodo and preferably is fluoro or chloro.

“Haloalkyl” refers to an alkyl group substituted with 1 to 5 halogen groups. An example of haloalkyl is CF₃.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur, within the ring. Preferably, such heteroaryl groups are aromatic groups of from 1 to 15 carbon atoms, preferably from 1 to 10 carbon atoms, and 1 to 4 heteroatoms selected from the group consisting of oxygen, nitrogen, and sulfur within the ring. Such heteroaryl groups can have a single ring (e.g., pyridyl or furyl) or multiple condensed rings (e.g., indolizinyl or benzothienyl). The sulfur atom(s) in the heteroaryl group may optionally be oxidized to sulfoxide and sulfone moieties.

“Substituted heteroaryl” refers to heteroaryl groups that are substituted with from 1 to 3 substituents selected from the same group of substituents defined for substituted aryl.

When a specific heteroaryl is defined as “substituted”, e.g., substituted qunioline, it is understood that such a heteroaryl contains the 1 to 3 substituents as recited above.

“Heteroarylene” and “substituted heteroarylene” refer to divalent heteroaryl and substituted heteroaryl groups as defined above.

“Heteroaryloxy” refers to the group —O-heteroaryl and “substituted heteroaryloxy” refers to the group —O-substituted heteroaryl.

“Heterocycle” or “heterocyclic” or “heterocyclyl” refers to a saturated or unsaturated but not aromatic group having a single ring or multiple condensed rings, from 1 to 10 carbon atoms and from 1 to 4 hetero atoms selected from the group consisting of nitrogen, sulfur or oxygen within the ring which ring may optionally comprise 1 to 3 exo carbonyl or thiocarbonyl groups. Preferably, such heterocyclic groups are saturated or unsaturated group having a single ring or multiple condensed rings, from 1 to 10 carbon atoms and from 1 to 4 hetero atoms selected from the group consisting of nitrogen, sulfur, or oxygen within the ring. The sulfur atom(s) in the heteroaryl group may optionally be oxidized to sulfoxide and sulfone moieties.

In multiple condensed rings, one or more of the rings may be other than heterocyclic (e.g., aryl, heteroaryl or cycloalkyl) provided that the point of attachment is to a heterocyclic ring atom. In one embodiment, the heterocyclic group does not comprise 1 to 3 exo carbonyl or thiocarbonyl groups. In a preferred embodiment, the heterocyclic group does comprise 1 to 3 exo carbonyl or thiocarbonyl groups. It is understood, that the term “exo” refers to the attachment of a carbonyl or thiocarbonyl to a carbon ring atom of the heterocyclic group.

“Substituted heterocyclic” refers to heterocycle groups that are substituted with from 1 to 3 of the same substituents as defined for substituted cycloalkyl. Preferred substituents for substituted heterocyclic groups include heterocyclic groups having from 1 to 5 having substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic.

When a specific heterocyclic is defined as “substituted”, e.g., substituted morpholino, it is understood that such a heterocycle contains the 1 to 3 substituents as recited above.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

The term “thiol” refers to the group —SH.

“Isosteres” are different compounds that have different molecular formulae but exhibit the same or similar properties. For example, tetrazole is an isostere of carboxylic acid —COOH because it mimics the properties of carboxylic acid even though they both have very different molecular formulae. Tetrazole is one of many possible isosteric replacements for carboxylic acid. Other carboxylic acid isosteres of the present invention include —SO₃H, —SO₂HNR^{k}, —PO₂(R^{k})₂, —CN, —PO₃(R^{k})₂, —OR^{k}, —SR^{k}, —NHCOR^{k}, —N(R^{k})₂, —CON(R^{k})₂, —CONH(O)R^{k}, —CONHNHSO₂R^{k}, —COHNSO₂R^{k}, and —CONR^{k}CN, where R^{k} is selected from hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, thioalkyl, alkylthio, substituted sulfonyl, alkyl, alkenyl or alkynyl, aryl, aralkyl, cycloalkyl, heteroaryl, heterocycle, and CO₂R^{m} where R^{m} is alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred isosteres contemplated by this invention: 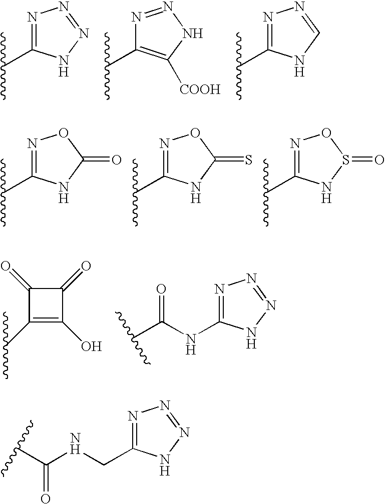
where the atoms of said ring structure may be optionally substituted at one or more positions with R^{k}. The present invention contemplates that when chemical substituents are added to a carboxylic isostere then the inventive compound retains the properties of a carboxylic isostere. The present invention contemplates that when a carboxylic isostere is optionally substituted with one or more moieties selected from R^{k}, then the substitution cannot eliminate the carboxylic acid isosteric properties of the inventive compound. The present invention contemplates that the placement of one or more R^{k} substituents upon the carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

“Carboxylic acid bioisosteres” are compounds that behave as isosteres of carboxylic acids under biological conditions.

Other carboxylic acid isosteres not specifically exemplified or described in this specification are also contemplated by the present invention

“Metabolite” refers to any derivative produced in a subject after administration of a parent compound. The metabolite may be produced from the parent compound by various biochemical transformations in the subject such as, for example, oxidation, reduction, hydrolysis, or conjugation. Metabolites include, for example, oxides and demethylated derivatives.

“Thiocarbonyl” refers to the group C(═S).

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Prodrug” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

“Treating” or “treatment” of a disease in a refers to 1) preventing the disease from occurring in a patient that is predisposed or does not yet display symptoms of the disease; 2) inhibiting the disease or arresting its development; or 3) ameliorating or causing regression of the disease. “Patient” refers to mammals and includes humans and non-human mammals.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—; the term “alkylaryloxy” refers to the group alkyl-aryl-O—; the term “arylalkyloxy” refers to the group aryl-alkyl-O—, “thioalkyl” refers to HS-alkyl-; “alkylthio” refers to alkyl-S— etc. Various substituents may also have alternate but equivalent names. For example, the term 2-oxo-ethyl and the term carbonylmethyl both refer to the —C(O)CH₂— group.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups or a hydroxy group alpha to ethenylic or acetylenic unsaturation). Such impermissible substitution patterns are well known to the skilled artisan.

Accordingly, provided is a compound, tautomer, or stereoisomer of Formula (A) or pharmaceutically acceptable salts thereof: 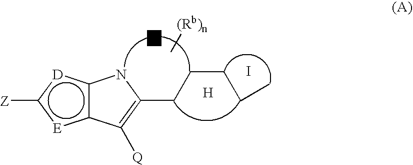
wherein:

ring H and ring I are independently an optionally substituted 6 member aryl or an optionally substituted 5 or 6 member heteroaryl having one, two, or three ring heteroatoms independently selected from the group consisting of N, NH, N-oxide, O, or S;

T is C₁ to C₅ alkylene wherein one or two —CH₂— groups are optionally replaced with —NR^{c}—, —S—, or —O— and optionally two —CH₂— groups together form a double bond provided that T does not contain an —O—O—, —S—O—, or —S—S— group;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

one of D or E is C—R^{a} and the other of D or E is S; or D is CH and E is —CH═CH— such that Z, D, E and the atoms to which they are attached together form a fused 6-member ring with the remainder of the molecule:

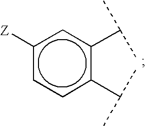

R^{a} is independently selected from the group consisting of hydrogen, alkyl, and substituted alkyl;

R^{b} is selected from the group consisting of halo, acyl, acylamino, alkyl, substituted alkyl, carboxy ester, hydroxy, and ═O;

n is 0, 1, or 2;

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁹ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In another embodiment, provided is a compound, tautomer, or stereoisomer of having Formula (I) or (II) or pharmaceutically acceptable salts thereof: 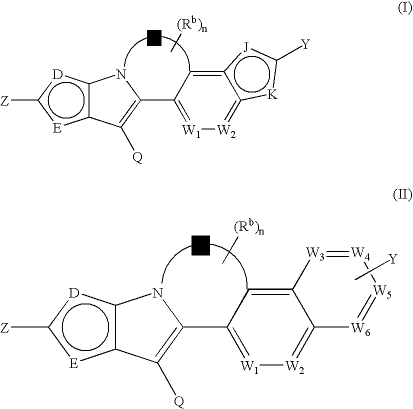
wherein:

Y is selected from the group consisting of hydrogen, alkyl, substituted alkyl, alkoxy, substituted alkoxy, amino, substituted amino, halo, hydroxy, nitro, aryl, heteroaryl, substituted aryl, and substituted heteroaryl;

J and K are independently selected from the group consisting of N, NH, CX, and N-oxide provided that J and K are not both CX;

W₁, W₂, W₃, W₄, W₅, and W₆ are independently selected from the group consisting of N,N-oxide, or CX, provided that no more than one of J, K, and W₁-W₆ is an N-oxide and provided that one of W₄ or W₅ is C—Y;

each X is independently selected from the group consisting of hydrogen, alkyl, substituted alkyl, alkoxy, substituted alkoxy, amino, substituted amino, halo, hydroxy, and nitro;

T is C₁ to C₅ alkylene wherein one or two —CH₂— groups are optionally replaced with —NR^{c}—, —S—, or —O— and optionally two —CH₂— groups together form a double bond provided that T does not contain an —O—O—, —S—O—, or —S—S— group;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

one of D or E is C—R^{a} and the other of D or E is S; or D is CH and E is —CH═CH— such that Z, D, E and the atoms to which they are attached together form a fused 6-member ring with the remainder of the molecule:

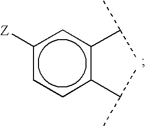

R^{a} and R^{c} are independently selected from the group consisting of hydrogen, alkyl, and substituted alkyl;

R^{b} is selected from the group consisting of halo, acyl, acylamino, alkyl, substituted alkyl, carboxy ester, hydroxy, and ═O;

n is 0, 1, or 2;

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁹ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In another embodiment T is selected from the group consisting of —CH₂CH₂CH₂—, —CH₂CH═CH—, —CH₂CH₂CH₂CH₂—, —CH₂NR^{c}CH₂—, and —CH₂CH₂NR^{c}CH₂—.

In another embodiment provided is a compound having Formula (Ia) or (IIa)

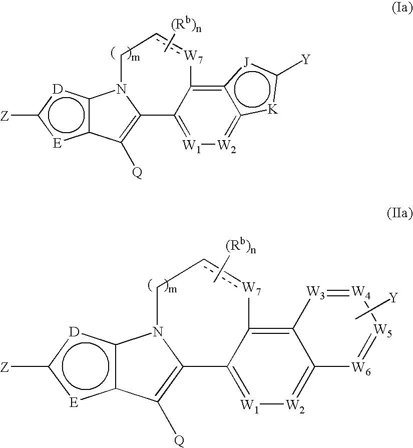

wherein W₇ is selected from the group consisting of CH, CH₂, NR^{c}, and O; m is 0, 1, or 2; the line [symbol] represents a single bond when W₇ is N or CH₂ or double bond when W₇ is CH; and

Z, D, E, Q, R^{b}, R^{c}, n, J, K, W₁, W₂, W₃, W₄, W₅, W₆, and Y are previous defined.

In another embodiment provided is a compound having Formula (Ib) or (IIb)

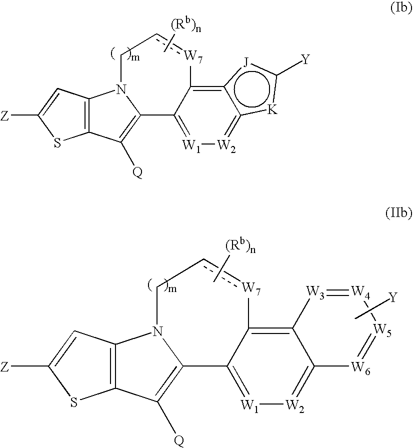

wherein Z, Q, R^{b}, n, m, J, K, W₁, W₂, W₃, W₄, W₅, W₆, W₇ and Y are previous defined.

In another embodiment provided is a compound having Formula (Ic) or (IIc)

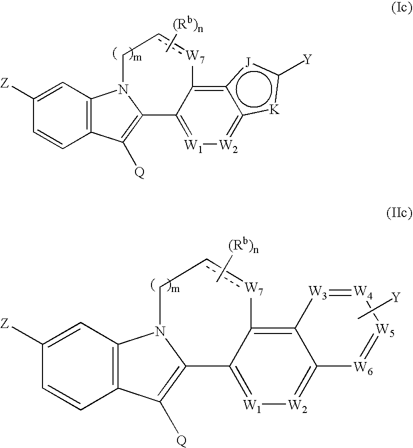

wherein Z, Q, R^{b}, n, m, J, K, W₁, W₂, W₃, W₄, W₅, W₆, W₇ and Y are previous defined.

In another embodiment provided is a compound having Formula (Id) or (IId)

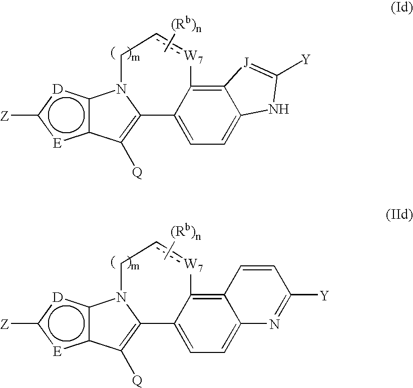

wherein J is CX or N, and Z, D, E, Q, R^{b}, n, m, W₇, Y are previous defined.

In another embodiment provided is a compound having Formula (Ie) or (IIe)

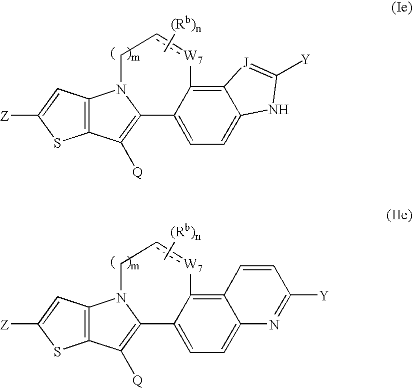

wherein J is CX or N, and Z, Q, R^{b}, n, m, W₇, Y are previous defined.

In another embodiment provided is a compound having Formula (If) or (IIf)

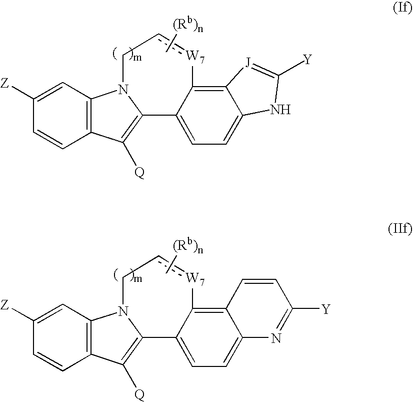

wherein J is CX or N, and Z, Q, R^{b}, n, m, W₇, Y are previous defined.

In another embodiment, provided is a compound, tautomer, or stereoisomer having Formula

(III) or (IV) or pharmaceutically acceptable salts thereof: 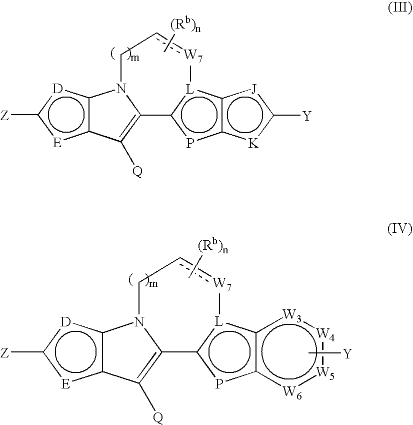
wherein:

Y is selected from the group consisting of hydrogen, alkyl, substituted alkyl, alkoxy, substituted alkoxy, amino, substituted amino, halo, hydroxy, nitro, aryl, heteroaryl, substituted aryl, and substituted heteroaryl;

J and K are independently selected from the group consisting of N, NH, CX, and N-oxide provided that J and K are not both CX;

when L is C, P is NH;

when L is N, P is N or CX and W₇ is CH or CH₂;

W₃, W₄, W₅, and W₆, are independently selected from the group consisting of N,N-oxide, or CX, provided that no more than one of J, K, and W₃-W₆ is an N-oxide and provided that one of W₄ or W₅ is C—Y;

m is 0, 1, or 2;

the line [symbol] represents a single bond when W₇ is N or CH₂ or a double bond when W₇ is CH;

W₇ is selected from the group consisting of CH, CH₂, and NR^{c};

each X is independently selected from the group consisting of hydrogen, alkyl, substituted alkyl, alkoxy, substituted alkoxy, amino, substituted amino, halo, hydroxy, and nitro;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl;

one of D or E is C—R^{a} and the other of D or E is S; or D is CH and E is —CH═CH— such that Z, D, E and the atoms to which they are attached together form a fused 6-member ring with the remainder of the molecule:

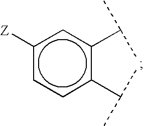

R^{a} and R^{c} are independently selected from the group consisting of hydrogen, alkyl, and substituted alkyl;

R^{b} is selected from the group consisting of halo, acyl, acylamino, alkyl, substituted alkyl, carboxy ester, hydroxy, and ═O;

n is 0, 1, or 2; and

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁹ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In another embodiment, provided is a compound, tautomer, or stereoisomer having Formula (IVa) or pharmaceutically acceptable salts thereof:

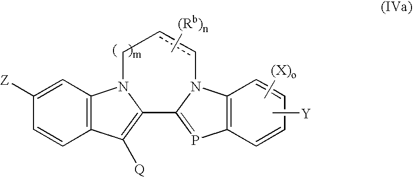

wherein Z, Q, R^{b}, n, m, X, and Y are previous defined, the line [symbol] represents a single bond or double bond, P is N or CH, and o is 0, 1, 2, or 3.

In some embodiments P is CH.

Various features relating to the embodiments above are given below. These features when referring to different substituents or variables can be combined with each other or with any other embodiments described in this application.

In some embodiments J is CH.

In some embodiments J is N.

In some embodiments E is S. In other embodiments, D is CH and E is S.

In some embodiments R^{a} is hydrogen. In other embodiments, R^{a} is substituted alkyl, substituted amino, or substituted aminoalkyl. In some aspects, R^{a} is selected from the following substituents:

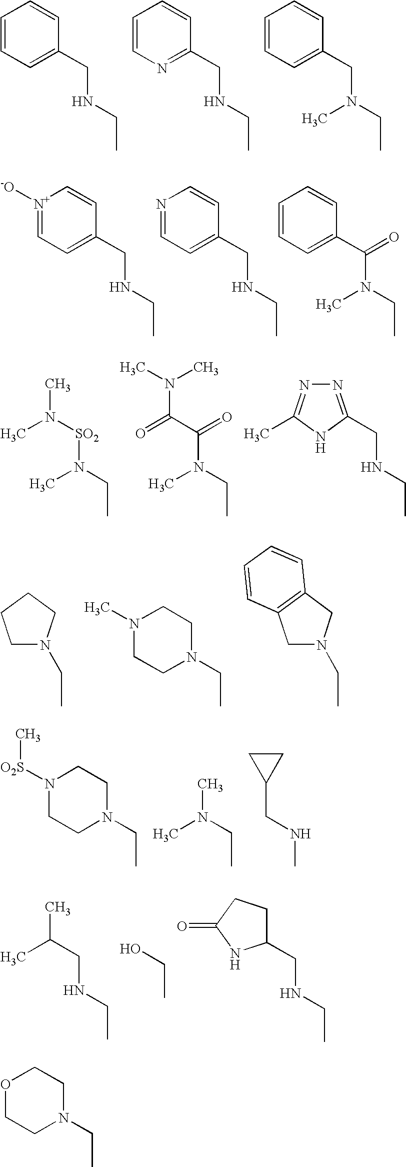

In some embodiments of Q is cycloalkyl or substituted cycloalkyl. In some embodiments Q is cycloalkyl. In another embodiment Q is cyclohexyl or substituted cyclohexyl. In another embodiment Q is 2-fluorocyclohexyl.

In some embodiments Z is carboxy or carboxy ester. In another embodiment Z is selected from —C(═O)OH, and —C(═O)OR″ where R″ is alkyl. In another embodiment Z is selected from carboxy, methyl carboxylate, and ethyl carboxylate. In yet another embodiment Z is —C(═O)OH.

In another embodiment Z is a carboxylic acid isostere. In another embodiment the carboxylic acid isostere is a carboxylic acid bioisostere. In another embodiment the carboxylic acid isostere is selected from 1H-tetrazol-5-yl and 5-oxo-4,5-dihydro-1,2,4-oxadiazol-3-yl.

In another embodiment Z is —C(═O)NR⁸R⁹ where R⁸ is hydrogen and R⁹ is selected from the group consisting of alkyl, substituted alkyl, aryl, substituted aryl, heteroaryl, substituted heteroaryl, heterocyclic and substituted heterocyclic. In another embodiment where Z is —C(═O)NR⁸R⁹ and R⁸ is hydrogen, R⁹ is substituted alkyl. In another embodiment where Z is —C(═O)NR⁸R⁹ and R⁸ is hydrogen, and R⁹ is substituted alkyl, the substituted alkyl comprises 1 to 2 substituents selected from the group consisting of sulfonic acid (SO₃H), carboxy, carboxy ester, amino, substituted amino, aryl, substituted aryl, heteroaryl and substituted heteroaryl. In another embodiment where Z is —C(═O)NR⁸R⁹ and R⁸ is hydrogen, and R⁹ is substituted alkyl, the substituted alkyl group is selected from the group consisting of 3,4-dimethoxybenzyl, 3,4-dihydroxybenzyl, 3-methoxy-4-hydroxybenzyl, 4-aminosulfonylbenzyl, 4-methylsulfonylbenzyl, (1-methyl-piperidin-3-yl)methyl, (1-methyl-pyrrolidin-3-yl)methyl, fur-2-ylmethyl, 6-methylpyridin-2-ylmethyl, 2-(1-methyl-pyrrolidin-3-yl)ethyl, 1-phenylethyl, 1-(3-methoxyphenyl)-ethyl, 1-(4-methoxyphenyl)-ethyl, N′,N′-dimethylaminoethyl, and 2-(1H-pyrazol-1-yl)ethyl.

In another embodiment Z is selected from N-methyl carboxamide, N,N-dimethylcarboxamido, N-isopropyl-carboxamido, N-allyl-carboxamido, and 5-hydroxy-tryptophan-carbonyl.

In another embodiment Z is —C(═O)NR⁸R⁹ wherein R⁹ is aryl or substituted aryl. In another embodiment where Z is —C(═O)NR⁸R⁹, R⁹ is substituted aryl. In another embodiment where Z is —C(═O)NR⁸R⁹, R⁹ is selected from the group consisting of 7-hydroxynaphth-1-yl, 6-hydroxynaphth-1-yl, 5-hydroxynaphth-1-yl, 6-carboxynaphth-2-yl, (4-HOOCCH₂-)phenyl, (3,4-dicarboxy)phenyl, 3-carboxyphenyl, 3-carboxy-4-hydroxyphenyl and 2-biphenyl.

In another embodiment Z is —C(═O)NR⁸R⁹ where R⁹ is heteroaryl or substituted heteroaryl. In another embodiment where Z is —C(═O)NR⁸R⁹, R⁹ is substituted heteroaryl. In another embodiment where Z is —C(═O)NR⁸R⁹ and R⁹ is substituted heteroaryl, the substituted heteroaryl is selected from the group consisting of 4-methyl-2-oxo-2H-chromen-7-yl, 1-phenyl-4-carboxy-1H-pyrazol-5-yl, 5-carboxypyrid-2-yl, 2-carboxypyrazin-3-yl, and 3-carboxythien-2-yl.

In another embodiment Z is —C(═O)NR⁸R⁹ where R⁹ is heterocyclic. In another embodiment where Z is —C(═O)NR⁸R⁹ and R⁹ is heterocyclic, the heterocyclic group is N-morpholino, tetrahydrofuranyl, and 1,1-dioxidotetrahydrothienyl.

In another embodiment Z is —C(═O)NR⁸R⁹ where R⁸ and R⁹, together with the nitrogen atom pendent thereto, form a heterocyclic or substituted heterocyclic ring. In another embodiment where Z is —C(═O)NR⁸R⁹ and R⁸ and R⁹, together with the nitrogen atom pendent thereto form a ring, the heterocyclic and substituted heterocyclic rings comprise 4 to 8 membered rings containing 1 to 3 heteroatoms. In another embodiment where Z is —C(═O)NR⁸R⁹ and R⁸ and R⁹, together with the nitrogen atom pendent thereto form an optionally substituted heterocyclic ring, the 1 to 3 heteroatoms comprises 1 to 2 nitrogen atoms. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of piperidine, substituted piperidine, piperazine, substituted piperazine, morpholino, substituted morpholino, thiomorpholino and substituted thiomorpholino wherein the sulfur atom of the thiomorpholino or substituted thiomorpholino ring is optionally oxidized to provide for sulfoxide and sulfone moieties. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of 4-hydroxypiperidin-1-yl, 1,2,3,4-tetrahydro-3-carboxy-isoquinolin-2-yl, 4-methylpiperizin-1-yl, morpholin-4-yl, thiomorpholin-4-yl, 4-methyl-piperazin-1-yl, and 2-oxo-piperazinyl.

In another embodiment, Z is —C(X)N(R³)CR²R^{2′}C(═O)R¹.

In another embodiment, Z is —C(O)NHCHR²C(═O)R¹.

In another embodiment when Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, substituted cycloalkyl, aryl, substituted aryl, heteroaryl and substituted heteroaryl. In another embodiment where Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, and substituted cycloalkyl. In another embodiment where Z is —C(X)N(R³)CR²R^{2′}C(═O)R¹ or —C(O)NHCHR²C(═O)R¹, R² is selected from the group consisting of hydrogen, methyl, 1-methylprop-1-yl, sec-butyl, hydroxymethyl, 1-hydroxyeth-1-yl, 4-amino-n-butyl, 2-carboxyeth-1-yl, carboxymethyl, benzyl, (1H-imidazol-4-yl)methyl, (4-phenyl)benzyl, (4-phenylcarbonyl)benzyl, cyclohexylmethyl, cyclohexyl, 2-methylthioeth-1-yl, iso-propyl, carbamoylmethyl, 2-carbamoyleth-1-yl, (4-hydroxy)benzyl, and 3-guanidino-n-propyl.

In another embodiment when Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino, and R² and R³, together with the carbon atom and nitrogen atom bound thereto respectively, are joined to form a heterocyclic or substituted heterocyclic group. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino and R² and R³, together with the carbon atom and nitrogen atom bound thereto respectively, are joined to form a heterocyclic or substituted heterocyclic group, the heterocyclic and substituted heterocyclic groups are selected from the group consisting of pyrrolidinyl, 2-carboxy-pyrrolidinyl, 2-carboxy-4-hydroxypyrrolidinyl, and 3-carboxy-1,2,3,4-tetrahydroisoquinolin-3-yl.

In another embodiment, Z is selected from 1-carboxamidocyclopent-1-ylaminocarbonyl, 1-carboxamido-1-methyl-eth-1-ylaminocarbonyl, 5-carboxy-1,3-dioxan-5-ylaminocarbonyl, 1-(N-methylcarboxamido)-1-(methyl)-eth-1-ylaminocarbonyl, 1-(N,N-dimethylcarboxamido)-1-(methyl)-eth-1-ylaminocarbonyl, 1-carboxy-1-methyl-eth-1-ylaminocarbonyl, 1-(N-methylcarboxamido)-cyclobutanaminocarbonyl, 1-carboxamido-cyclobutanaminocarbonyl, 1-(N,N-dimethylcarboxamido)-cyclobutanaminocarbonyl, 1-(N-methylcarboxamido)-cyclopentanaminocarbonyl, 1-(N,N-dimethylcarboxamido)-cyclopentanaminocarbonyl, 1-(carboxamido)-cyclopentanaminocarbonyl, 3-[N-(4-(2-aminothiazol-4-yl)phenyl)aminocarbonyl]-piperidin-3-ylaminocarbonyl, 3-carboxamido-pyrrolidin-3-ylaminocarbonyl, [1-(4-(acrylic acid)-phenyl)aminocarbonyl)-cyclobutan-1-yl]aminocarbonyl, and [1-methyl-1-(4-(acrylic acid)-phenyl)aminocarbonyl)-eth-1-yl]aminocarbonyl.

In another embodiment, Z is —C(O)NR²¹S(O)₂R⁴. In another embodiment where Z is —C(O)NR²¹S(O)₂R⁴, R⁴ is selected from the group consisting of alkyl, substituted alkyl, aryl, substituted aryl, heteroaryl and substituted heteroaryl. In another embodiment where Z is —C(O)NR²¹S(O)₂R⁴, R⁴ is methyl, ethyl, isopropyl, propyl, trifluoromethyl, 2,2,2-trifluoroethyl, phenyl, benzyl, phenethyl, 4-bromophenyl, 4-nitrophenyl or 4-methylphenyl, 4-methoxyphenyl, 2-aminoethyl, 2-(dimethylamino)ethyl, 2-N-benzyloxyaminoethyl, pyridinyl, thienyl, 2-chlorothien-5-yl, 2-methoxycarbonylphenyl, naphthyl, 3-chlorophenyl, 2-bromophenyl, 2-chlorophenyl, 4-trifluoromethoxyphenyl, 2,5-difluorophenyl, 4-fluorophenyl, 2-methylphenyl, 6-ethoxybenzo[d]thiazo-2-yl, 4-chlorophenyl, 3-methyl-5-fluorobenzo[b]thiophen-1-yl, 4-acetylaminophenyl, quinolin-8-yl, 4-t-butylphenyl, cyclopropyl, 2,5-dimethoxyphenyl, 2,5-dichloro-4-bromo-thien-3-yl, 2,5-dichloro-thien-3-yl, 2,6-dichlorophenyl, 1,3-dimethyl-5-chloro-1H-pyrazol-4-yl, 3,5-dimethylisoxazol-4-yl, benzo[c][1,2,5]thiadiazol-4-yl, 2,6-difluorophenyl, 6-chloro-imidazo[2,1-b]thiazol-5-yl, 2-(methylsulfonyl)phenyl, isoquinolin-8-yl, 2-methoxy-4-methylphenyl, 1,3,5-trimethyl-1H-pyrazol-4-yl, 1-phenyl-5-methyl-1H-pyrazol-4-yl, 2,4,6-trimethylphenyl, and 2-carbamoyl-eth-1-yl.

In another embodiment, Z is selected from hydrogen, halo, alkyl, alkoxy, amino, substituted amino, and cyano.

In another embodiment, Z is —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group.

In another embodiment, Z is selected from 1-(6-(3-carboxyprop-2-en-1-yl)-1H-benzo[d]imidazol-2-yl)cyclobutanaminocarbonyl, 3-(6-(3-carboxyprop-2-en-1-yl)-1H-benzo[d]imidazol-2-yl)-1-methylpyrrolidin-3-aminocarbonyl, 1-(1-methyl-6-(3-carboxyprop-2-en-1-yl)-1H-benzo[d]imidazol-2-yl)cyclobutanaminocarbonyl, 1-(benzofuran-2-yl)-5-carboxy-cyclobutanaminocarbonyl, 1-(2-methylthiazol-4-yl)-cyclobutanaminocarbonyl, 1-(2-acetylamino-thiazol-4-yl)-cyclobutanamino, 1-(2-methylamino-thiazol-4-yl)-cyclobutanaminocarbonyl, 1-(2-ethylthiazol-4-yl)-cyclobutanaminocarbonyl, and 1-(cyano)-cyclobutanaminocarbonyl.

In still other embodiments Z is carboxy, carboxy ester, carboxylic acid isostere, —C(O)NR⁸R⁹, or —C(O)NHS(O)₂R⁴, wherein R⁸ and R⁹ are as defined above and R⁴ is alkyl or aryl. In other embodiments Z is carboxy, methyl carboxylate, ethyl carboxylate, 6-(D-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl. In still other embodiments Z is carboxy. In yet other embodiments Z is —C(═O)OH.

In another embodiment, Z is selected from the group consisting of

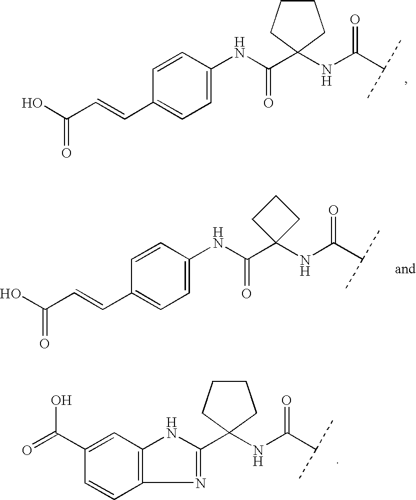

In some embodiments, Y is substituted aryl or substituted heteroaryl.

In some embodiments, Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized. In some embodiments Y is substituted 5-membered heteroaryl ring optionally fused to a phenyl ring and having one, two, or three heteroatoms independently selected from the group consisting of N, O, or S wherein the heteroatoms N or S are optionally oxidized.

In another embodiment —Y is —Ar¹-(G¹)_{q} where Ar¹ is selected from arylene and heteroarylene, G¹ is selected from halo, hydroxy, nitro, cyano, alkyl, substituted alkyl, alkoxy, substituted alkoxy, acyl, acylamino, aminoacyl, amino, substituted amino, carboxy and carboxy ester; and q is an integer from 1 to 3. In another embodiment where —Y is —Ar¹-(G¹)_{q, Ar}¹ is selected from phenyl, thiazolyl, furanyl, thienyl, pyridinyl, pyrazinyl, oxazolyl, isoxazolyl, pyrrolyl, imidazolyl, and pyrrolidinyl. In another embodiment where —Y is —Ar¹-(G¹)_{q}, G¹ is selected from bromo, chloro, methyl, hydroxy, methoxy, ethoxy, acetyl, acetamido, carboxy, and amino. In another embodiment Y is selected from 2,4-dimethylthiazol-5-yl, 3-bromo-4-aminophenyl, 3-amido-4-hydroxy-phenyl, 2-hydroxy-6-methoxy-phenyl, 4-(acetylamino)-phenyl, 2,4-dihydroxyphenyl, 2,4-dimethoxy-6-hydroxyphenyl, and 7-hydroxybenzofuranyl.

In another embodiment Y is —Ar¹—Ar²— where the —Ar¹—Ar²— group is selected from the group consisting of -aryl-aryl, -aryl-substituted aryl, -substituted aryl-aryl, -substituted aryl-substituted aryl, -aryl-heteroaryl, -aryl-substituted heteroaryl, -substituted aryl-heteroaryl, -substituted aryl-substituted heteroaryl, heteroaryl-aryl, heteroaryl-substituted aryl, substituted heteroaryl-aryl, substituted heteroaryl-substituted aryl, -aryl-cycloalkyl, -aryl-substituted cycloalkyl, -substituted aryl-cycloalkyl, -substituted aryl-substituted cycloalkyl, -aryl-heterocyclic, aryl-substituted heterocyclic, substituted aryl-heterocyclic, and substituted aryl-substituted heterocyclic.

In another embodiment where Y is —Ar¹—Ar—, the —Ar¹—Ar²— group is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphenyl, 4-methoxybiphenyl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

In another embodiment where Y is —Ar¹—Ar—, the —Ar¹—Ar² group is selected from the group consisting of 4-(1H-imidazol-1-yl)phenyl, 2-furan-2-yl-5-methoxyphenyl, 5-methoxy-2-thiophen-2-ylphenyl, 2-(2,4-dimethoxypyrimidin-5-yl)-4-methoxyphenyl, 2-(pyrid-4-yl)phenyl, 3-amino-5-phenylthiophen-2-yl, 5-(4-chlorophenyl)-2-methylfuran-2-yl, 3-(4-chlorophenyl)-5-methylisoxazol-4-yl, 2-(4-chlorophenyl)-4-methylthiazol-5-yl, 3-(3,4-dichloro-phenyl)isoxazol-5-yl, 3,5-dimethyl-1-phenyl-1H-pyrazol-4-yl, 5-methyl-2-phenylthiophen-3-yl, and 1-phenyl-1H-pyrazol-4-yl.

In another embodiment where Y is —Ar¹—Ar—, the —Ar¹—Ar²— group is selected from the group consisting of 2-cyclohexyl-N,N-dimethylamino-carbonylmethyl-5-methoxyphenyl, and 4-morpholinophenyl.

In still other embodiments, Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl. In some aspects, Y is substituted with one to three substitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester. In still other aspects, Y is 2,4-dimethylthiazol-5-yl.

In some embodiments, Y is selected from

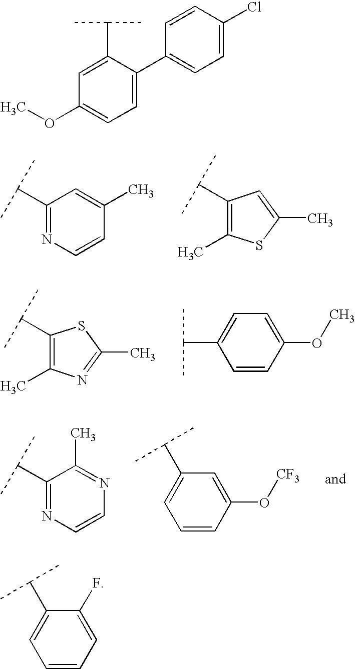

In some embodiments, Y is selected from the corresponding Y groups in Table 1.

In some embodiments, n is 1 and R^{b} is oxo.

In some embodiments, n is 2 and both R^{b} are hydroxy.

In some embodiments, R^{b} is —C(O)NR¹²R¹³ wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms. In some aspects, R¹² and R¹³ together form a morpholino ring.

In some embodiments, T is —CH₂CH═CH—.

In some embodiments, T is —CH₂CH₂CH₂—.

In some embodiments, T is —CH₂NR^{c}CH₂—.

In some embodiments, T is —CH₂CH₂NR^{c}CH₂—.

In some embodiments, T is —CH₂CH₂CH₂CH₂—.

In some embodiments, m is 0.

In some embodiments, m is 1.

In some embodiments, m is 2.

In some embodiments, W₇ is O. In some aspects, m is 1.

In some embodiments, W₇ is CH. In some aspects, m is 0. In other aspects, m is 1.

In some embodiments, W₇ is NR^{c}. In some aspects, m is 1. In other aspects, m is 2.

In some aspects, R^{c} is hydrogen. In other aspects R^{c} is alkyl substituted with heterocyclyl or substituted heterocyclyl. In still other aspects R^{c} is —C(O)O(alkyl).

In some aspects, R^{c} is selected from

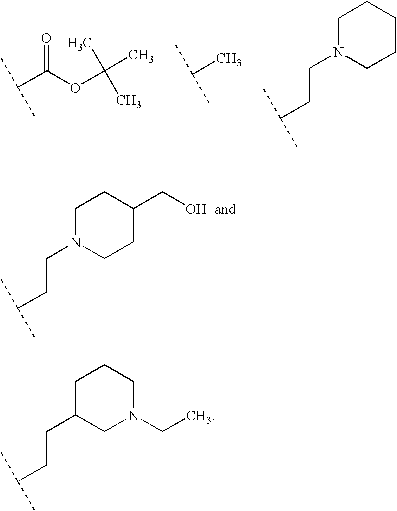

In another aspect, R^{c} is CᵥH₂ᵥ—C(O)—NR¹²R¹³ where v is 1, 2 or 3; R¹² and R¹³ are selected from hydrogen, alkyl, substituted alkyl, alkenyl, substituted alkenyl, alkynyl, substituted alkynyl alkoxy, substituted alkoxy and —(CH₂)₀₋₃R¹⁶; and R¹⁶ is aryl, heteroaryl, heterocyclic, —NR¹⁷R¹⁸; and R¹⁷ and R¹⁸ are independently selected from hydrogen, and alkyl, or alternatively, R¹⁷ and R¹⁸ together with the nitrogen atom to which they are attached join to form a heterocyclic ring with 4 to 7 ring atoms; or, alternatively, R¹² and R¹³ and the nitrogen atom to which they are attached form a heterocyclic or substituted heterocyclic ring; provided that both R¹² and R¹³ are not alkoxy and/or substituted alkoxy. In another embodiment v is 1. In another embodiment where R^{c} is CᵥH₂ᵥ—C(O)—NR¹²R¹³, the NR¹²R¹³ group is selected from N,N-dimethylamino-carbonylmethyl, [N-(4-hydroxy-1,1-dioxidotetrahydro-3-thienyl)amino]-carbonylmethyl, (cyclopropylmethylamino)-carbonylmethyl, (prop-2-yn-1-ylamino)-carbonylmethyl, (2-(morpholino)eth-1-ylamino)-carbonylmethyl, (phenylsulfonylamino)-carbonylmethyl, [N-benzylamino]-carbonylmethyl, (N-(4-methylsulfonyl-benzyl)amino)-carbonylmethyl, (tryptophanyl)-carbonylmethyl, (tyrosine)-carbonylmethyl, (N-(1-carboxyprop-1-ylamino)-carbonylmethyl, (N-(2-carboxyeth-1-yl)-amino)-carbonylmethyl, (N-(4-carboxybenzyl)-amino)-carbonylmethyl, N-[3-(N′-(4-(acrylic acid)-phenyl)carboxamido) pyrrolidin-3-yl]amino-carbonylmethyl, N-[4-(N′-(4-(acrylic acid)-phenyl)carboxamido)piperidin-4-yl]amino-carbonylmethyl, [2-(N,N-dimethylamino)eth-1-ylamino]-carbonylmethyl, [(1-(5-methyl-4H-1,2,4-triazol-3-yl)ethyl)amino]-carbonylmethyl, (1-methyl-1-[N-(1-methyl-2-carboxy-1H-indol-5-yl)aminocarbonyl]eth-1-ylamino-carbonylmethyl, [N-(1-methylpyrrolidin-3-yl-ethyl)-amino]-carbonylmethyl, (1-methyl-1-[N-(4-(acrylic acid)phenyl)aminocarbonyl]eth-1-ylamino-carbonylmethyl, (1-methyl-1-[N-(4-(2-carboxy-furan-5-yl)phenyl)aminocarbonyl]eth-1-ylamino-carbonylmethyl, (1-methyl-1-[N-(4-(4-carboxy-thiazol-2-yl)phenyl)aminocarbonyl]eth-1-ylamino-carbonylmethyl, (2-(4-methylpiperazin-1-yl)eth-1-ylamino)-carbonylmethyl, [(1-methylpyrrolidin-3-yl)methylamino]-carbonylmethyl, [N-(1-methylpiperidin-3-yl-methyl)-amino]-carbonylmethyl, (1-piperidin-1-ylcyclopentyl)methylamino]-carbonylmethyl, (1-(acetyl)-pyrrolidin-2-ylmethyl)amino)-carbonylmethyl, [(2-(N,N-dimethylamino)-carbonyl)methylamino]-carbonylmethyl, [N-(1,1-dioxidotetrahydro-3-thienyl)methylamino]-carbonylmethyl, (N-methyl-N-cyclohexyl-amino)-carbonylmethyl, (N-methyl-N-carboxymethyl-amino)-carbonylmethyl, [N-methyl-N-benzyl-amino]-carbonylmethyl, (N-methyl-N-(N′,N′-dimethylaminoacetyl)-amino)-carbonylmethyl, [N-methyl-N-phenyl-amino]-carbonylmethyl, (N-methyl-N-isopropyl-amino)-carbonylmethyl, (N-methyl-N-(N′-methylpiperidin-4-yl)amino)-carbonylmethyl, [N-methyl-N-(1-methylpiperidin-4-yl)amino]-carbonylmethyl, [N-methyl-N-(1-methylpiperidin-4-yl-methyl)-amino]-carbonylmethyl, [N-methyl-N-(1-methylpiperidin-3-yl-methyl)-amino]-carbonylmethyl, [N-methyl-N-(1-methylpyrazin-2-yl-methyl)-amino]-carbonylmethyl, [N-methyl-N-(5-methyl-1H-imidazol-2-ylmethyl)-amino]-carbonylmethyl, (N-methyl-N-[2-(hydroxy)eth-1-yl]amino)-carbonylmethyl, (N-methyl-N-[2-(N′,N′dimethylamino)eth-1-yl]amino)-carbonylmethy, N-methyl-N-[2-(N′,N′-diethylamino)eth-1-yl]amino)-carbonylmethyl, (N-methyl-N-[2-(pyridin-2-yl)eth-1-yl]amino)-carbonylmethyl, (N-methyl-N-[2-(pyridin-4-yl)eth-1-yl]amino)-carbonylmethyl, [N-methyl-N-(1-(1,3-thiazol-2-yl)ethyl)-amino]-carbonylmethyl, (N-methyl-N-[3-(N′,N′-dimethylamino)prop-1-yl]amino)-carbonylmethyl, (N-methyl-N-(1-carboxy-2-methylprop-1-yl)-amino)-carbonylmethyl, (N-ethyl-N-propyl-amino)-carbonylmethyl, (N-ethyl-N-[2-(methoxy)eth-1-yl]amino)-carbonylmethyl, (N-ethyl-N-[2-(N′,N′-diethylamino)eth-1-yl]amino)-carbonylmethyl, [7-methyl-2,7-diazaspiro[4.4]non-2-yl]-carbonylmethyl, (5-methyl-2,5-diazabicyclo[2.2.1]heptyl-2-yl)-carbonylmethyl, (4-methyl-1,4-diazepan-1-yl)-carbonylmethyl, (piperidinyl)-carbonylmethyl, (4-carboxy-piperidinyl)-carbonylmethyl, (3-carboxypiperidinyl)-carbonylmethyl, (4-hydroxypiperidinyl)-carbonylmethyl, (4-(2-hydroxyeth-1-yl)piperidin-1-yl)-carbonylmethyl, [4-(N,N-dimethylamino)-piperidin-1-yl]-carbonylmethyl, (3-(N,N-dimethylamino)-methylpiperidin-1-yl)-carbonylmethyl, (2-(2-(N,N-dimethylamino)-eth-1-yl)piperidin-1-yl)-carbonylmethyl, [4-(4-methyl-4H-1,2,4-triazol-3-yl)piperidin-1-yl]-carbonylmethyl, (4-pyrrolidinyl-piperidinyl)-carbonylmethyl, (3-pyrrolidinyl-piperidinyl)-carbonylmethyl, [4-(N,N-diethylamino)-piperidin-1-yl]-carbonylmethyl, (4-(azetidin-1-yl)-piperidin-1-yl)-carbonylmethyl, (4-(piperidin-1-yl)-piperidin-1-yl)-carbonylmethyl, (hexahydropyrrolo[1,2-a]pyrazin-2(1H)-yl)-carbonylmethyl, [(2-(N,N-dimethylamino)-methyl)morpholino]-carbonylmethyl, (3,5-dimethylmorpholino)-carbonylmethyl, (thiomorpholino)-carbonylmethyl, morpholino-carbonylmethyl, (pyrrolidinyl)-carbonylmethyl, (2-carboxy-pyrrolidin-1-yl)-carbonylmethyl, (2-(carboxy)-4-hydroxy-pyrrolidin-1-yl)-carbonylmethyl, (2-carboxamide-pyrrolidin-1-yl)-carbonylmethyl, (2-(N,N-dimethylaminocarbonyl)-pyrrolidin-1-yl)-carbonylmethyl, (3-(N′,N′-dimethylamino)-pyrrolidin-1-yl)-carbonylmethyl, (3-(N′,N′-diethylamino)-pyrrolidin-1-yl)-carbonylmethyl, (3-(pyridin-3-yl)-pyrrolidin-1-yl)-carbonylmethyl, (2-pyridin-4-ylpyrrolidin-1-yl)-carbonylmethyl, piperazin-1-yl-carbonylmethyl, (4-methylpiperazinyl)-carbonylmethyl, (4-(carboxymethyl)-piperazin-1-yl)-carbonylmethyl, (4-(2-hydroxyeth-1-yl)piperazin-1-yl)-carbonylmethyl, (4-(isopropyl)piperazin-1-yl)-carbonylmethyl, (4-(2-methoxyeth-1-yl)piperazin-1-yl)-carbonylmethyl, (4-(ethyl)piperazin-1-yl)-carbonylmethyl, (4-(N′,N′-dimethylaminoacetyl)-piperazin-1-yl)-carbonylmethyl, and (4-(6-methoxypyridin-2-yl)piperazin-1-yl)-carbonylmethyl.

In another embodiment, R^{c} is selected from morpholinocarbonylmethyl, N,N-dimethylaminocarbonylmethyl, (4-pyrrolidinyl-piperidin-1-yl)carbonylmethyl, piperazinylcarbonylmethyl. In some aspects, R^{c} is an oxide of morpholinocarbonylmethyl, N,N-dimethylaminocarbonylmethyl, (4-pyrrolidinyl-piperidin-1-yl)carbonylmethyl, piperazinylcarbonylmethyl.

In another embodiment, R^{e} is selected from [(N,N-dimethylamino)prop-2-en-1-yl]-carbonylmethyl, (N,N-dimethylpiperidin-4-aminium trifluoroacetate)acetyl, 2-(N,N-dimethylpiperidin-4-aminium trifluoroacetate)morpholino acetyl, (2-(diisopropyl)eth-1-yl)-carbonylmethyl, (pyridin-4-ylcarbonylhydrazino)-carbonylmethyl, (N-(4-carboxybenzyl)-amino)carbonylhydrazino)-carbonylmethyl, (acetylhydrazino)-carbonylmethyl, ((N′,N′-dimethylaminomethyl-carbonyl)hydrazino)-carbonylmethyl.

In still other embodiments, R^{c} is substituted alkyl, wherein said substituted alkyl is selected from the group consisting of aminoalkyl, substituted aminoalkyl, arylalkyl, substituted arylalkyl, heteroarylalkyl, substituted heteroarylalkyl, heterocyclylalkyl, substituted heterocyclylalkyl, —CH₂COOH, and —CH₂CONR¹²R¹³, wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

In other embodiments, R^{c} is —CH₂CONR¹²R¹³ and at least one of R¹² or R¹³ is alkyl, substituted alkyl, or heteroaryl. In some aspects at least one of R¹² or R¹³ is methyl, carboxymethyl, 2-hydroxyethyl, 2-morpholin-4-ylethyl, or tetrazoyl-5-yl. In other aspects R is 1-methyl-piperidin-4-yl, 1-methyl-piperidin-3-ylmethyl, and thiazol-2-yl carbamoyl methyl.

In yet other embodiments, R^{c} is —CH₂CONR¹²R¹³ and R¹² and R¹³ and the nitrogen atom to which they are attached form a substituted or unsubstituted heterocyclic ring. In some aspects R¹² and R¹³ and the nitrogen atom to which they are attached form a substituted or unsubstituted morpholino, substituted or unsubstituted piperidinyl, or a substituted or unsubstituted pyrrolidinyl ring. In other aspects the substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

In some embodiments, R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-benzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido) pyrrolidin-3-yl]amino, N-[4-(N′-(4-(acrylic acid)-phenyl)carboxamido)piperidin-4-yl]amino, 2-(N,N-dimethylamino)eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, (1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N-(N′,N′-dimethylaminoacetyl)-amino, N-methyl-N-phenyl-amino, N-methyl-N-isopropyl-amino, N-methyl-N-(N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino)eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino)-piperidin-1-yl, 3-(N,N-dimethylamino)-methylpiperidin-1-yl, 2-(2-(N,N-dimethylamino)-eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl)-pyrrolidin-1-yl, 3-(N′,N′-dimethylamino)-pyrrolidin-1-yl, 3-(N′,N′-diethylamino)-pyrrolidin-1-yl, 3-(pyridin-3-yl)-pyrrolidin-1-yl, 2-pyridin-4-ylpyrrolidin-1-yl, piperazin-1-yl, 4-methylpiperazinyl, 4-(carboxymethyl)-piperazin-1-yl, 4-(2-hydroxyeth-1-yl)piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, 4-(6-methoxypyridin-2-yl)piperazin-1-yl, and 2-dimethylaminomethylmorpholin-4-yl.

Preferred compounds of this invention or the pharmaceutically acceptable salts, partial salts, or tautomers thereof include those set forth in Table I below.

TABLE I

| | |
|---|---|
| 1 | 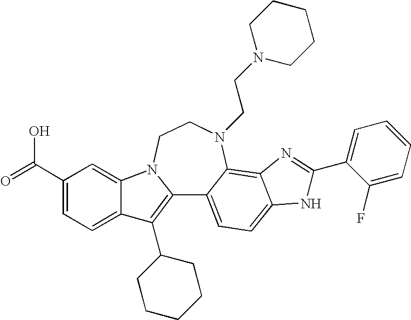 |
| 2 | 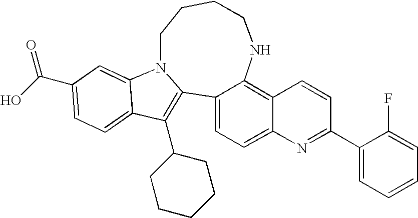 |
| 3 | 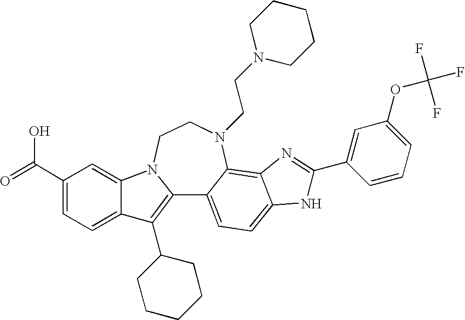 |
| 4 | 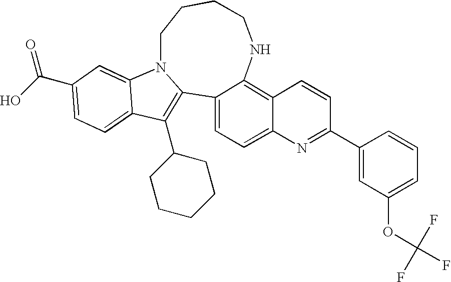 |
| 5 | 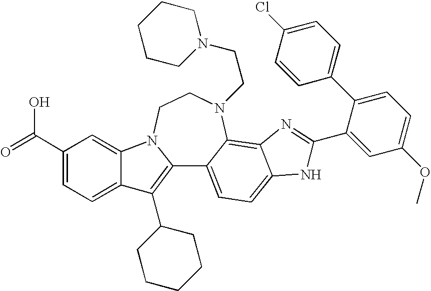 |
| 6 | 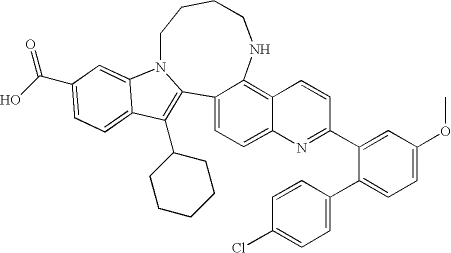 |
| 7 | 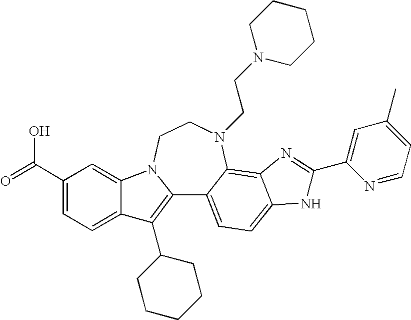 |
| 8 | 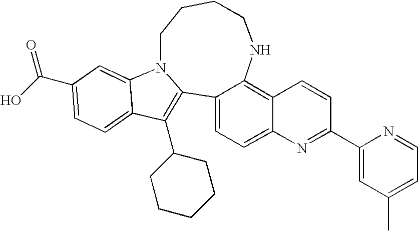 |
| 9 | 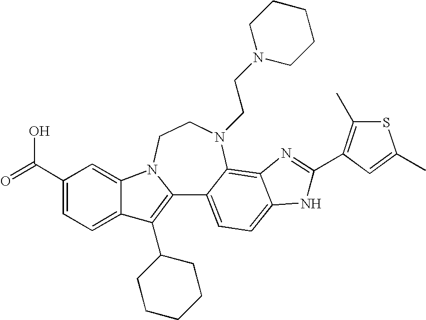 |
| 10 | 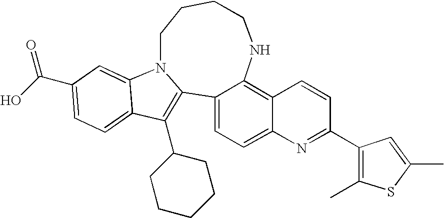 |
| 11 | 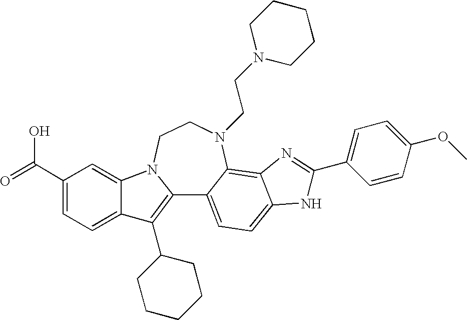 |
| 12 | 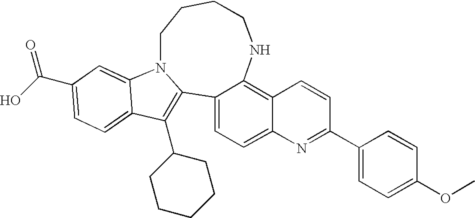 |
| 13 | 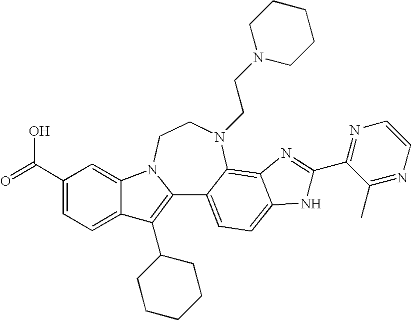 |
| 14 | 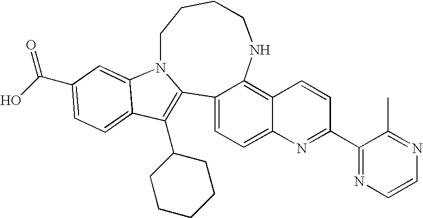 |
| 15 | 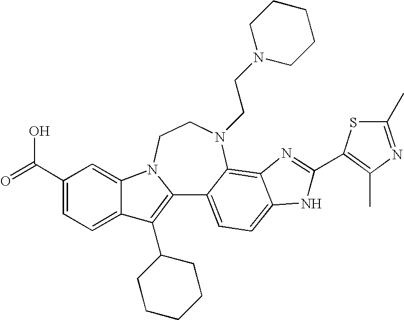 |
| 16 | 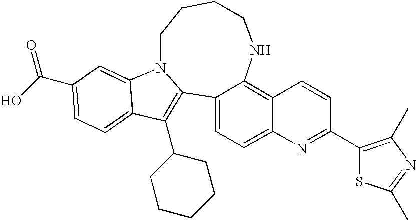 |
| 17 | 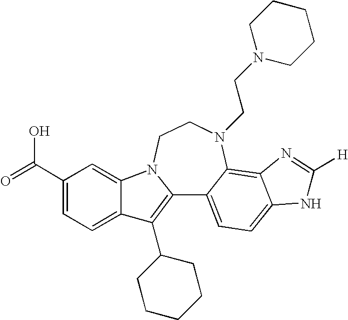 |
| 18 | 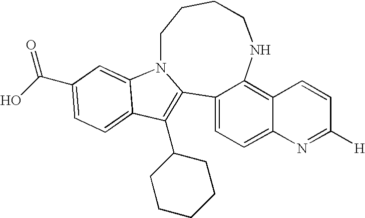 |
| 19 | 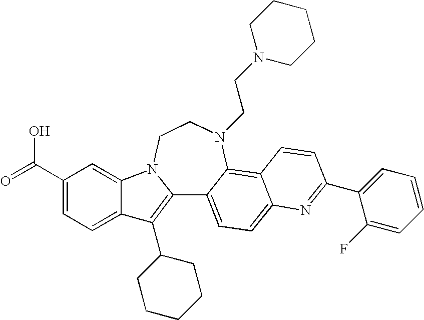 |
| 20 | 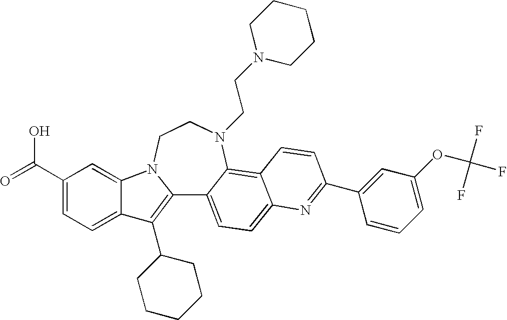 |
| 21 | 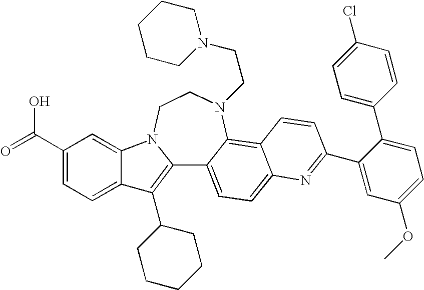 |
| 22 | 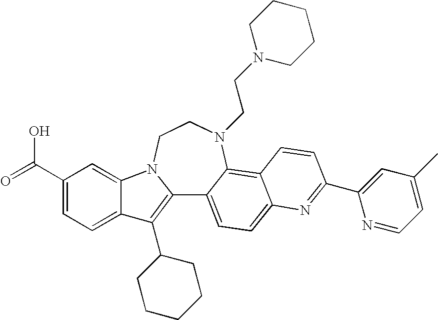 |
| 23 | 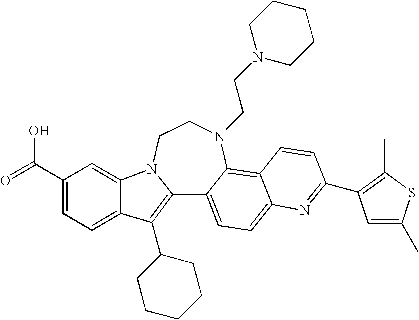 |
| 24 | 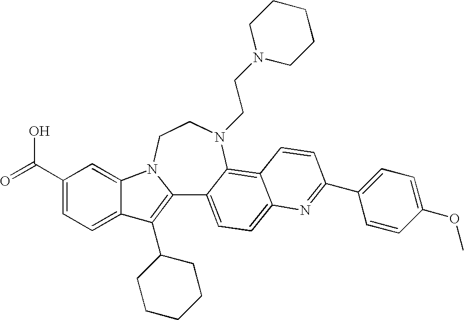 |
| 25 | 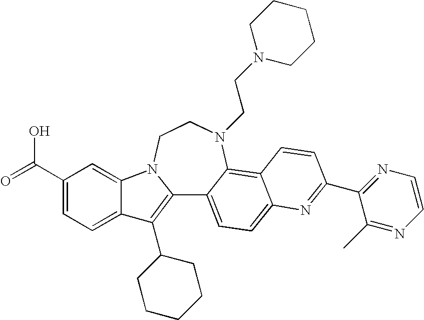 |
| 26 | 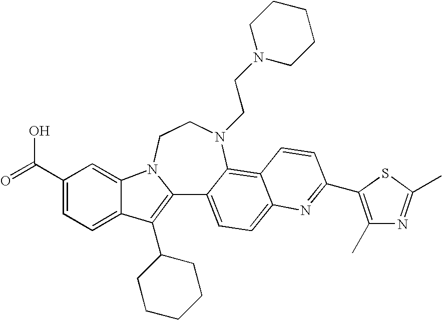 |
| 27 | 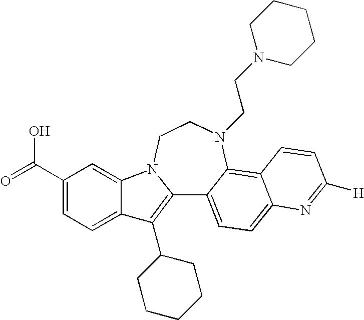 |
| 28 | 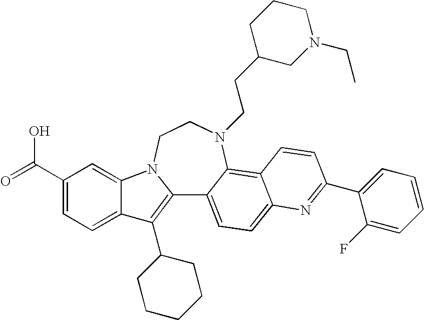 |
| 29 | 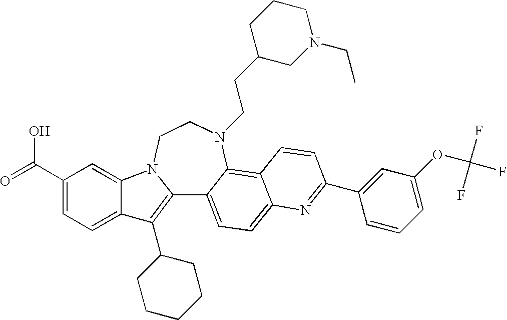 |
| 30 | 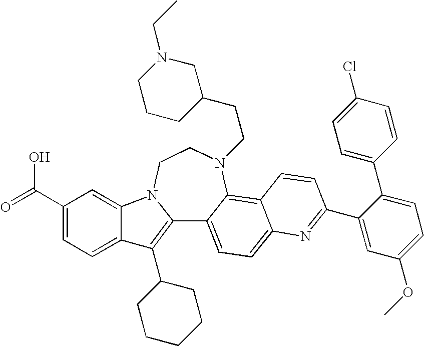 |
| 31 | 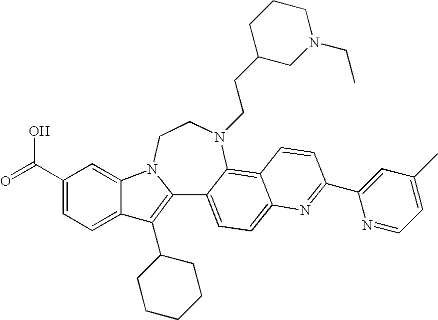 |
| 32 | 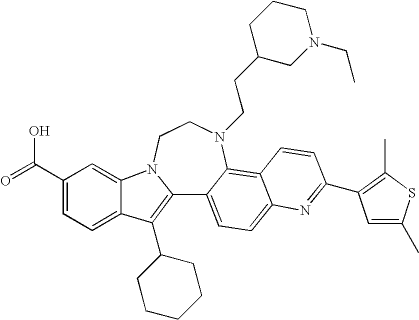 |
| 33 | 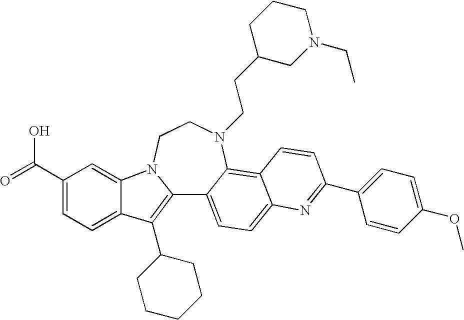 |
| 34 | 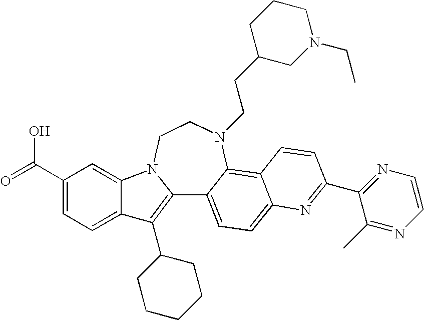 |
| 35 | 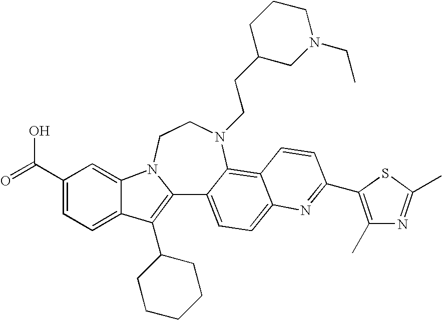 |
| 36 | 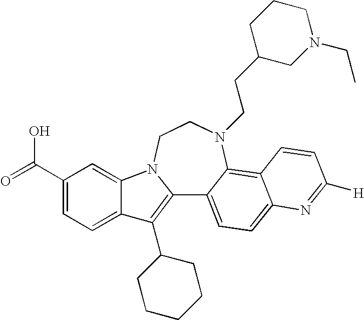 |
| 37 | 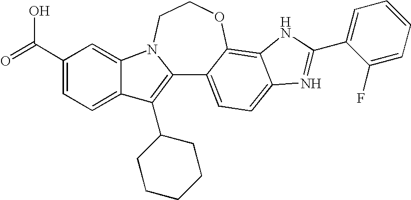 |
| 38 | 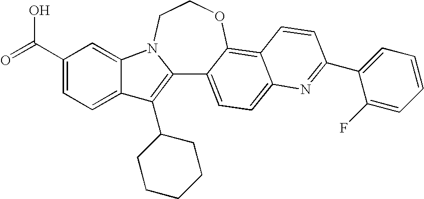 |
| 39 | 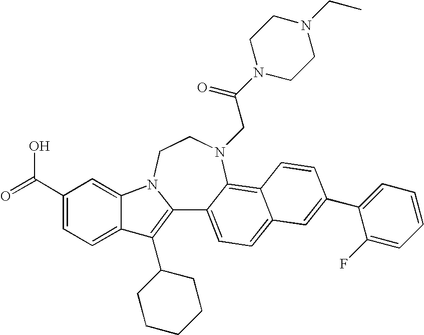 |
| 40 | 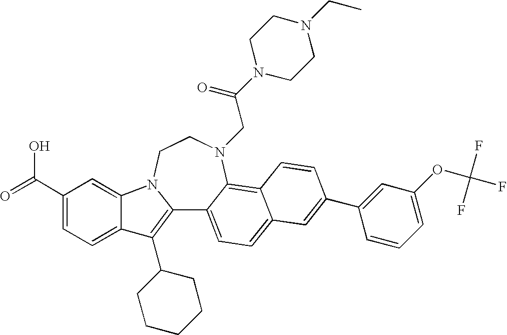 |
| 41 | 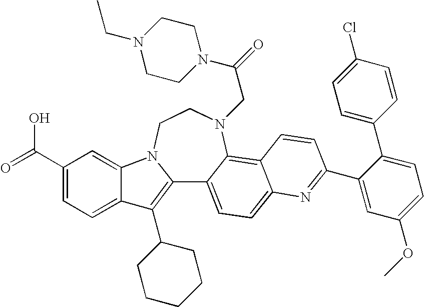 |
| 42 | 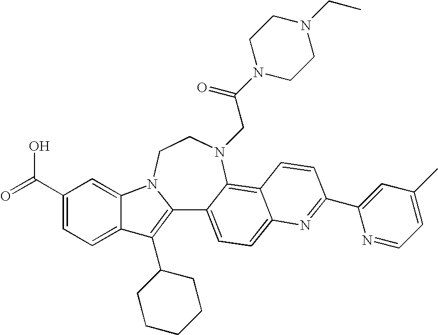 |
| 43 | 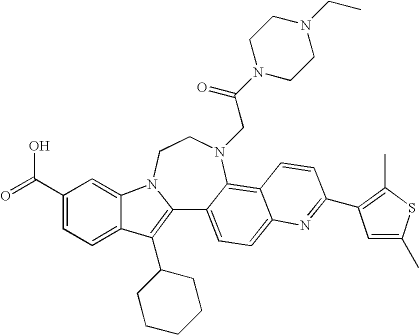 |
| 44 | 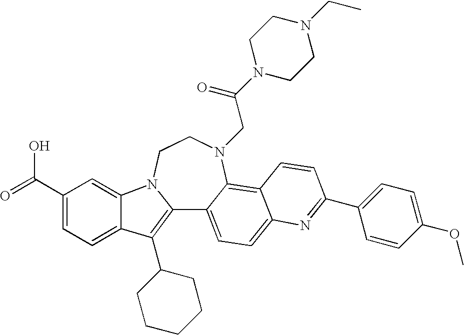 |
| 45 | 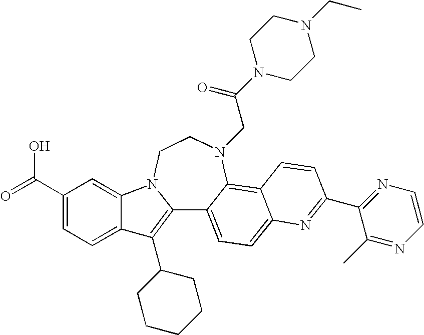 |
| 46 | 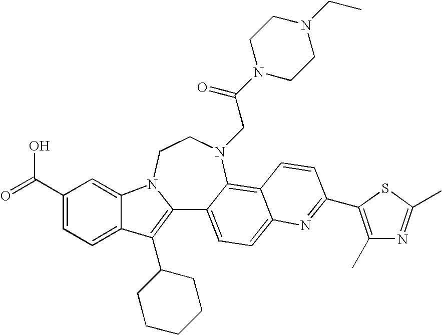 |
| 47 | 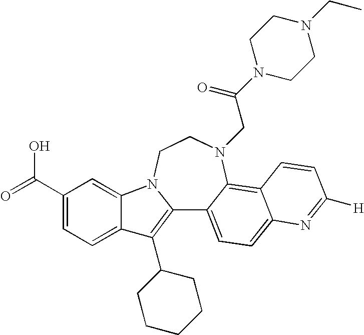 |
| 48 | 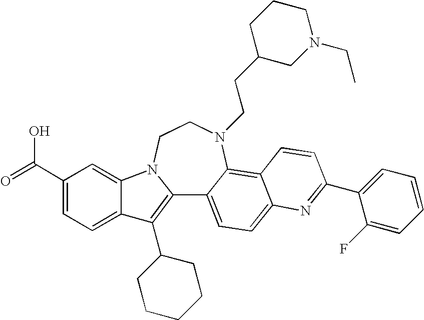 |
| 49 | 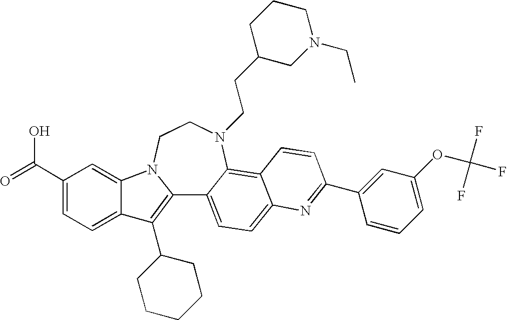 |
| 50 | 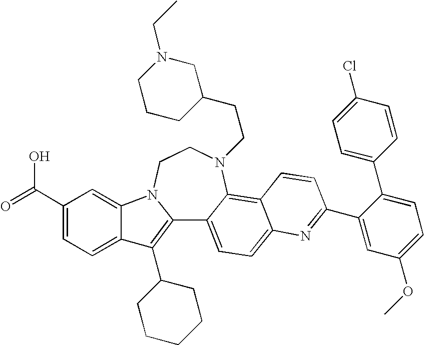 |
| 51 | 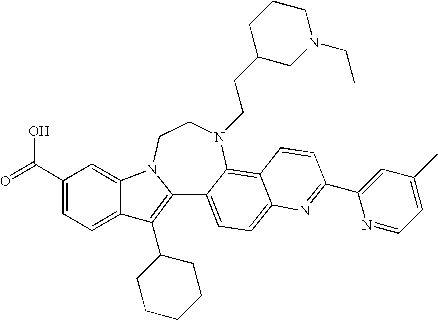 |
| 52 | 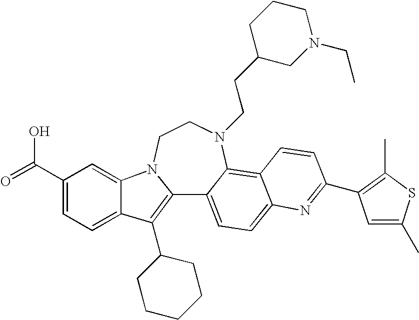 |
| 53 | 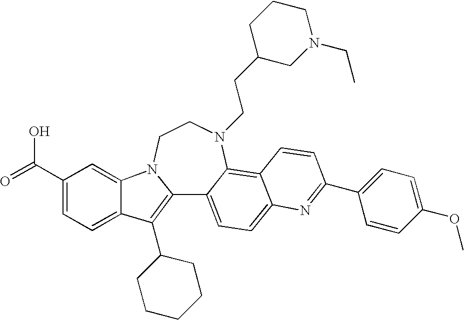 |
| 54 | 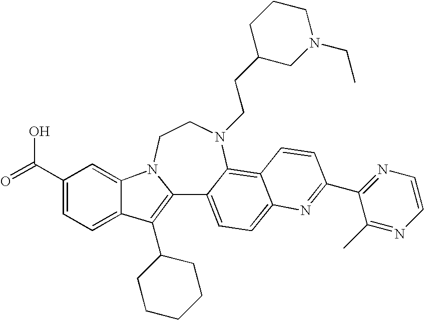 |
| 55 | 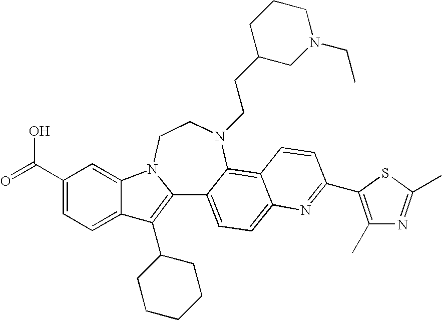 |
| 56 | 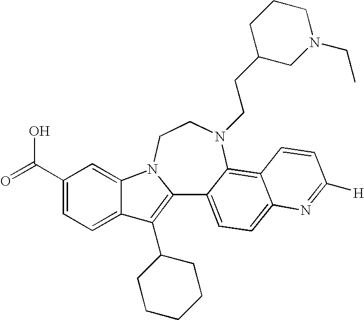 |
| 57 | 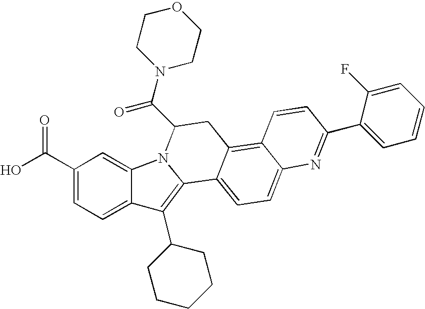 |
| 58 | 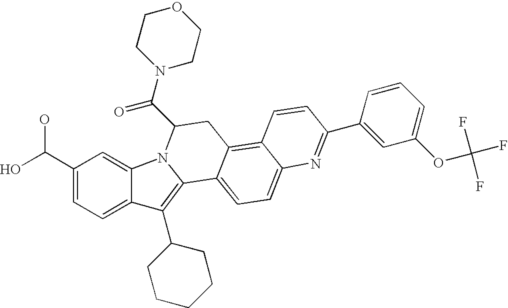 |
| 59 | 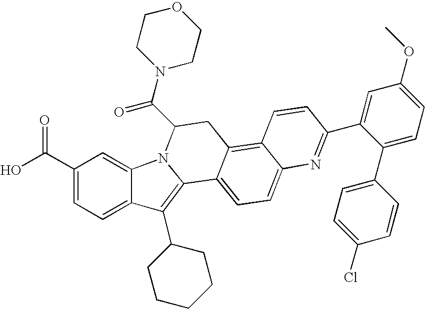 |
| 60 | 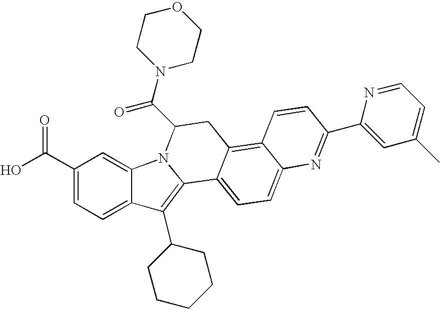 |
| 61 | 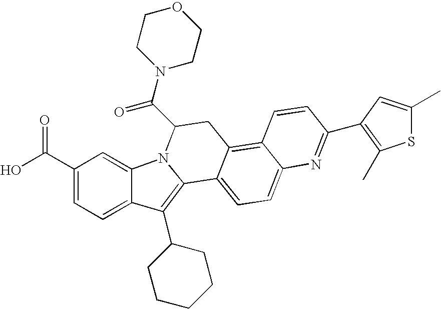 |
| 62 | 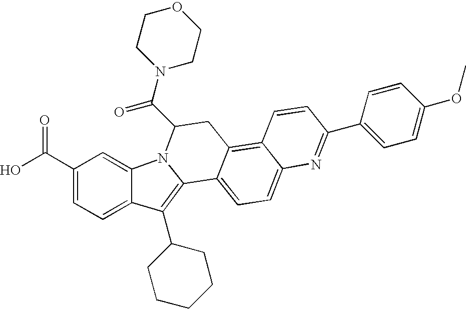 |
| 63 | 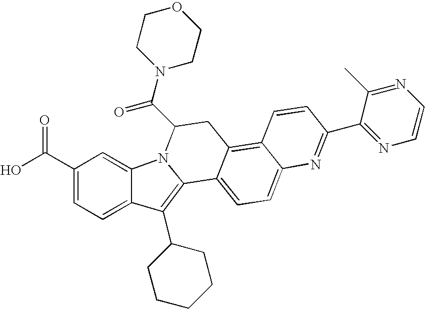 |
| 64 | 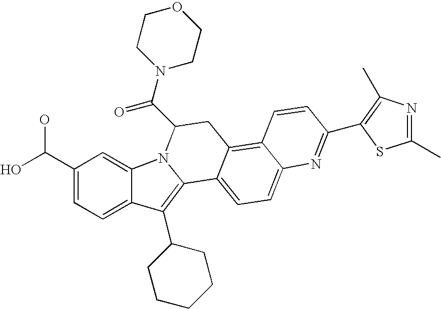 |
| 65 | 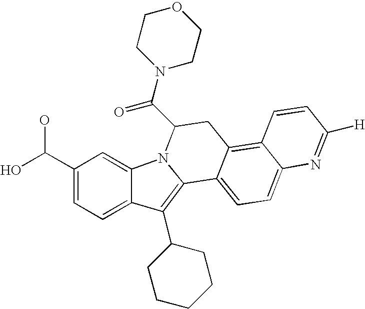 |
| 66 | 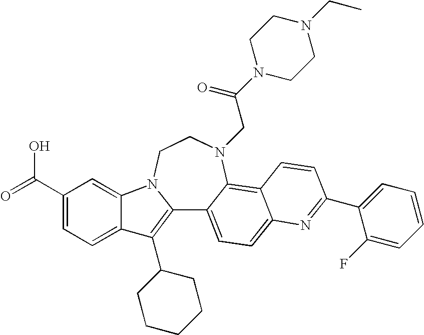 |
| 67 | 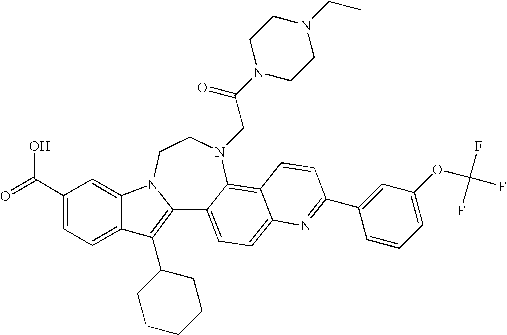 |
| 68 | 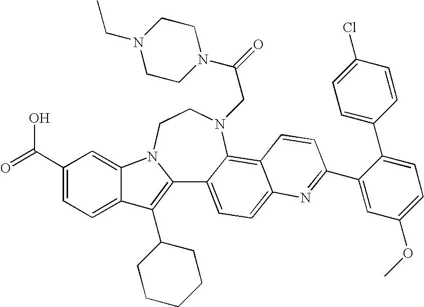 |
| 69 | 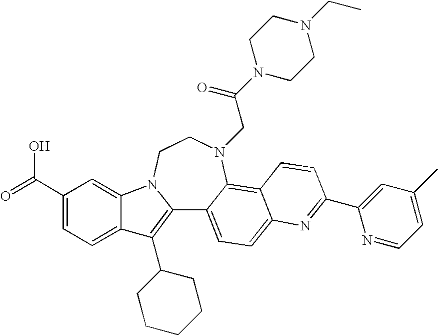 |
| 70 | 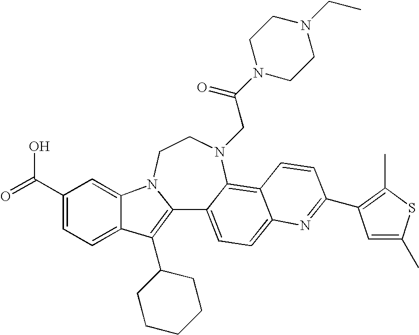 |
| 71 | 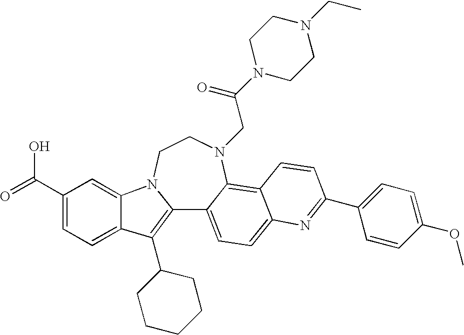 |
| 72 | 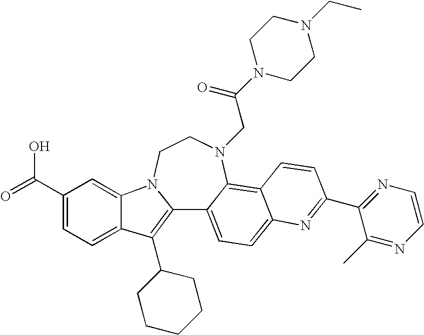 |
| 73 | 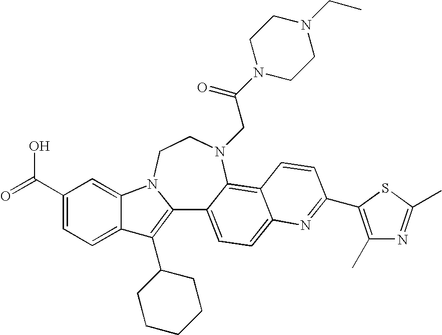 |
| 74 | 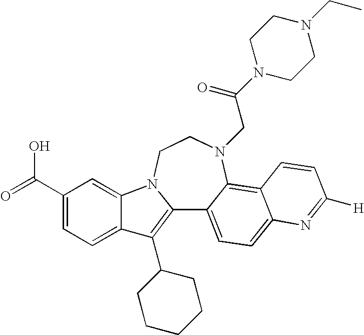 |
| 75 | 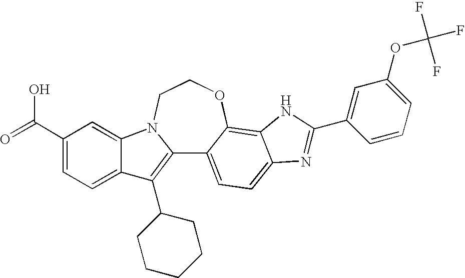 |
| 76 | 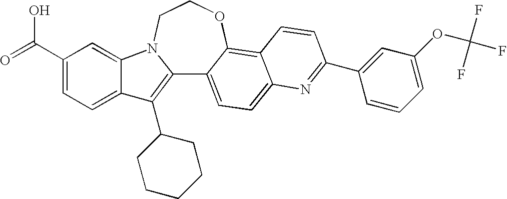 |
| 77 | 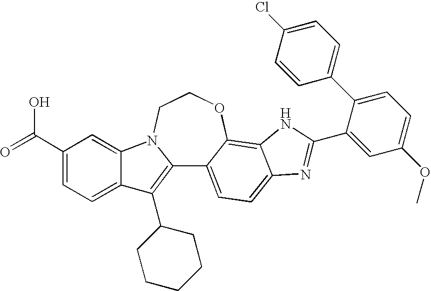 |
| 78 | 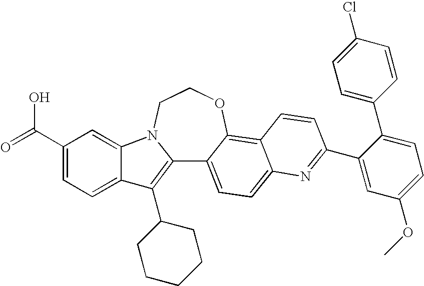 |
| 79 | 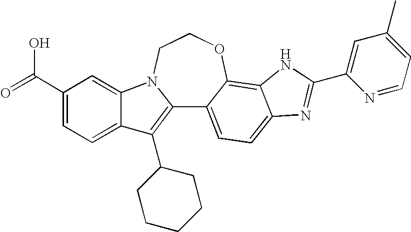 |
| 80 | 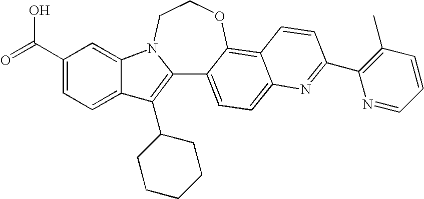 |
| 81 | 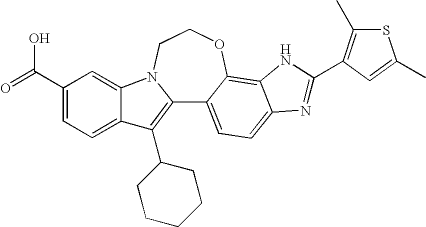 |
| 82 | 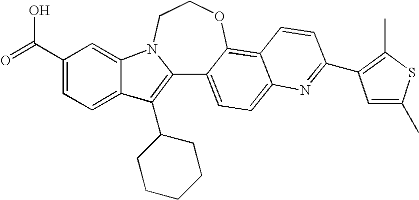 |
| 83 | 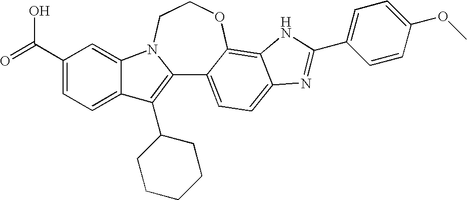 |
| 84 | 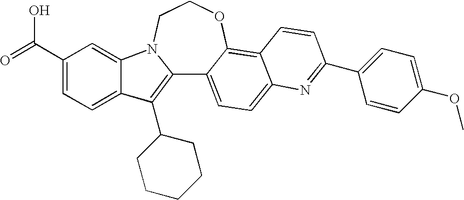 |
| 85 | 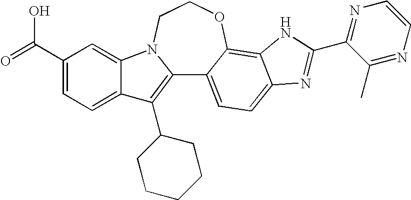 |
| 86 | 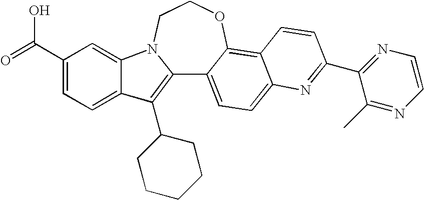 |
| 87 | 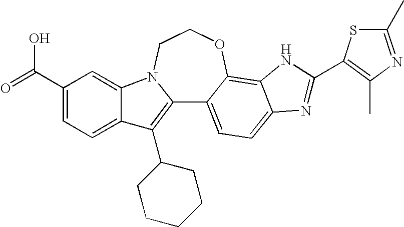 |
| 88 | 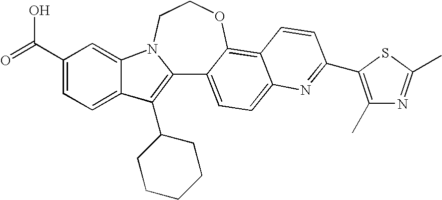 |
| 89 | 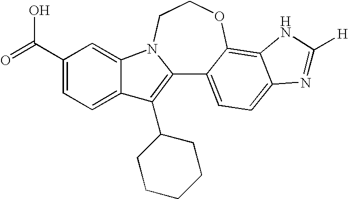 |
| 90 | 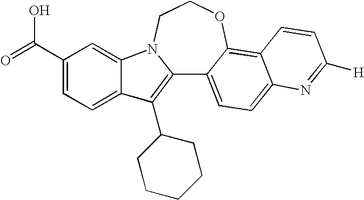 |
| 91 | 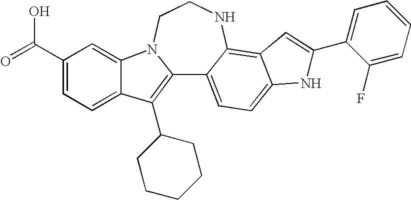 |
| 92 | 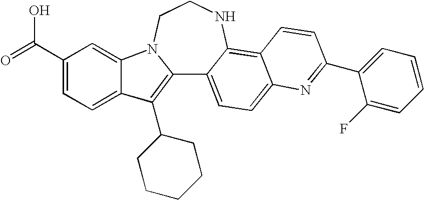 |
| 93 | 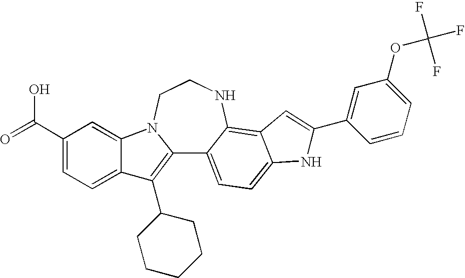 |
| 94 | 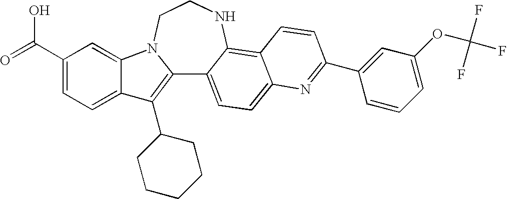 |
| 95 | 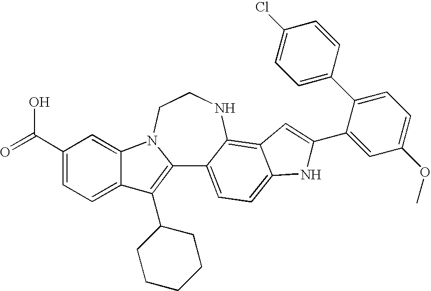 |
| 96 | 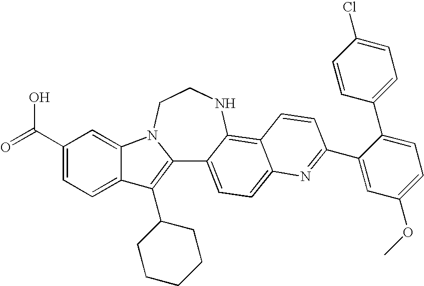 |
| 97 | 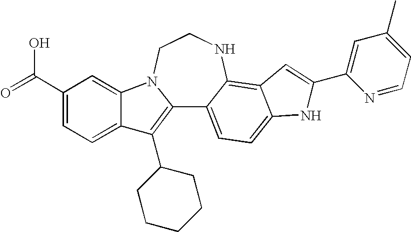 |
| 98 | 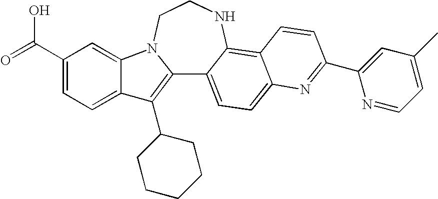 |
| 99 | 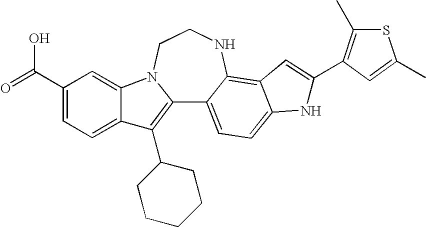 |
| 100 | 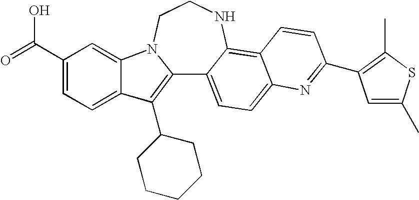 |
| 101 | 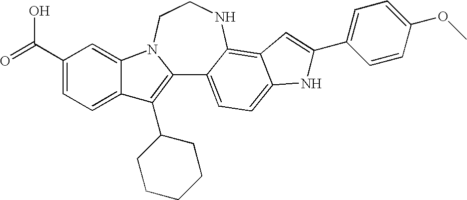 |
| 102 | 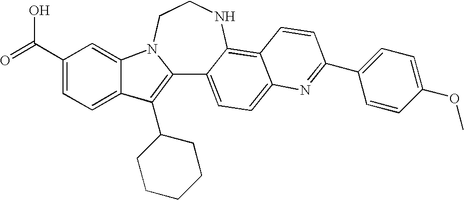 |
| 103 | 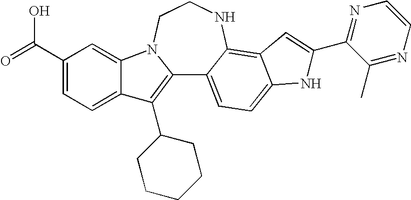 |
| 104 | 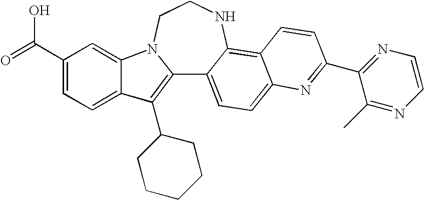 |
| 105 | 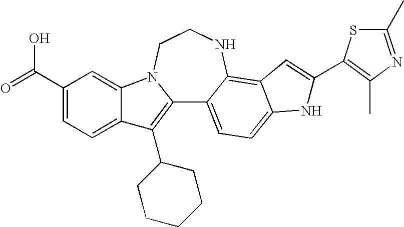 |
| 106 | 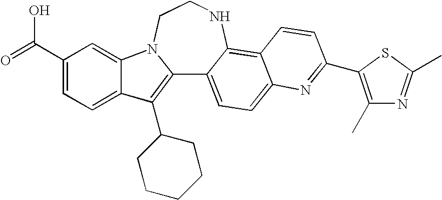 |
| 107 | 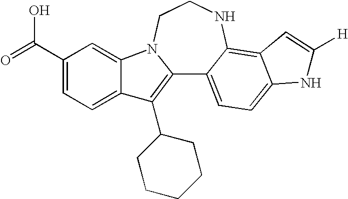 |
| 108 | 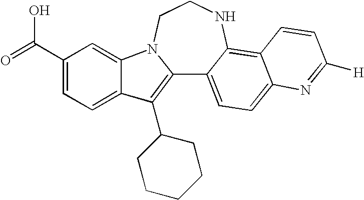 |
| 109 | 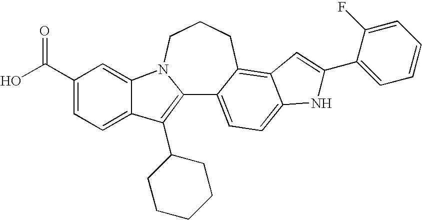 |
| 110 | 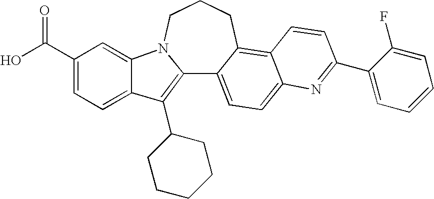 |
| 111 | 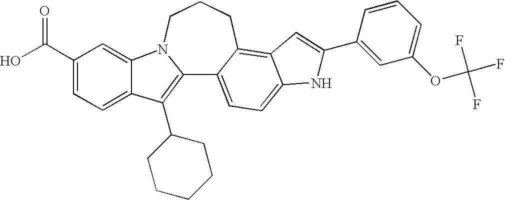 |
| 112 | 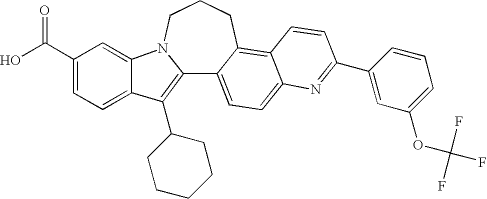 |
| 113 | 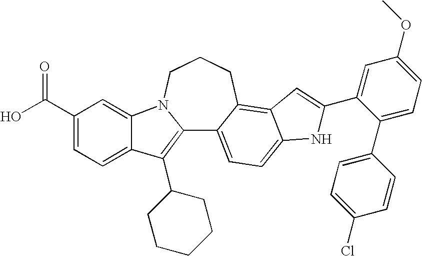 |
| 114 | 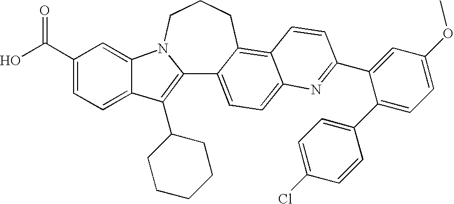 |
| 115 | 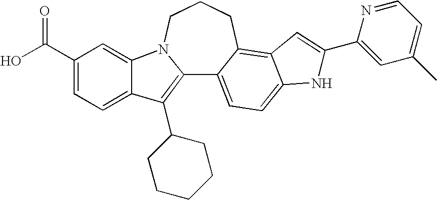 |
| 116 | 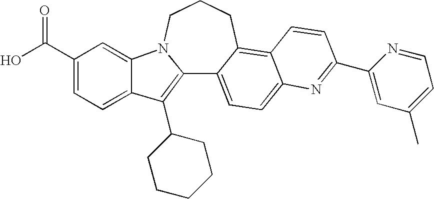 |
| 117 | 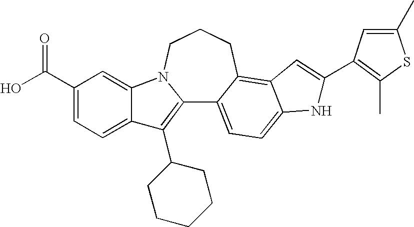 |
| 118 | 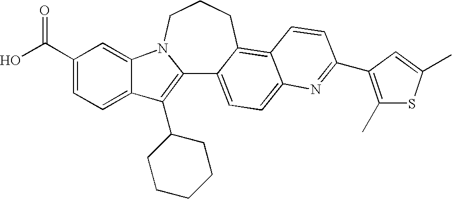 |
| 119 | 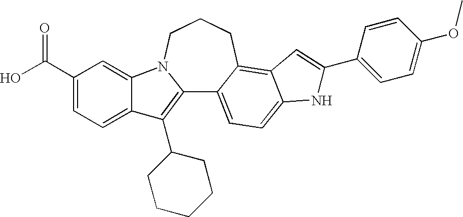 |
| 120 | 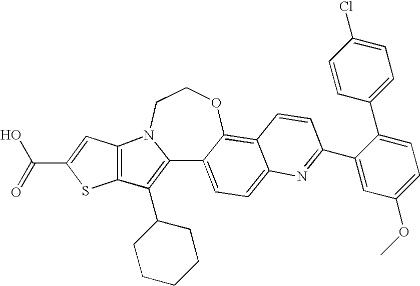 |
| 121 | 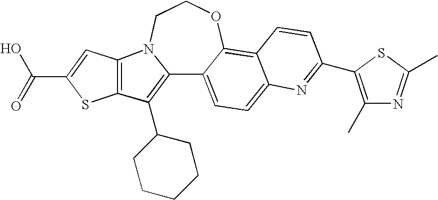 |
| 122 | 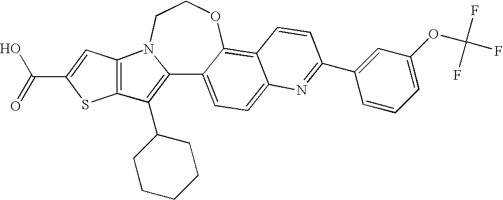 |
| 123 | 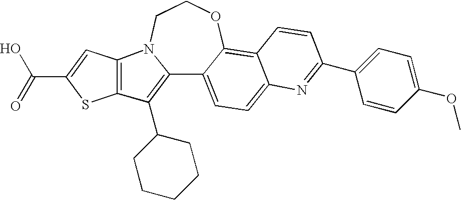 |
| 124 | 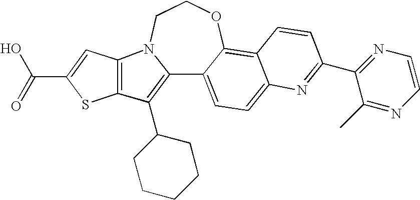 |
| 125 | 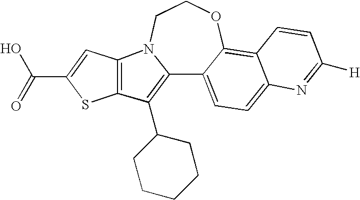 |
| 126 | 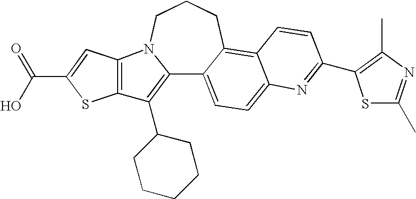 |
| 127 | 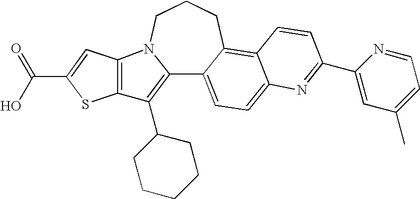 |
| 128 | 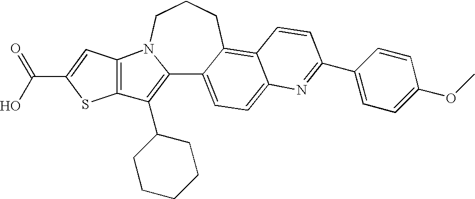 |
| 129 | 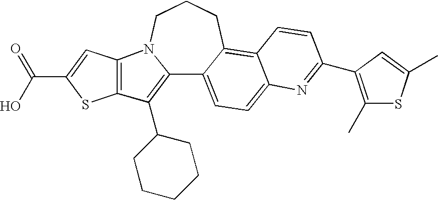 |
| 130 | 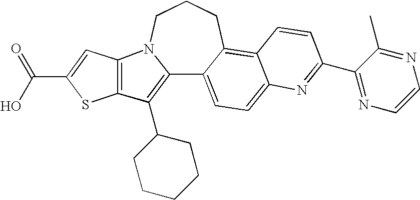 |
| 131 | 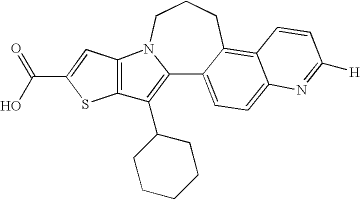 |
| 132 | 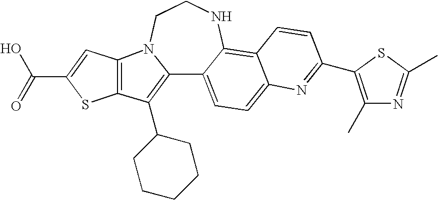 |
| 133 | 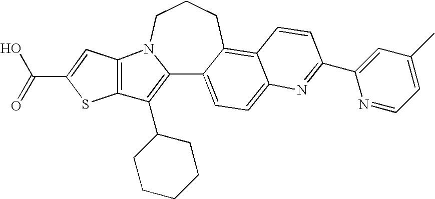 |
| 134 | 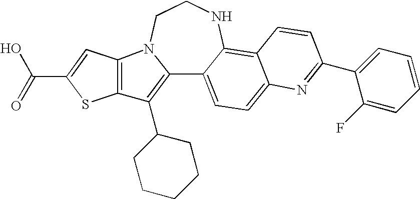 |
| 135 | 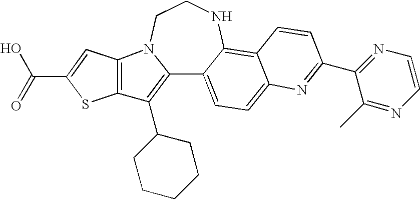 |
| 136 | 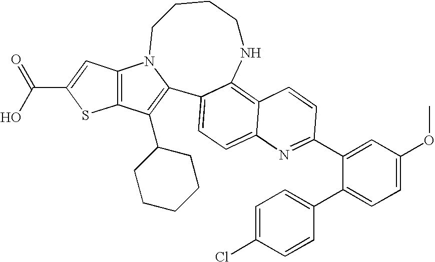 |
| 137 | 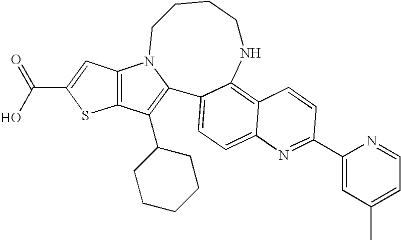 |
| 138 | 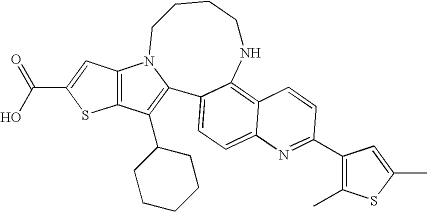 |
| 139 | 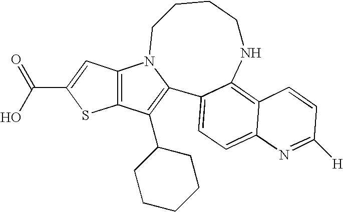 |
| 140 | 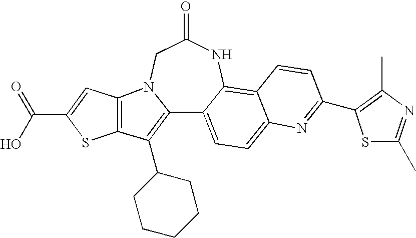 |
| 141 | 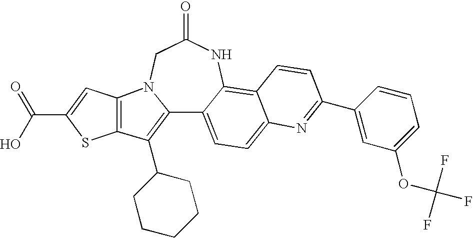 |
| 142 | 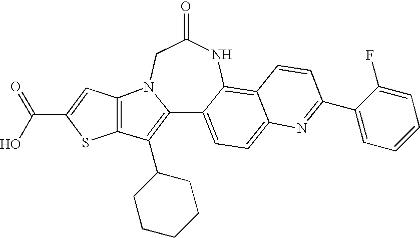 |
| 143 | 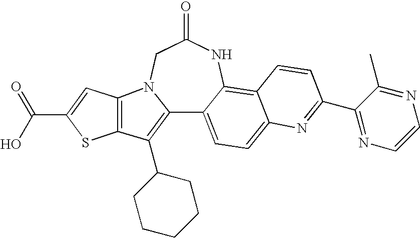 |
| 144 | 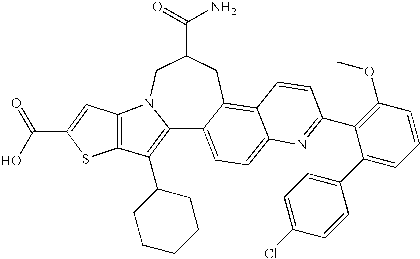 |
| 145 | 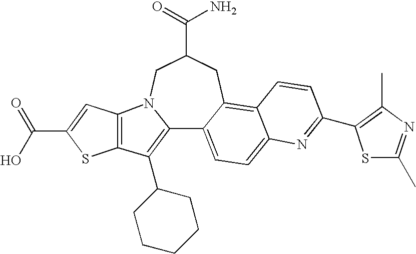 |
| 146 | 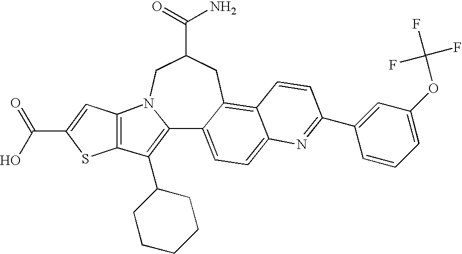 |
| 147 | 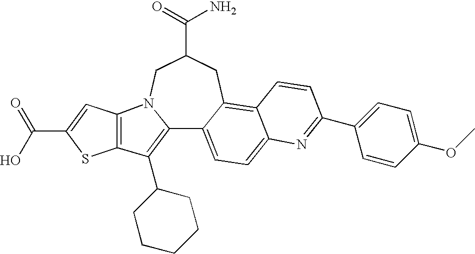 |
| 148 | 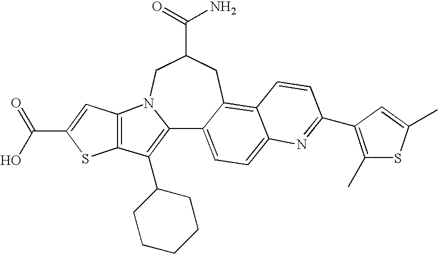 |
| 149 | 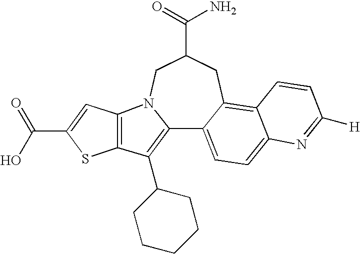 |
| 150 | 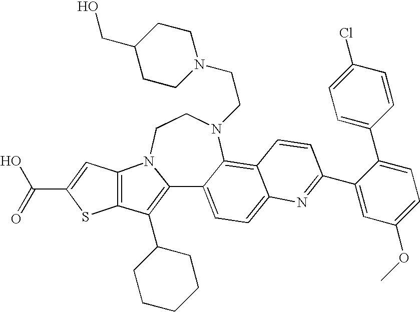 |
| 151 | 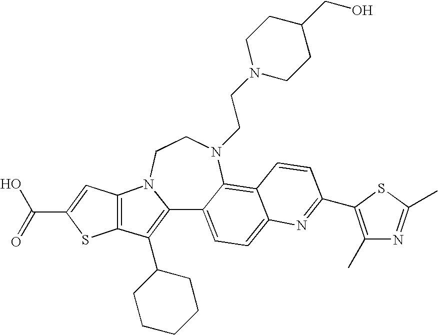 |
| 152 | 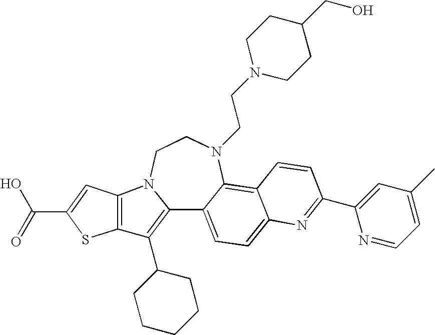 |
| 153 | 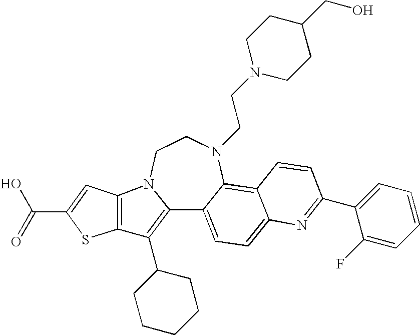 |
| 154 | 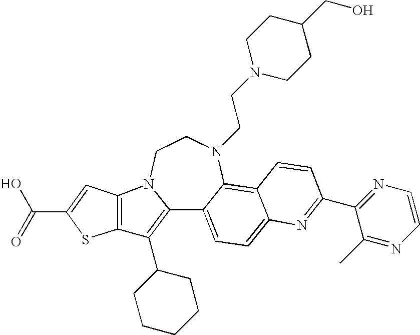 |
| 155 | 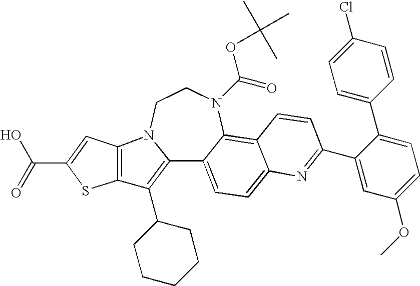 |
| 156 | 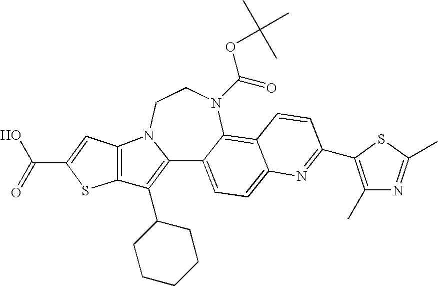 |
| 157 | 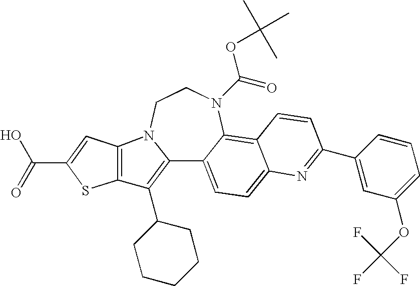 |
| 158 | 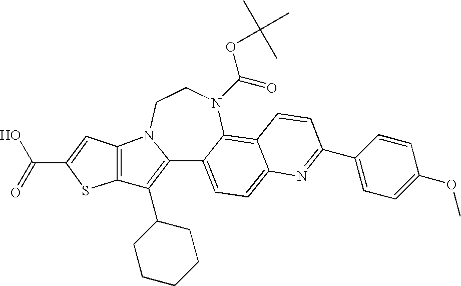 |
| 159 | 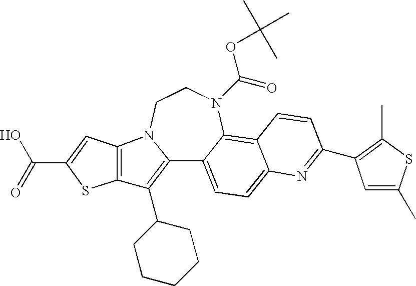 |
| 160 | 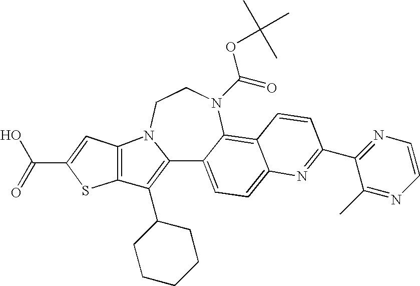 |
| 161 | 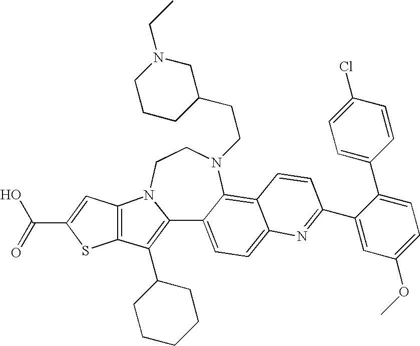 |
| 162 | 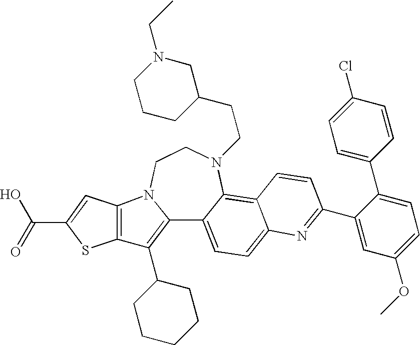 |
| 163 | 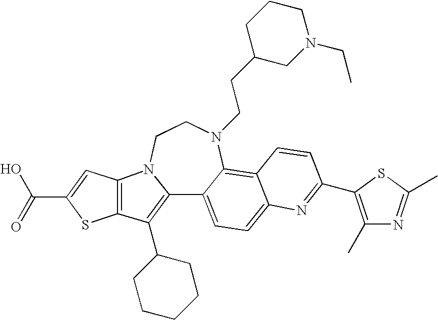 |
| 164 | 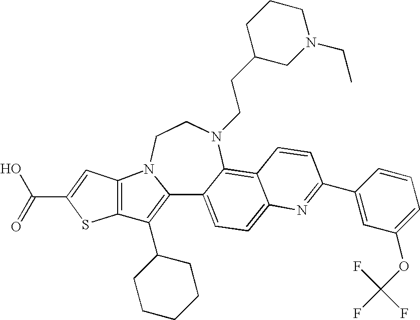 |
| 165 | 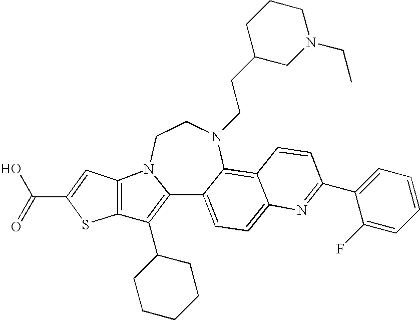 |
| 166 | 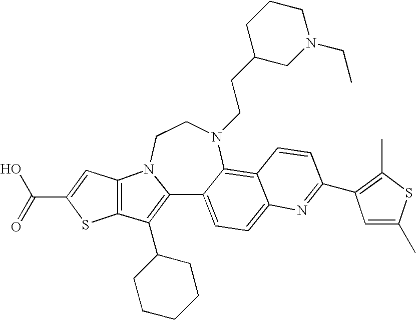 |
| 167 | 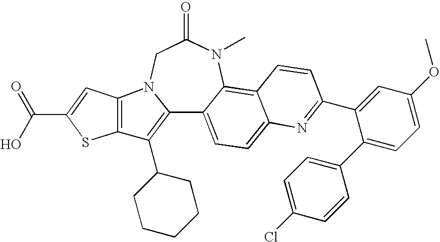 |
| 168 | 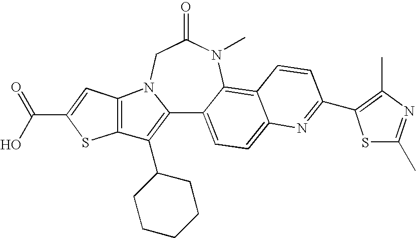 |
| 169 | 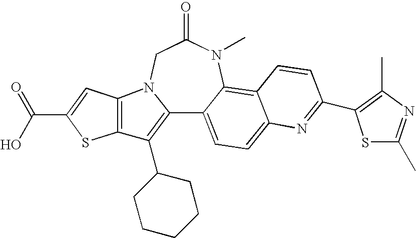 |
| 170 | 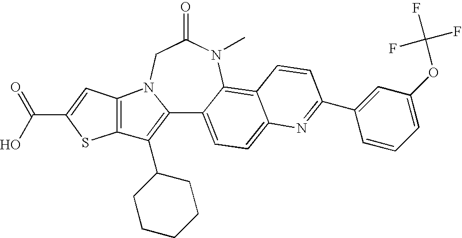 |
| 171 | 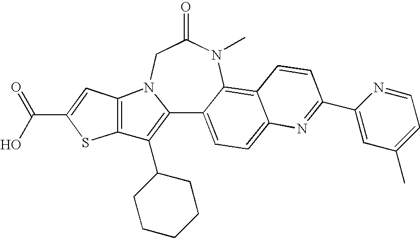 |
| 172 | 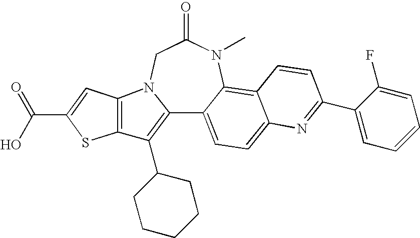 |
| 173 | 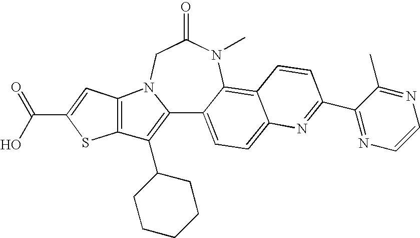 |
| 174 | 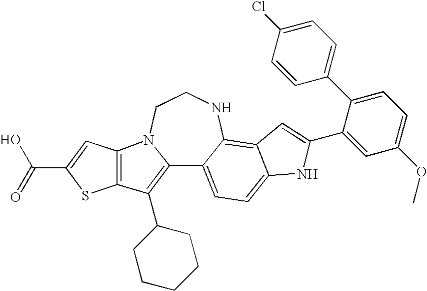 |
| 175 | 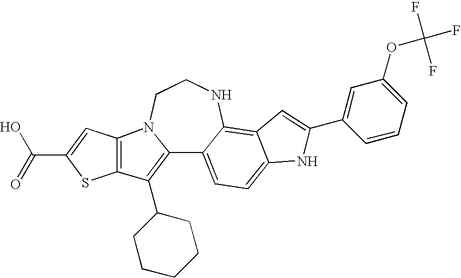 |
| 176 | 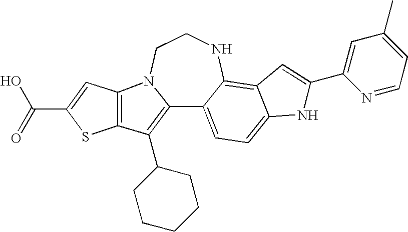 |
| 177 | 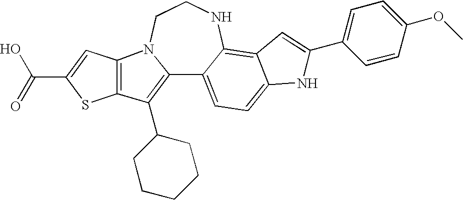 |
| 178 | 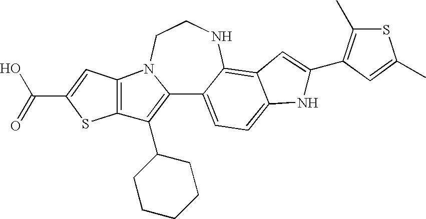 |
| 179 | 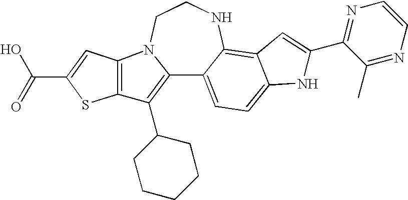 |
| 180 | 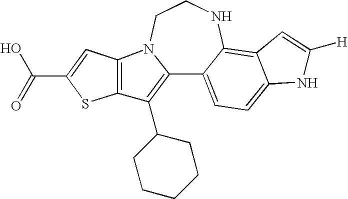 |
| 181 | 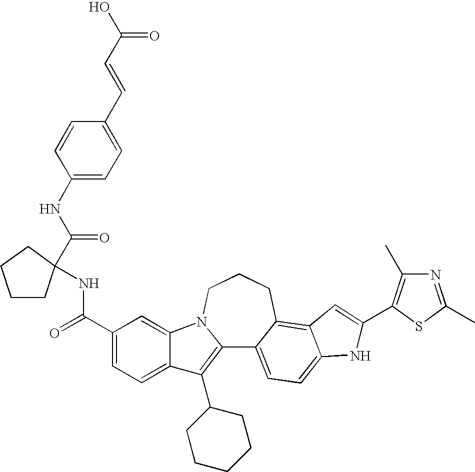 |
| 182 | 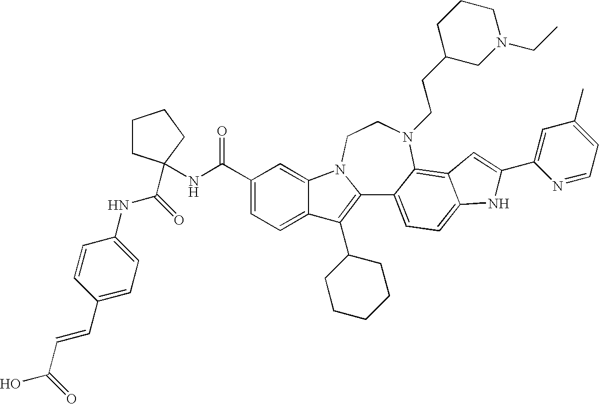 |
| 183 | 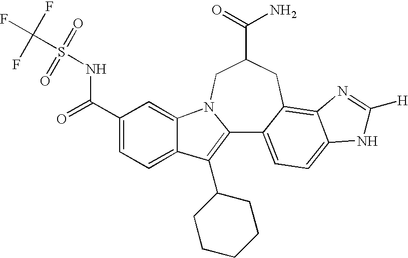 |
| 184 | 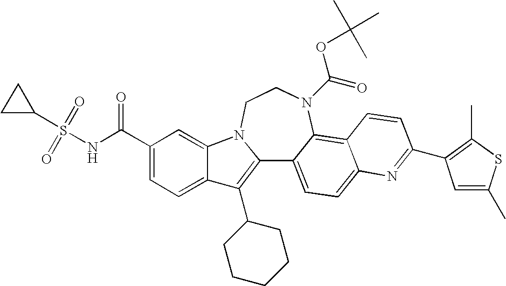 |
| 185 | 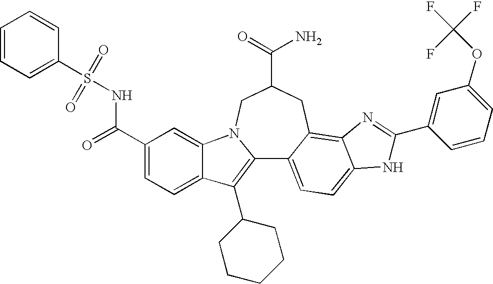 |
| 186 | 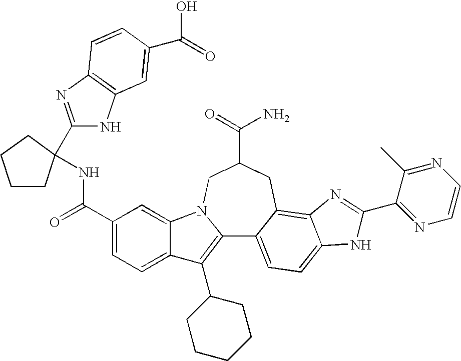 |
| 187 | 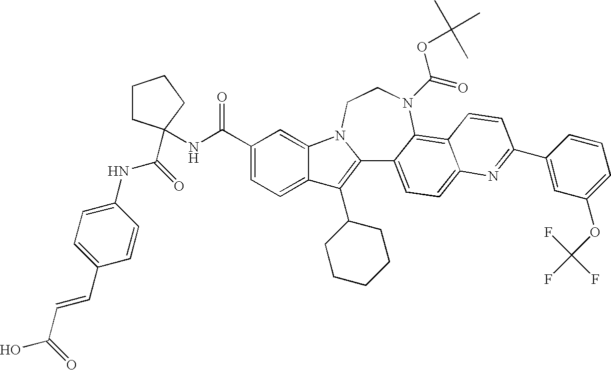 |
| 188 | 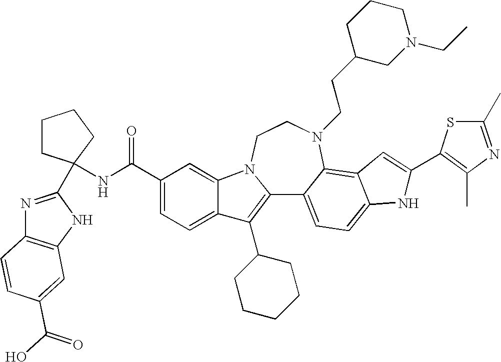 |
| 189 | 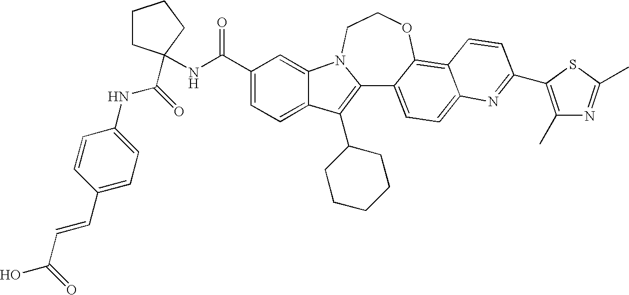 |
| 190 | 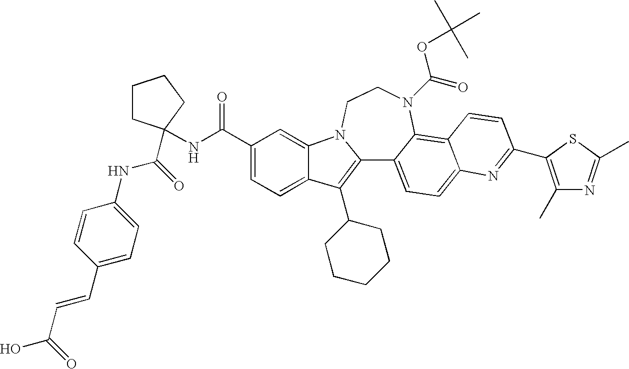 |
| 191 | 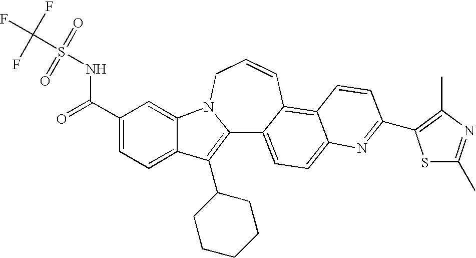 |
| 192 | 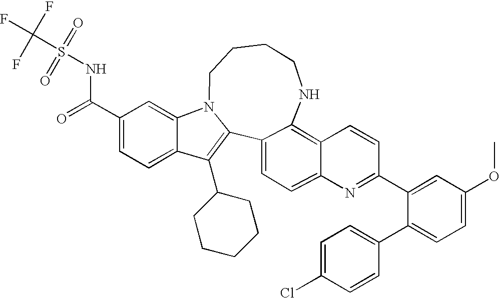 |
| 193 | 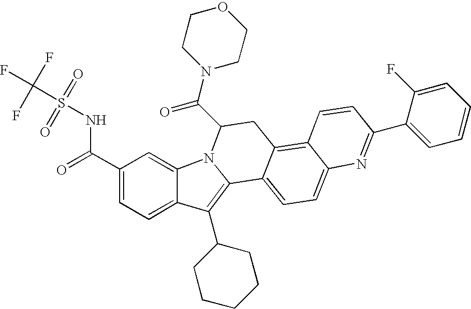 |
| 194 | 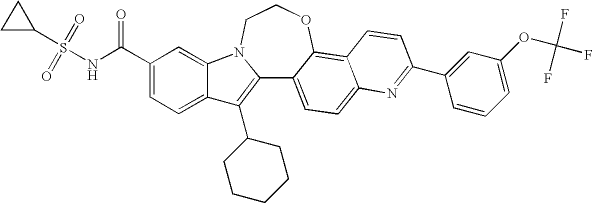 |
| 195 | 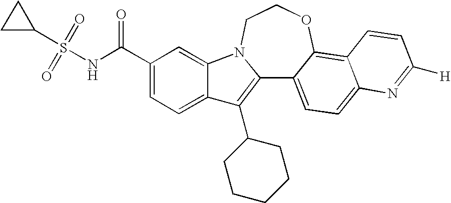 |
| 196 | 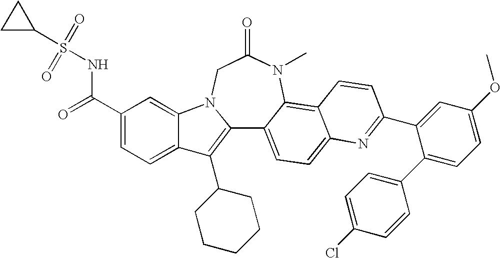 |
| 197 | 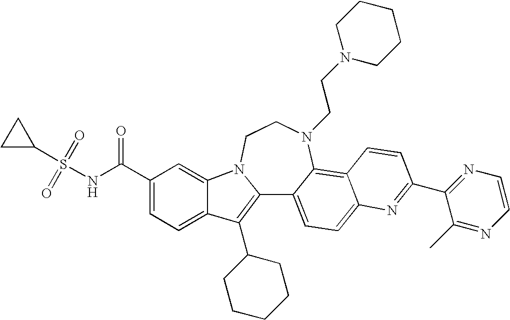 |
| 198 | 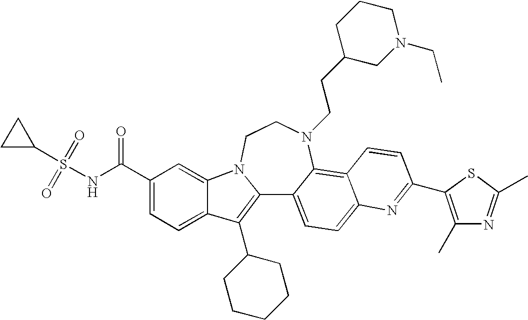 |
| 199 | 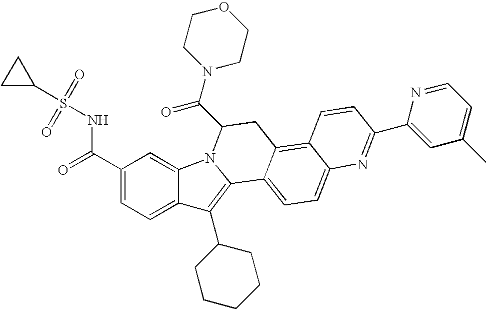 |
| 200 | 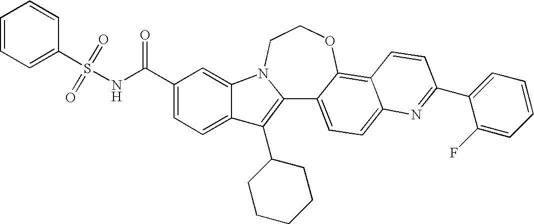 |
| 201 | 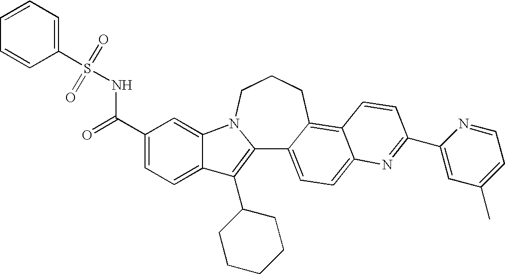 |
| 202 | 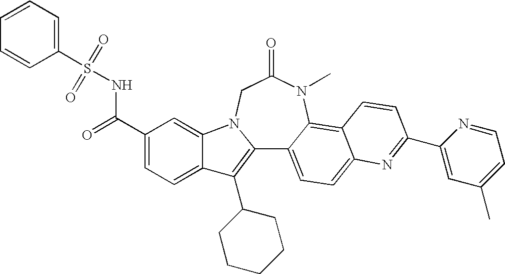 |
| 203 | 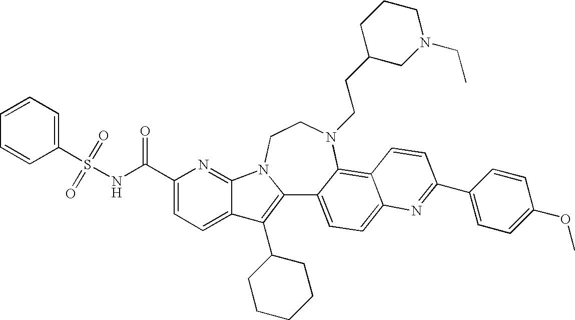 |
| 204 | 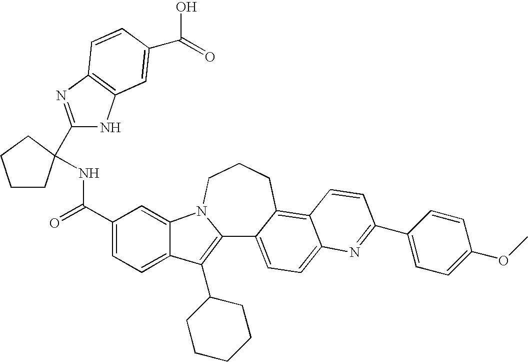 |
| 205 | 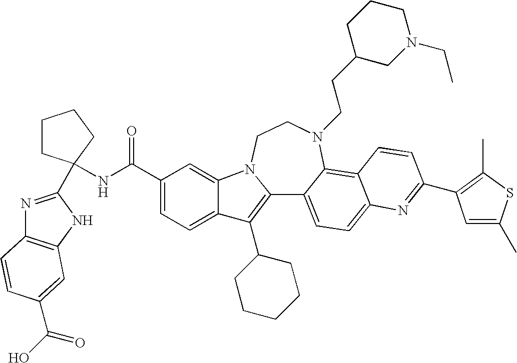 |
| 206 | 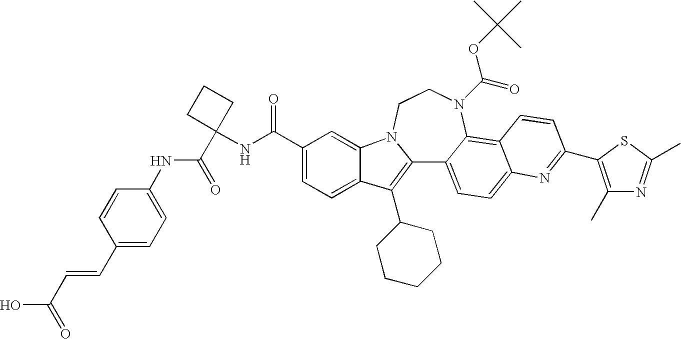 |
| 207 | 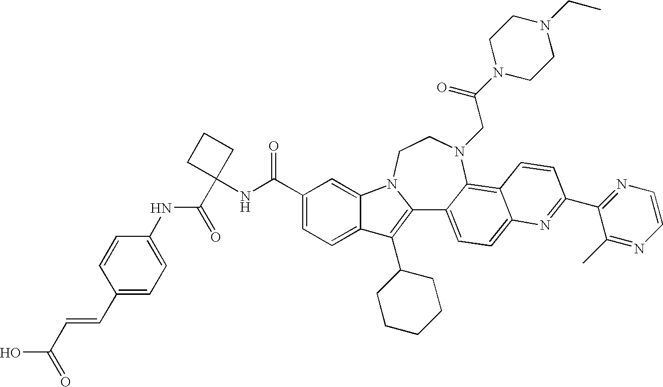 |
| 208 | 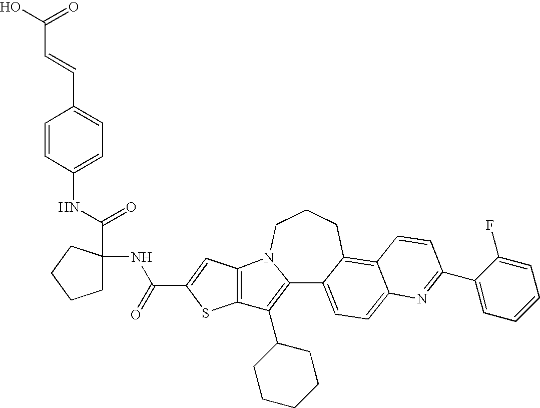 |
| 209 | 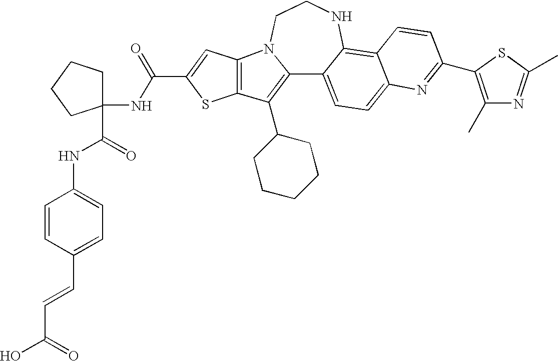 |
| 210 | 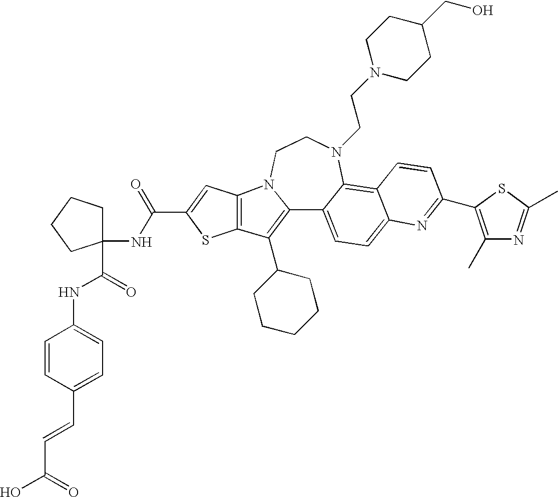 |
| 211 | 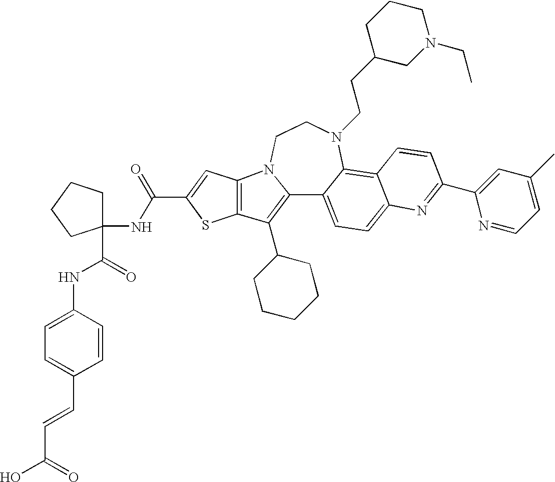 |
| 212 | 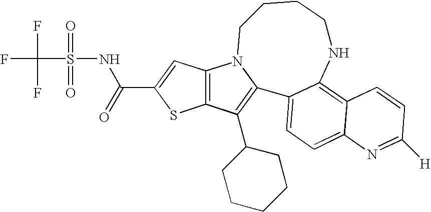 |
| 213 | 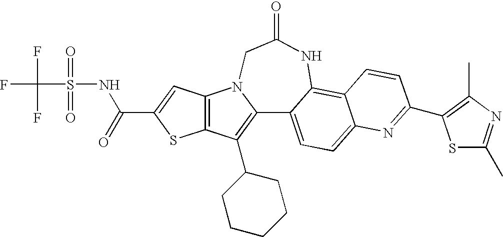 |
| 214 | 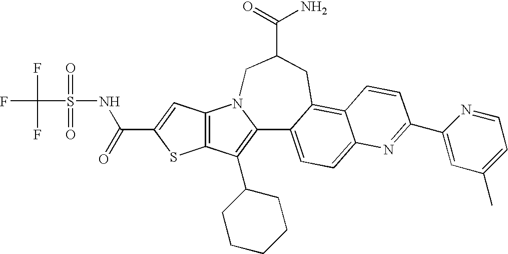 |
| 215 | 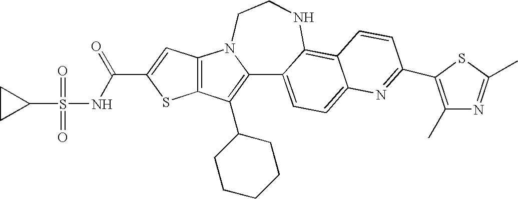 |
| 216 | 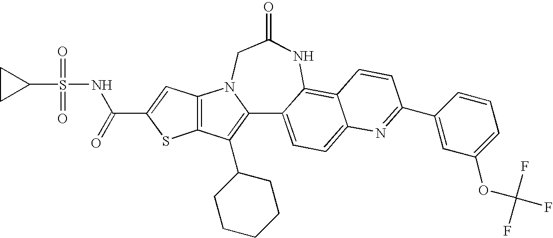 |
| 217 | 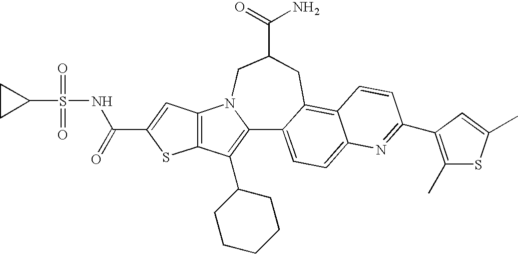 |
| 218 | 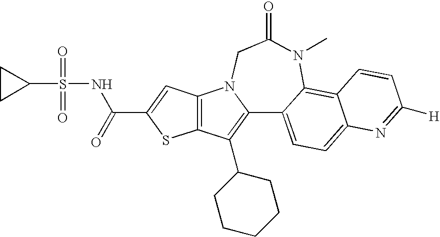 |
| 219 | 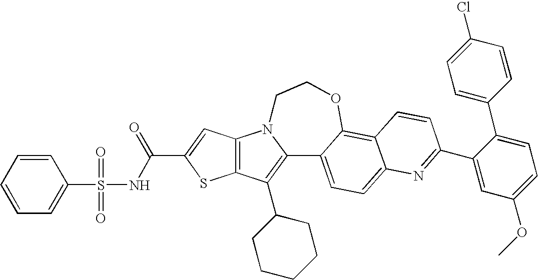 |
| 220 | 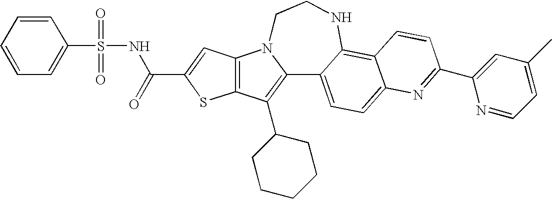 |
| 221 | 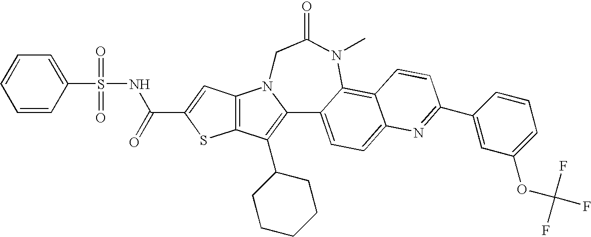 |
| 222 | 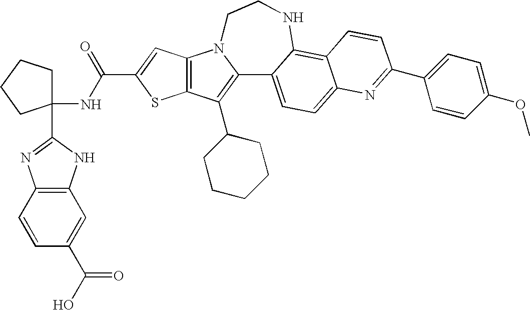 |
| 223 | 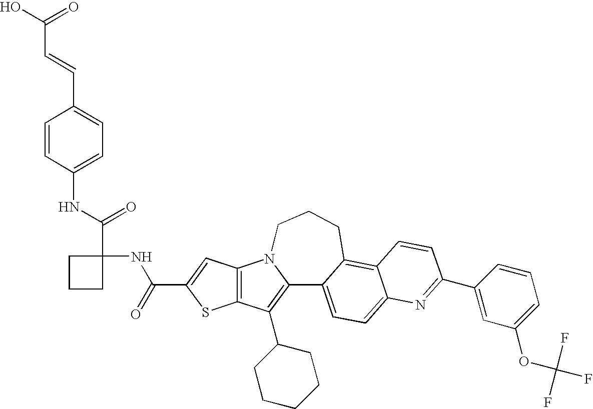 |
| 224 | 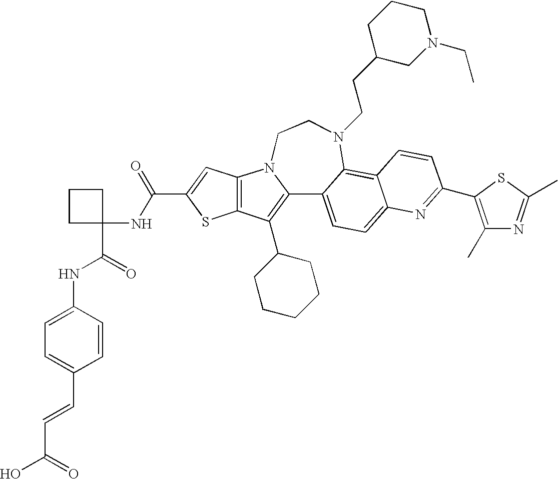 |
| 225 | 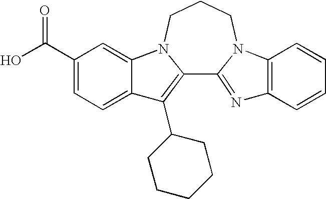 |
| 226 | 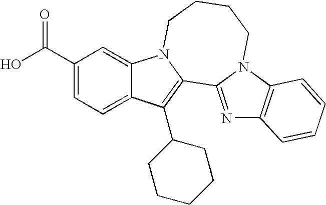 |
| 227 | 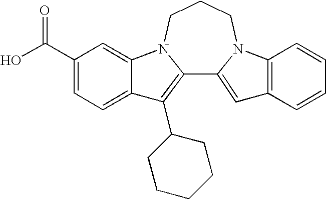 |
| 228 | 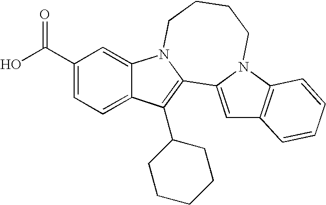 |
| 229 | 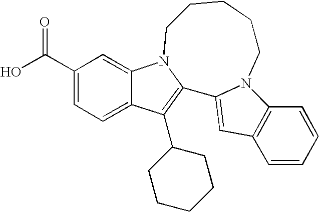 |
| 230 | 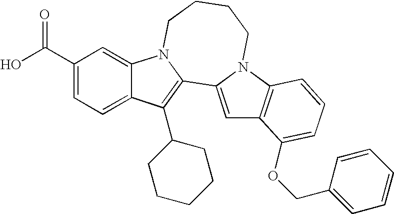 |
| 231 | 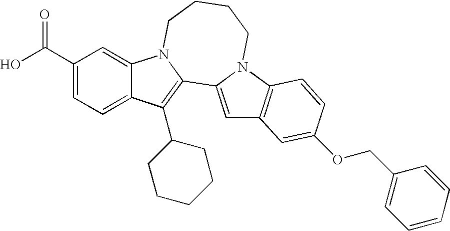 |
| 232 | 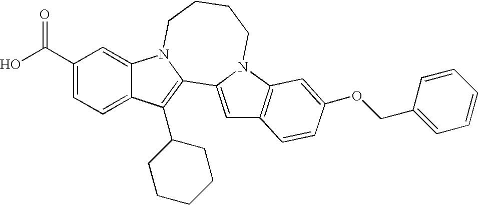 |
| 233 | 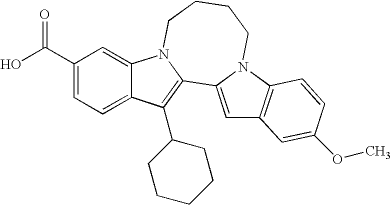 |
| 234 | 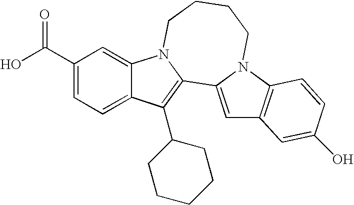 |
| 235 | 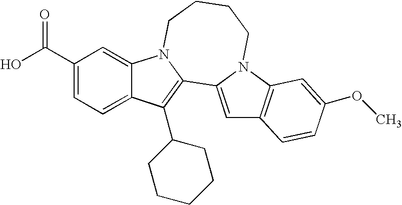 |
| 236 | 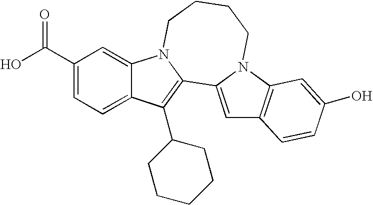 |
| 237 | 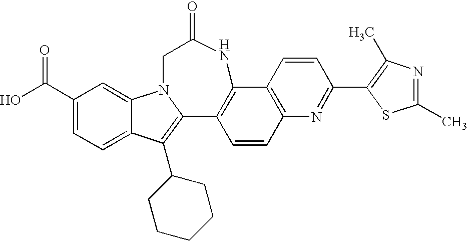 |
| 238 | 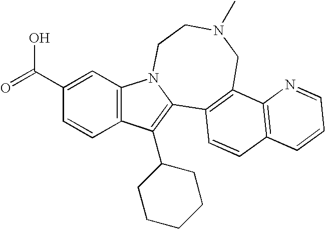 |
| 239 | 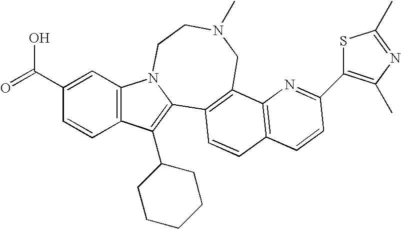 |
| 240 | 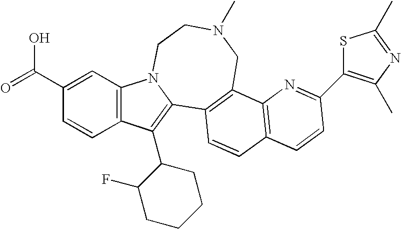 |
| 241 | 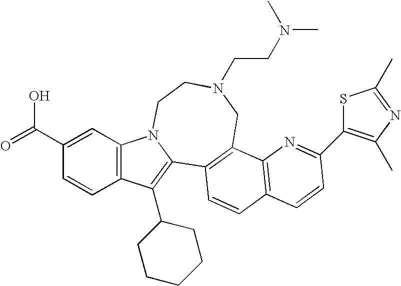 |
| 242 | 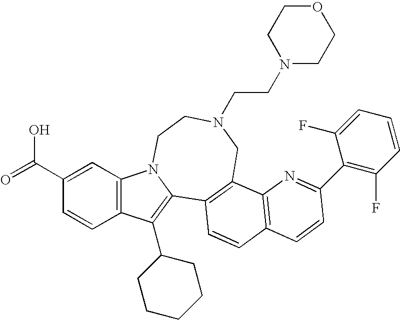 |
| 243 | 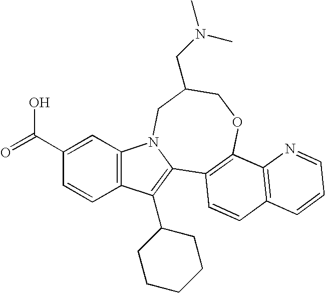 |
| 244 | 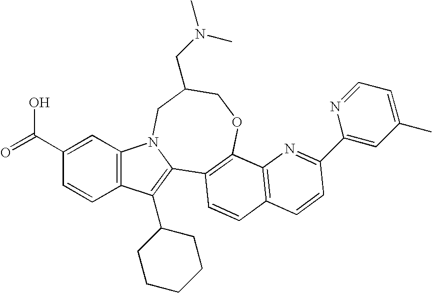 |
| 245 | 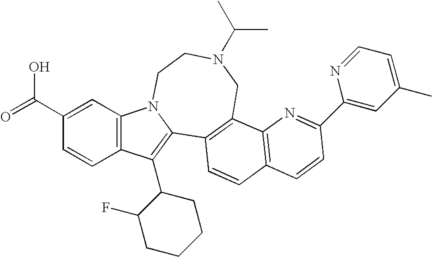 |

The present invention further provides metabolites of any of compounds of Formula (A), (I), (II), (III), (IV), (Ia)-(If), (IIa)-(IIf), (IVa) or the compounds in Table 1. In some aspects, the metabolic is an oxide.

This invention is also directed to pharmaceutical compositions comprising a pharmaceutically acceptable diluent and a therapeutically effective amount of one of the compounds described herein or mixtures of one or more of such compounds.

This invention is further directed to methods for treating a viral infection mediated at least in part by a virus in the Flaviviridae family of viruses, such as HCV, in mammals which methods comprise administering to a mammal, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds. In another aspect, present invention provides for use of the compounds of the invention for the preparation of a medicament for treating or preventing said infections. In other aspects the mammal is a human.

In yet another embodiment of the invention, methods of treating or preventing viral infections in mammals are provided where in the compounds of this invention are administered in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. Preferably, the additional agent active against HCV is interferon-alpha or pegylated interferon-alpha alone or in combination with ribavirin or viramidine.

General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

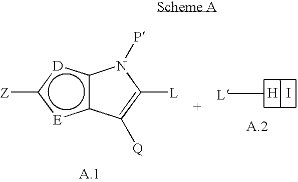

In one embodiment, the compounds of the invention may generally be prepared via a transition metal catalyzed cross-coupling reaction as shown above in Scheme A where L and L′ are suitable cross-coupling substituents, P′ is hydrogen, a nitrogen protecting group and Z, D, E, Q, ring H, and ring I are as previously defined. Typically, one of L or L′ is a Sn, B, Zr, or Zn based metal (e.g. —BOH₂, Sn(CH₃)₃, etc.) and the other of L or L′ is a leaving group such as halogen or sulfonate. Suitable halogens and sulfonates include Cl, Br, I, —OSO₂CF₃, and —OSO₂CH₃. Suitable transition metal catalysts include Pd and Ni based catalysts (e.g. Pd(PPh₃)₂Cl₂, Pd[P(Ph₃)]₄, etc.). In one embodiment, one of A.1 or A.2 has L is —B(OH)₂ and is prepared by treating a compound of A.1 or A.2 where L or L′ is halogen with an excess of bis(neopentylglycolato)diboron in the presence of a catalytic amount of triphenylphosphine palladium(II) dichloride. The resulting boronic acid is the coupled with the other of A.1 or A.2 where L is halogen or a sulfonate under Suzuki coupling conditions. Suitable coupling conditions include reaction of A.1 and A.2 in refluxing methanol containing Pd[P(Ph)₃]₄ and NaHCO₃ for 10 to 20 hours. The coupled product can then be further modified to yield the compounds of the invention using methods that will be apparent to one of skill in the art as illustrated in the following Schemes.

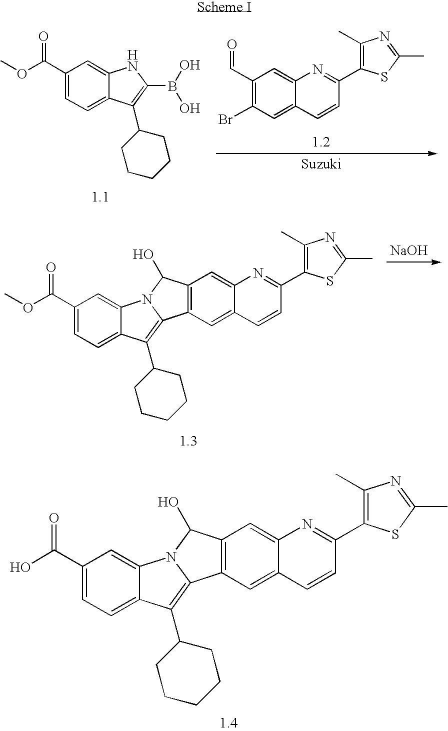

For illustrative purposes, Scheme 1 shows the synthesis of compounds where Z is COOH, Q is cyclohexyl, rings H and I are as depicted, and T is —CH₂— substituted with OH. Compound 1.4 is formed following Suzuki coupling of 1.1 and 1.2 to form the five membered central ring and saponification of 1.3 under basic conditions such as with NaOH.

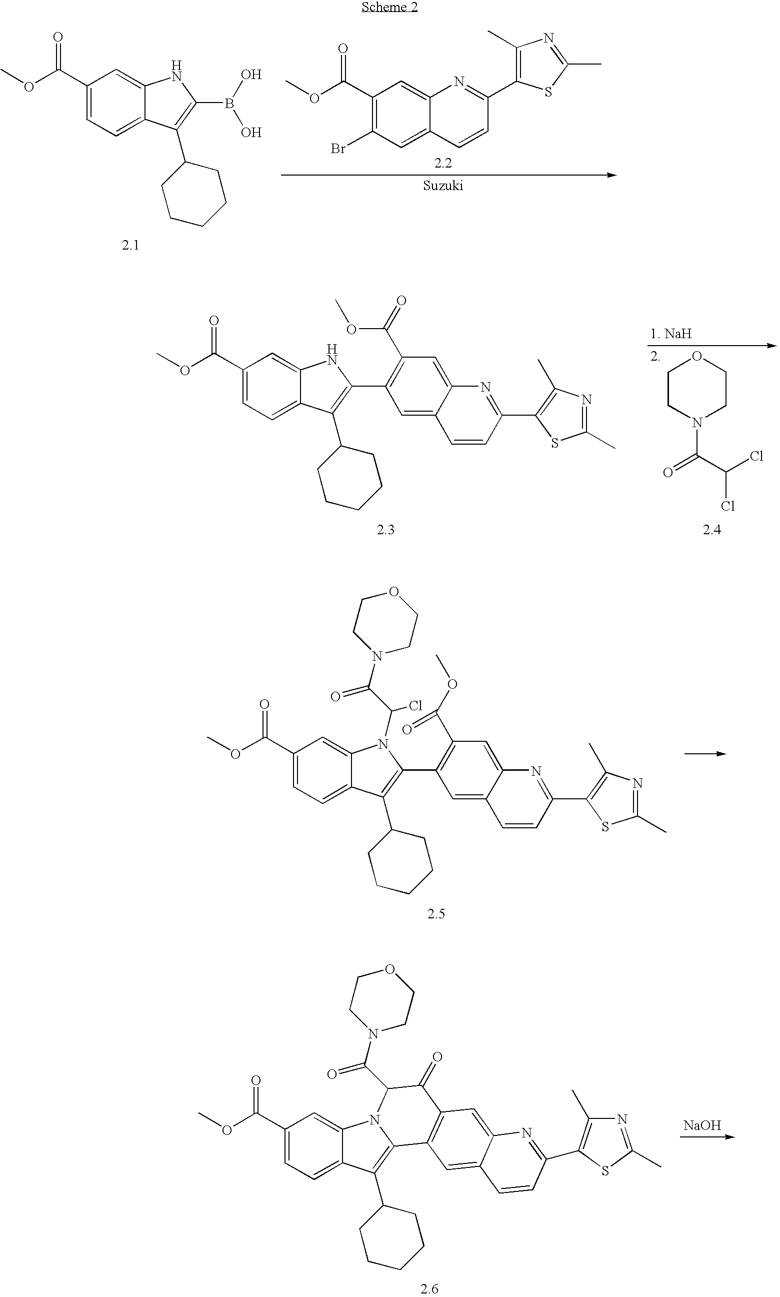

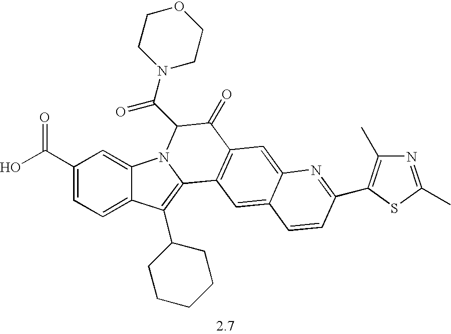

For illustrative purposes, Scheme 2 shows the synthesis of compounds where Z is COOH, Q is cyclohexyl, rings H and I are as depicted, and T is a substituted alkylene that forms a central six member ring. Suzuki coupling of 2.1 with 2.2 gives the coupled indole 2.3 that is then deprotonated with a base such as NaH and alkylated with acyl dichloride 2.4. The resulting chloride 2.5 is metallated such as by treatment with n-BuLi to effect intramolecular ring closure to give 2.6. Saponification of the resulting compound under basic conditions give 2.7.

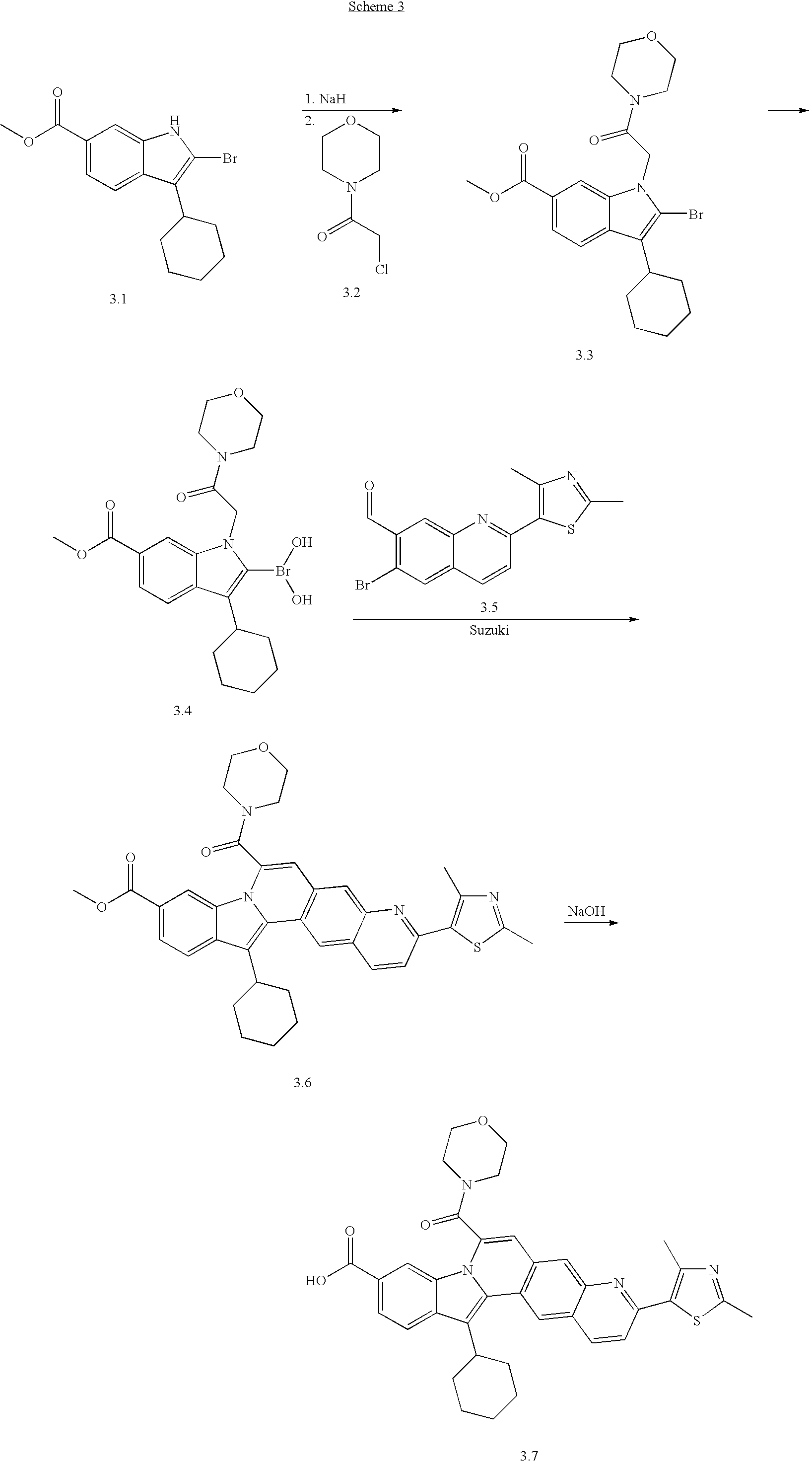

For illustrative purposes, Scheme 3 shows the synthesis of compounds where Z is COOH, Q is cyclohexyl, rings H and I are as depicted, and T is a substituted alkylene having a cis-double bond that forms a central six member ring. Indole 3.1 is deprotonated and alkylated with 3.2 to give bromide 3.3 that is then converted to boronic acid 3.4 upon treatment with an excess of bis(neopentylglycolato)diboron in the presence of a palladium(0) catalyst such as triphenylphosphine palladium(II) dichloride. Suzuki coupling of 3.4 with 3.5 followed by an intramolecular aldol condensation gives the coupled indole 3.6. Saponification of the resulting compound under basic conditions give 3.7.

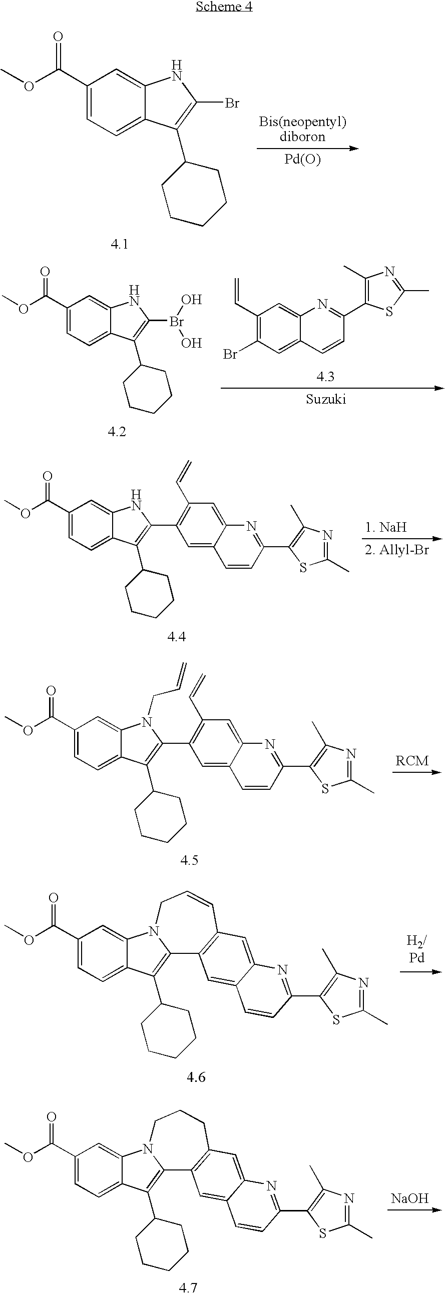

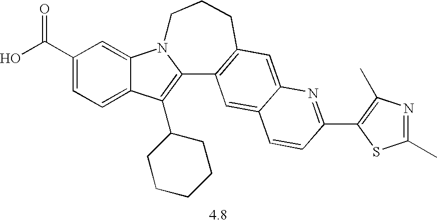

For illustrative purposes, Scheme 4 shows the synthesis of compounds where Z is COOH, Q is cyclohexyl, rings H and I are as depicted, and T is a substituted alkylene that forms a central seven member ring. Bromoindole derivative 4.1 is converted to the corresponding boronic acid 4.2 using bis(neopentyl)diboron and a palladium(0) catalyst. Suzuki coupling of the boronic acid 4.2 with allyl substituted aryl bromide 4.3 gives the biaryl product 4.4. Base catalyzed N-allylation of indole 4.4 with allyl bromide gives the di-N-allyl product 4.5. The ring closure metathesis (RCM) reaction of the di-N-allyl compound 4.5 gives the 7-membered carbacyclic product 4.6. This reaction is carried out using a metallocarbene complex as a catalyst such as by treatment of the diallyl compound 4.5 with about 10 mol % of Ruthenium benzylidene complex (Cy₃P)₂Ru(═CHPh)Cl₂ in a solvent such as methylene chloride gives the 7-membered carbacycle 4.6. The ruthenium catalyst, also commonly known as “Grubb's catalyst” or benzylidene-bis(tricyclohexylphosphine)dichlororuthenium, is commercially available from Sigma Aldrich. Several other commercially available metallocarbene complexes of metals such as Ruthenium and Molybdenum can also be used in this reaction. Hydrogenation of 4.6 gives 4.7 that is then saponified to 4.8.

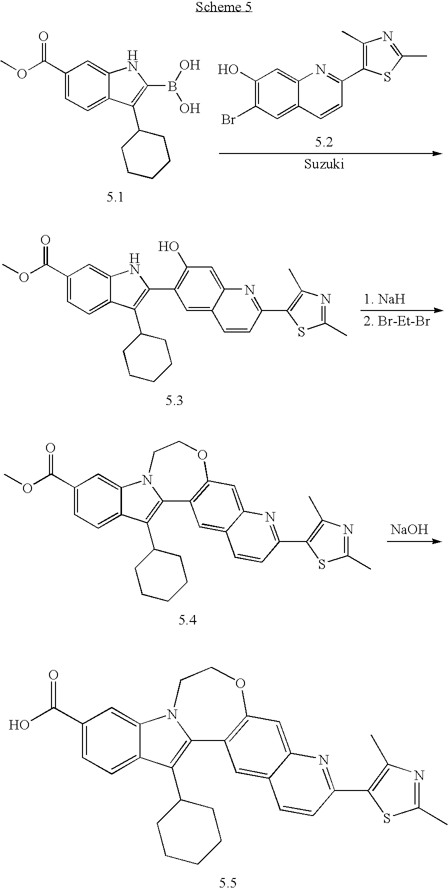

For illustrative purposes, Scheme 5 shows the synthesis of compounds where Z is COOH, Q is cyclohexyl, rings H and I are as depicted, and T is —CH₂CH₂O— that forms a central seven member ring. Boronic acid 5.1 is coupled with bromide 5.2 under Suzuki coupling conditions to give 5.3. Treatment of 5.3 with NaH and 1,2-dibromoethane gives 5.4 that is then saponified to yield 5.5.

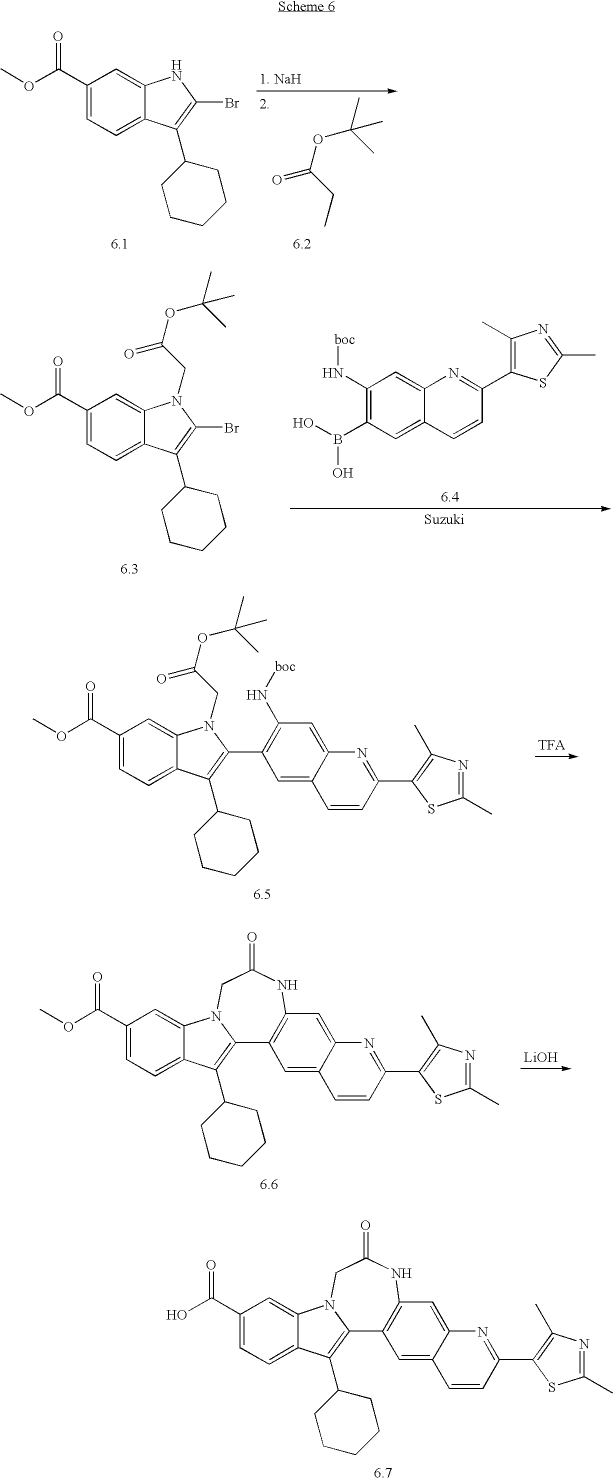

For illustrative purposes, Scheme 6 shows the synthesis of compounds where Z is COOH, Q is cyclohexyl, rings H and I are as depicted, and T is —CH₂C(O)NH— that forms a central seven member ring. Indole 6.1 is deprotonated and alkylated with 6.2 to give bromide 6.3 that is then coupled to boc (tert-butyloxycarbonyl) protected boronic acid 6.4 in the presence of a palladium(0) catalyst such as triphenylphosphine palladium(II) dichloride. Treatment of 6.5 with TFA (trifluoroacetic acid) effects ring closure to form lactam 6.6 that is saponifed under basic conditions such as with LiOH to give 6.7.

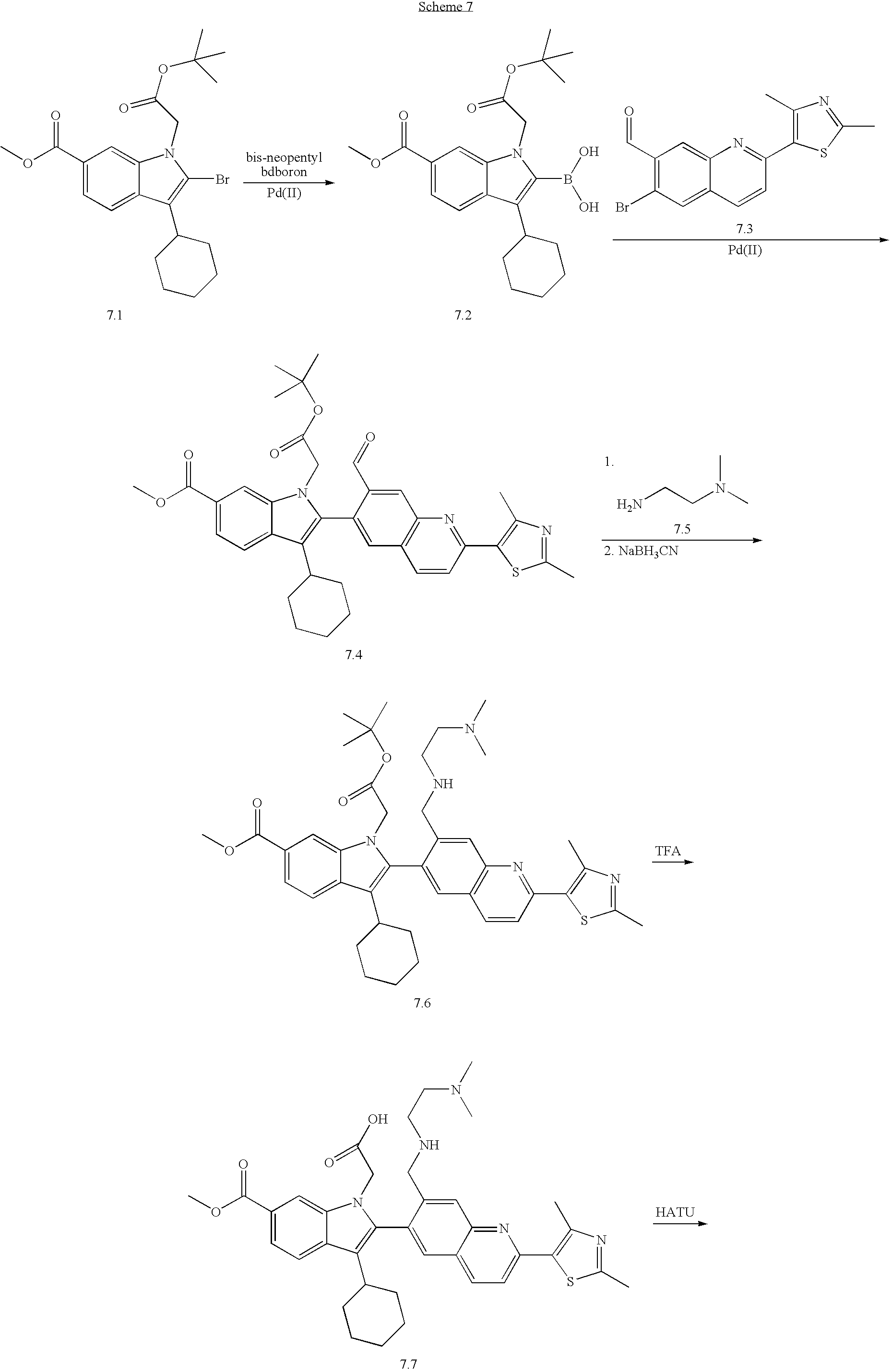

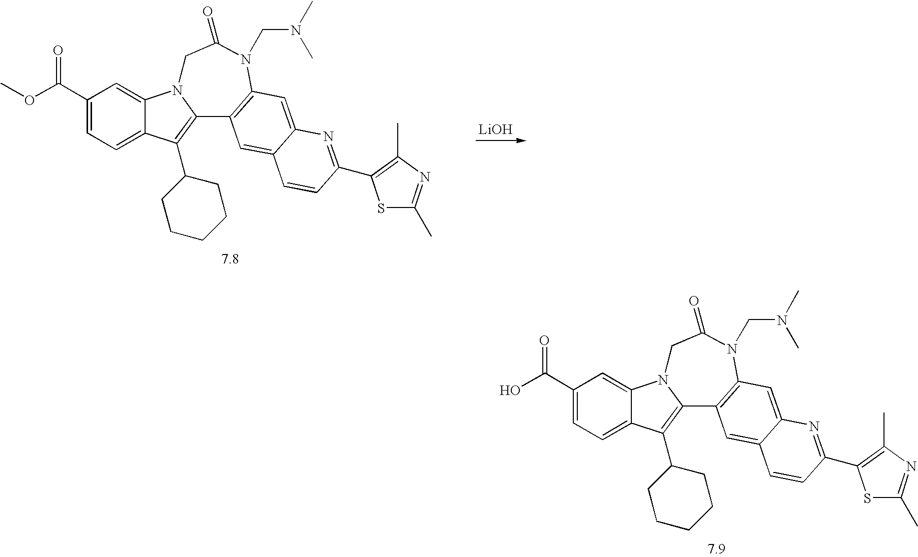

For illustrative purposes, Scheme 7 shows the synthesis of compounds where Z is COOH, Q is cyclohexyl, rings H and I are as depicted, and T forms a central eight member ring. Bromoindole derivative 7.1 is converted to the corresponding boronic acid 7.2 using bis(neopentyl)diboron and a palladium(0) catalyst. Suzuki coupling of the boronic acid 7.2 with 7.3 gives the biaryl product 7.4 followed by reductive amination with amine 7.5 and NaBH₃CN to give 7.6. Hydrolysis of the t-butyl ester group with trifluororoacetic acid gives acid 7.7 that is then cyclized to give amide 7.8. The ring closure can be affected with an amide coupling reagent such as HATU (N-[(dimethylamino)-1H-1,2,3-triazolo[4,5-b]pyridine-1-ylmethylene]-N-methylmethanaminium hexafluorophosphate N-oxide). Saponification of 7.8 with base such as with LiOH gives 7.9.

Various starting materials used in the Schemes above may be prepared from the corresponding 2-bromoindole derivatives which are known in the art and disclosed, for example, in International Patent Application Publication No. WO 03/010141 which is incorporated herein by reference in its entirety. Scheme 8 illustrates the conversion of 2-bromoindole derivatives to the corresponding indol-2-yl boronic acid.

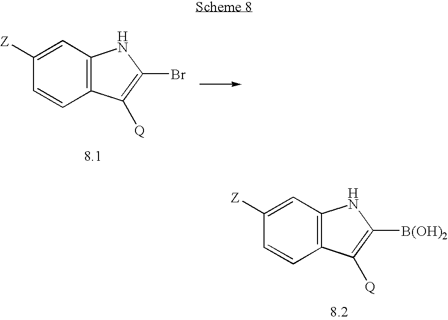

Specifically, compound 8.1 is converted to the 2-boronic acid derivative, compound 8.2, by contact with an excess of bis(neopentylglycolato)diboron in the presence of a catalytic amount of triphenylphosphine palladium(II) dichloride. The reaction is conducted in a suitable solvent, such as DMSO, in the presence of a suitable base such as potassium acetate under an inert atmosphere. Preferably, the reaction is conducted at a temperature of from about 60° C. to about 120° C. The reaction is continued until it is substantially complete which typically occurs within about 0.5 to 15 hours.

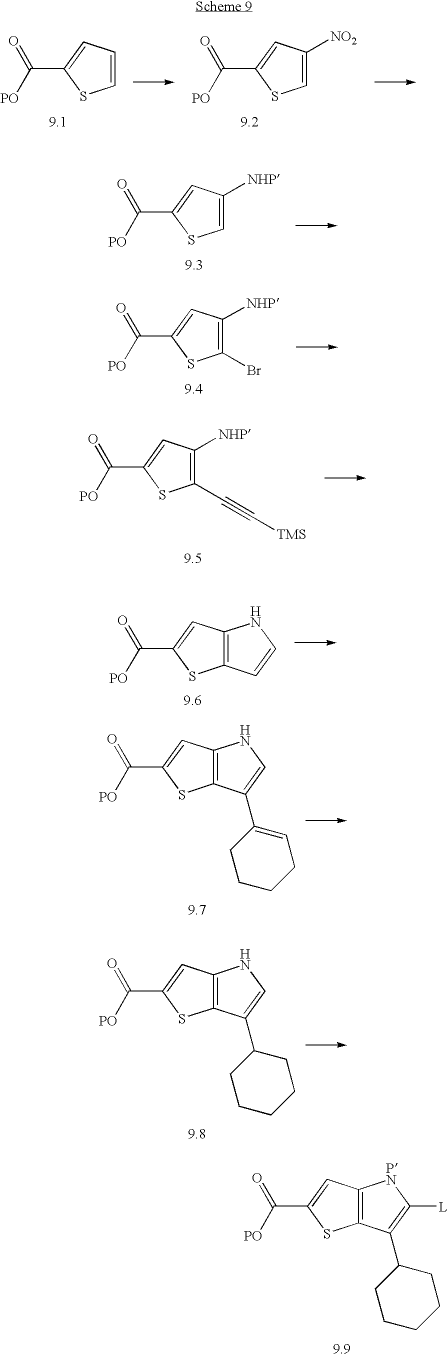

Other starting materials used in the Schemes above may be prepared as shown in Scheme 9 above where for illustrative purposes D is CH, E is S, Z is COOP, Q is cyclohexyl, P is a hydroxy protecting group such as alkyl, P′ is a nitrogen protecting group, and L is halogen. Thiophene 9.1 is treated with a mixture of nitric and sulfuric acid to form nitro compound 9.2. Reduction of the nitro group followed by protection of the resulting amine with a protecting group P′ such as t-butyloxycarbonyl affords compound 9.3. Thiophene 9.3 can be treated with a halogenating agent such as N-bromosuccinimide (NBS) to form bromide 9.4. Exposure of 9.4 with trimethylsilylacetylene, CuI, and PdCl₂(PPh₃)₂ gives acetylene 9.5 that is then treated with n-Bu₄NF and exposed to microwave radiation to form 9.6. Compound 9.6 is next reacted with cyclohexanone and sodium ethoxide in ethanol under refluxing conditions to form cyclohexene 9.7 that is then reduced to cyclohexane 9.8 with H₂ and Pd(OH)₂/C or with a reducing agent such as triethylsilane. Compound 9.8 can then be protected followed by treatment with a halogenating agent such as NBS to form the coupling partner 9.9 wherein the protecting group P′ may optionally be removed.

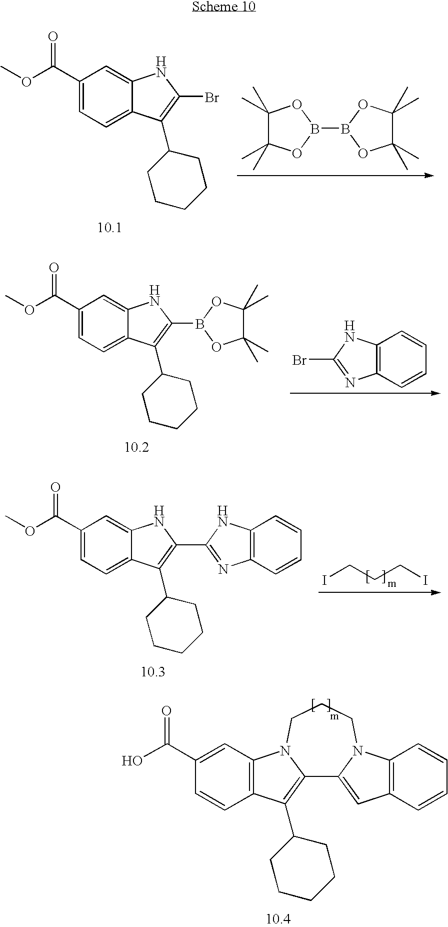

For illustrative purposes, Scheme 10 shows the synthesis of compounds where Z is COOH, Q is cyclohexyl, rings H and I form a benzimidazole group, and T is an alkylene chain where m is 0, 1, or 2. Bromide 10.1 synthesized as described in WO 2003010141 is reacted with bis(pinacolato)diboron and catalytic Pd(PPh₃)₂Cl₂ to give ester 10.2 that is then coupled with 2-bromobenzimidazole under Suzuki coupling conditions to give 10.3. Treatment of 10.3 with a base such as NaH and a dihaloalkane such as a diiodioalkane gives a cyclized intermediate that is then saponified to yield 10.4.

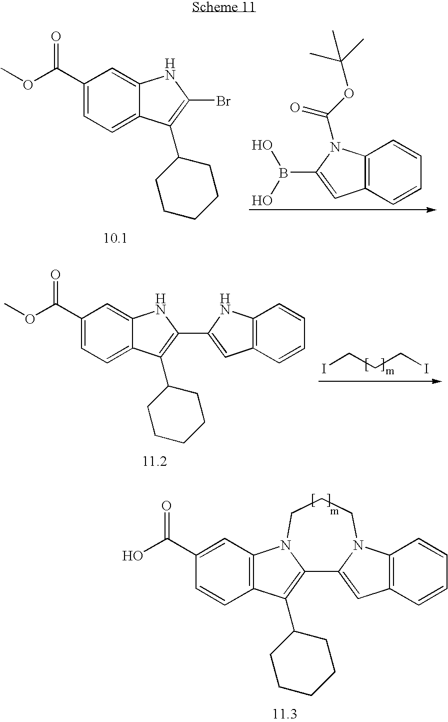

For illustrative purposes, Scheme 11 shows the synthesis of compounds where Z is COOH, Q is cyclohexyl, rings H and I form an indole group, and T is an alkylene chain where m is 0, 1, or 2. Bromide 10.1 is reacted with an indole boronic acid and catalytic Pd(PPh₃)₂Cl₂ under Suzuki coupling conditions to give 11.2. Treatment of 11.2 with a base such as NaH and a dihaloalkane such as a diiodioalkane gives a cyclized intermediate that is then saponified to yield 11.3.

Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-70 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J. USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiquimod, ribavirin, an inosine 5′monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

### EXAMPLES

In the examples below and the synthetic schemes above, the following abbreviations have the following meanings. If an abbreviation is not defined, it has its generally accepted meaning.

| | |
|---|---|
| μL = | microliters |
| μM = | micromolar |
| μg = | micrograms |
| NMR = | nuclear magnetic resonance |
| br = | broad |
| d = | doublet |
| δ = | chemical shift |
| dd = | doublet of doublets |
| DMEM = | Dulbeco's Modified Eagle's Medium |
| DMF = | N,N-dimethylformamide |
| DMSO = | dimethylsulfoxide |
| DTT = | dithiothreotol |
| EDTA = | ethylenediaminetetraacetic acid |
| ESI = | electrospray ionization |
| g = | gram |
| h or hr = | hours |
| HCV = | hepatitus C virus |
| HPLC = | high performance liquid chromatography |
| Hz = | hertz |
| IPTG = | isopropyl-β-D-thiogalactopyranoside |
| IU = | International Units |
| IC₅₀ = | inhibitory concentration at 50% inhibition |
| J = | coupling constant (given in Hz unless otherwise |
| | indicated) |
| m = | multiplet |
| M = | molar |
| M + H⁺= | parent mass spectrum peak plus H⁺ |
| mg = | milligram |
| mL = | milliliter |
| mM = | millimolar |
| mmol = | millimole |
| MS = | mass spectrum |
| nm = | nanometer |
| nM = | nanomolar |
| ng = | nanogram |
| NTA = | nitrilotriacetic acid |
| NTP = | nucleoside triphosphate |
| PCR = | Polymerase chain reaction |
| ppm = | parts per million |
| psi = | pounds per square inch |
| Rp-HPLC = | reversed phase high performance liquid |
| | chromatography |
| s = | singlet |
| t = | triplet |
| tetrakis or tetrakis | tetrakis(triphenylphosphine)palladium(0) |
| palladium = | |
| TFA = | trifluoroacetic acid |
| THF = | tetrahydrofuran |
| Tris = | Tris(hydroxymenthyl)aminomethane |
| UTP = | uridine triphosphate |

Set forth in the examples below are compounds and intermediates useful for making compounds of the present invention. An overview of the synthetic protocols employed to prepare these compounds is set forth above.

### Example 1

### Synthesis of Compound 225

### Step 1. 3-Cyclohexyl-6-methylcarboxylate-1H-indole-2-boronic acid pinacol ester (10.2)

A 250 mL reaction flask was charged with 3 g (8.9 mmol) 10.1, 6.8 g (26.8 mmol, 3 eq) bis(pinacolato)diboron, 2.6 g (26.5 mmol, 3 eq) potassium acetate and 312 mg (0.45 mmol, 0.05 eq) Pd(PPh₃)₂Cl₂. The mixture was then suspended with 60 mL dioxane, and subsequently degassed and purged with Argon (3×). The reaction mixture was then heated gently to 90° C. for 16.5 h. HPLC analysis confirmed complete consumption of 10.1. The reaction mixture was allowed to cool to room temperature and then concentrated. The crude residue was dissolved with EtOAc and purified twice by SiO₂ chromatography (10→20% EtOAc in hexane) to afford 1.76 mg (52%) of 10.2 as a white powder. MS: 384.2 (M+H⁺).

### Step 2. 2-(1H-Benzoimidazol-2-yl)-3-cyclohexyl-1H-indole-6-carboxylic acid methyl ester (10.3)

A reaction flask was charged with 100 mg (0.26 mmol) 10.2, 64 mg (0.33 mmol, 1.25 eq) 2-bromobenzimidazole and 15 mg (0.013 mmol, 0.05 eq) Pd(PPh₃)₄. To this was added 4 mL dioxane and 1 mL NaHCO₃ (sat., aq.). The reaction mixture was degassed and purged with Argon (2×), and then gently heated to 90° C. for 4 h. HPLC and LC-MS analysis confirmed complete consumption of 10.2. The reaction mixture was allowed to cool to room temperature and then concentrated. The desired methyl ester was precipitated by addition of cold H₂O and collected by centrifuge. 10.3 was dried under vacuum and used without further purification. MS: 374.1 (M+H⁺).

### Step 3. Synthesis of Compound 225

A reaction vessel was charged with 97 mg (0.26 mmol) 10.3 and dissolved with 10 mL DMF. NaH (31 mg, 0.78 mmol, 3 eq, 60% in mineral oil) was then added and the mixture was allowed to stir at room temperature for 15 min. 1,3-diiodopropane (37 μL, 0.33 mmol, 1.25 eq) was then added via syringe and the reaction mixture was allowed to continue stirring at room temperature. HPLC and LC-MS analysis confirmed complete consumption of 10.3 after 1 h. The reaction mixture was poured into a 50 mL flask and concentrated. The methyl ester was isolated by addition of cold H₂O to induce precipitation. The resulting solids were then collected by centrifuge and dissolved with 3 mL THF, 1 mL MeOH and 1 mL LiOH (1M, aq.). The reaction mixture was allowed to stir at 50° C. and monitored by HPLC analysis. When complete, the reaction mixture was neutralized with 0.5 mL HCl (2M, aq.) and concentrated. The crude residue was dissolved with DMF and acidified with TFA. The solution was then filtered and purified by reverse-phase HPLC to afford 19 mg (16%) compound 225 as a white powder. MS: 400.2 (M+H⁺); ¹H NMR (DMSO-d6): δ 8.32 (s, 1H), 8.02 (d, J=8.7, 1H), 7.90-7.86 (m, 2H), 7.71 (dd, J=8.7, 1.2, 1H), 7.48-7.37 (m, 2H), 4.41-4.39 (m, 4H), 3.49-3.45 (m, 1H), 2.12-1.41 (m, 12H).

### Example 2

### Synthesis of Compound 226

A reaction vessel was charged with 97 mg (0.26 mmol) 10.3 and dissolved with 10 mL DMF. NaH (31 mg, 0.78 mmol, 3 eq, 60% in mineral oil) was then added and the mixture was allowed to stir at room temperature for 15 min. 1,4-diiodobutane (43 μL, 0.33 mmol, 1.25 eq) was then added via syringe and the reaction mixture was allowed to continue stirring at room temperature. HPLC and LC-MS analysis confirmed complete consumption of 10.3 after 4 h during which time addition portions of both NaH and 1,4-diiodobutane were added. The reaction mixture was poured into a 50 mL flask and concentrated. The methyl ester was isolated by addition of cold H₂O to induce precipitation. The resulting solids were then collected by centrifuge and dissolved with 3 mL THF, 1 mL MeOH and 1 mL LiOH (1M, aq.). The reaction mixture was allowed to stir at 50° C. and monitored by HPLC analysis. When complete, the reaction mixture was neutralized with 0.5 mL HCl (2M, aq.) and concentrated. The crude residue was dissolved with DMF and acidified with TFA. The solution was then filtered and purified by reverse-phase HPLC to afford 20 mg (19%) compound 226 as a white powder. MS: 414.2 (M+H⁺); ¹H NMR (DMSO-d6): δ8.21 (s, 1H), 8.00 (d, J=8.6, 1H), 7.89 (d, J=7.1, 1H), 7.86 (d, J=8.0, 1H), 7.75 (d, J=8.6, 1H), 7.52-7.43 (m, 2H), 4.69-4.65 (m, 2H), 3.67-3.49 (m, 2H), 2.98-2.94 (m, 1H), 2.16-1.22 (m, 14H).

### Example 3

### Synthesis of Compound 227

### Step 1. 3-Cyclohexyl-1H,1′H-[2,2′]biindolyl-6-carboxylic acid methyl ester (11.2)

A microwave reaction vessel was charged with 100 mg (0.30 mmol) 10.1, 97 mg (0.37 mmol, 1.25 eq) 1-Boc-indole-2-boronic acid and 17 mg (0.015 mmol, 0.05 eq) Pd(PPh₃)₄. To this was added 4 mL dioxane and 1 mL K₃PO₄ (1M, aq.). The reaction vessel was sealed, and subsequently degassed and purged with Argon (1×). The reaction mixture was then heated by microwave to 120° C. for 10 min. HPLC and LC-MS analysis confirmed complete consumption of 10.1. The reaction mixture was allowed to cool to room temperature, transferred to a 50 mL flask and concentrated. The resulting residue was dissolved with 4 mL TFA and allowed to stir at room temperature. After 30 min, HPLC analysis showed complete deprotection. The reaction mixture was then concentrated. The desired methyl ester was precipitated by addition of cold H₂O and collected by centrifuge. Compound 11.2 was dried under vacuum and used without further purification. MS: 373.1 (M+H⁺)

### Step 2. Synthesis of compound 227

A reaction vessel was charged with 111 mg (0.30 mmol) 11.2 and dissolved with 10 mL DMF. NaH (36 mg, 0.89 mmol, 3 eq, 60% in mineral oil) was then added and the mixture was allowed to stir at room temperature for 15 min. 1,3-diiodopropane (43 μL, 0.37 mmol, 1.25 eq) was then added via syringe and the reaction mixture was allowed to continue stirring at room temperature. HPLC and LC-MS analysis confirmed complete consumption of 11.2 after 70 min. At that time, 1 mL NaOH (2M, aq.) was added. The mixture was heated with stirring to 80° C. and monitored by HPLC analysis. When complete, the reaction mixture was neutralized with 1 mL HCl (2M, aq.) and concentrated. The crude residue was dissolved with DMF and acidified with TFA. The solution was then filtered and purified by reverse-phase HPLC to afford 11 mg (9%) 227 as an off-white powder. MS: 399.1 (M+H⁺); ¹H NMR (DMSO-d6): δ12.7 (br s, 1H), 8.22 (d, J=1.2, 1H), 7.92 (d, J=8.7, 1H), 7.72-7.65 (m, 3H), 7.26 (td, J=8.1, 1.2, 1H), 7.15 (t, J=7.8, 1H), 6.76 (s, 1H), 4.28-4.23 (m, 4H), 3.14-2.93 (m, 1H), 2.43-1.28 (m, 12H).

### Example 4

### Synthesis of Compound 228

A reaction vessel was charged with 111 mg (0.30 mmol) 11.2 and dissolved with 10 mL DMF. NaH (36 mg, 0.89 mmol, 3 eq, 60% in mineral oil) was then added and the mixture was allowed to stir at room temperature for 15 min. 1,4-diiodobutane (49 μL, 0.37 mmol, 1.25 eq) was then added via syringe and the reaction mixture was allowed to continue stirring at 35° C. HPLC and LC-MS analysis confirmed complete consumption of 11.2 after 125 min. At that time, 1 mL NaOH (2M, aq.) was added. The mixture was heated with stirring to 80° C. and monitored by HPLC analysis. When complete, the reaction mixture was concentrated by speed-vacuum. The crude residue was dissolved with DMF and acidified with TFA. The solution was then filtered and purified by reverse-phase HPLC to afford 31 mg (25%) compound 228 as an off-white powder. MS: 413.2 (M+H⁺); ¹H NMR (DMSO-d6): δ12.7 (br s, 1H), 8.13 (d, J=0.9, 1H), 7.94 (d, J=8.3, 1H), 7.72 (d, J=8.3, 2H), 7.60 (d, J=8.0, 1H), 7.28 (td, J=8.3, 1.2, 1H), 7.16 (t, J=7.7, 1H), 6.70 (s, 1H), 4.57-4.53 (m, 2H), 3.41-3.31 (m, 2H), 2.93-2.90 (m, 1H), 2.13-1.25 (m, 14H).

### Example 5

### Synthesis of Compound 229

A reaction vessel was charged with 110 mg (0.30 mmol) 11.2 and dissolved with 10 mL DMF. NaH (35 mg, 0.89 mmol, 3 eq, 60% in mineral oil) was then added and the mixture was allowed to stir at room temperature for 15 min. 1,4-diiodopentane (55 μL, 0.37 mmol, 1.25 eq) was then added via syringe and the reaction mixture was allowed to continue stirring at room temperature. HPLC and LC-MS analysis confirmed complete consumption of 11.2 after 70 min. At that time, 1 mL NaOH (2M, aq.) was added. The mixture was heated with stirring to 80° C. and monitored by HPLC analysis. When complete, the reaction mixture was concentrated by rotovap. The crude residue was dissolved with DMF and acidified with TFA. The solution was then filtered and purified by reverse-phase HPLC to afford 13 mg (10%) compound 229 as an off-white powder. MS: 427.2 (M+H⁺); ¹H NMR (DMSO-d6): δ12.7 (br s, 1H), 8.09 (d, J=0.9, 1H), 7.90 (d, J=8.4, 1H), 7.74-7.70 (m, 2H), 7.57 (d, J=8.1, 1H), 7.27 (td, J=8.4, 1.4, 1H), 7.16 (t, J=6.9, 1H), 6.69 (s, 1H), 4.42-4.36 (m, 2H), 3.47-2.93 (m, 2H), 2.77-2.68 (m, 1H), 1.88-1.23 (m, 16H).

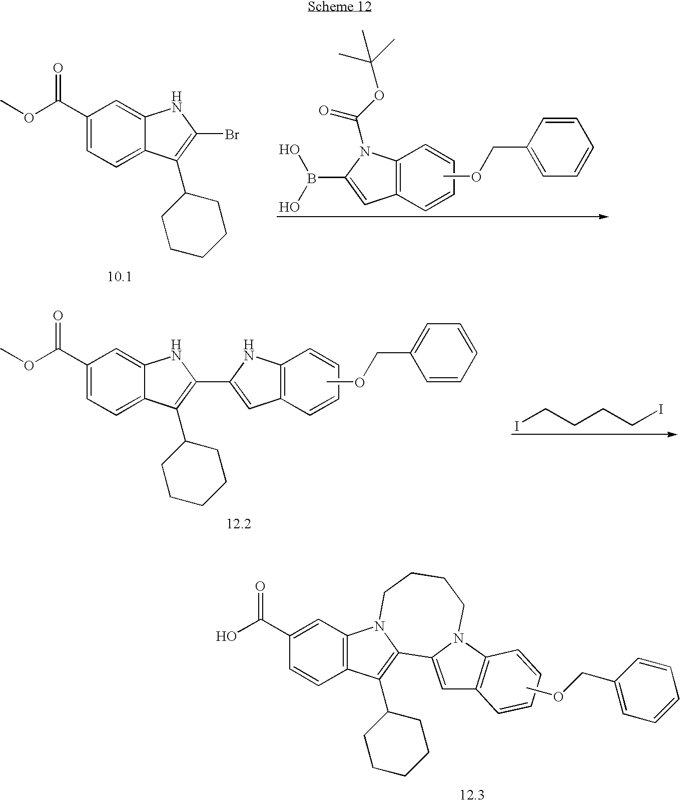

### Example 6

### Synthesis of Compound 230

Step 1. 4′-Benzyloxy-3-cyclohexyl-1H,1′H-[2,2′]biindolyl-6-carboxylic acid methyl ester (12.2a)

A microwave reaction vessel was charged with 150 mg (0.45 mmol) 10.1, 213 mg (0.58 mmol, 1.3 eq) 1-Boc-4-benzyloxyindole-2-boronic acid and 26 mg (0.022 mmol, 0.05 eq) Pd(PPh₃)₄. To this was added 6 mL dioxane and 1.5 mL K₃PO₄ (1M, aq.). The reaction vessel was sealed, and subsequently degassed and purged with Argon (1×). The reaction mixture was then heated by microwave to 80° C. for 10 min. HPLC and LC-MS analysis confirmed complete consumption of 10.1. The reaction mixture was allowed to cool to room temperature and concentrated by speed-vacuum. The resulting residue was dissolved with 2 mL TFA and allowed to stir at room temperature until complete by HPLC analysis. The reaction mixture was then concentrated and dried under vacuum. Compound 12.2a was used without further purification. MS: 479.1 (M+H⁺).

### Step 2. Synthesis of Compound 230

A reaction vessel was charged with 107 mg (0.22 mmol) 12.2a and dissolved with 10 mL DMF. NaH (27 mg, 0.67 mmol, 3 eq, 60% in mineral oil) was then added and the mixture was allowed to stir at room temperature for 20 min. 1,4-diiodobutane (37 μL, 0.28 mmol, 1.25 eq) was then added via syringe and the reaction mixture was allowed to continue stirring at room temperature. HPLC and LC-MS analysis confirmed complete consumption of 12.2a after 60 min. The reaction mixture was then concentrated and the methyl ester was precipitated with H₂O. The solids were collected by centrifuge and dissolved with 3 mL THF, 1 mL MeOH and 1 mL LiOH (1M, aq.). The mixture was then heated with stirring to 80° C. and monitored by HPLC analysis. When complete, the reaction mixture was neutralized with 0.5 mL HCl (2M, aq.) and concentrated. The crude residue was dissolved with DMF and acidified with TFA. The solution was then filtered and purified by reverse-phase HPLC to afford 12 mg (10%) compound 230 as an off-white powder. MS: 519.2 (M+H⁺); ¹H NMR (DMSO-d6): δ12.7 (br s, 1H), 8.12 (s, 1H), 7.93 (d, J=8.6, 1H), 7.71 (dd, J=8.4, 1.4, 1H), 7.57-7.54 (m, 2H), 7.46-7.33 (m, 3H), 7.22-7.15 (m, 2H), 6.76 (dd, J=6.6, 2.0, 1H), 6.68 (s, 1H), 5.33 (s, 2H), 4.59-4.46 (m, 2H), 3.43-3.32 (m, 2H), 2.89-2.77 (m, 1H), 2.13-1.27 (m, 14H).

### Example 7

### Synthesis of Compound 231

### Step 1. 5′-Benzyloxy-3-cyclohexyl-1H,1′H-[2,2′]biindolyl-6-carboxylic acid methyl ester (12.2b)

A microwave reaction vessel was charged with 100 mg (0.30 mmol) 10.1, 120 mg (0.33 mmol, 1.3 eq) 1-Boc-5-benzyloxyindole-2-boronic acid and 17 mg (0.015 mmol, 0.05 eq) Pd(PPh₃)₄. To this was added 4 mL dioxane and 0.8 mL K₃PO₄ (1M, aq.). The reaction vessel was sealed, and subsequently degassed and purged with Argon (1×). The reaction mixture was then heated by microwave to 70° C. for 15 min. HPLC and LC-MS analysis confirmed complete consumption of 10.1. The reaction vessel was then resealed and heated to by microwave to 160° C. to cleave the Boc-group. The reaction mixture was then concentrated and the desired methyl ester was precipitated with H₂O. The solids were collected by centrifuge and dried under vacuum. 12.2b was used without further purification. MS: 479.1 (M+H⁺).

### Step 2. Synthesis of Compound 231

A reaction vessel was charged with 142 mg (0.30 mmol) 12.2b and dissolved with 10 mL DMF. NaH (36 mg, 0.89 mmol, 3 eq, 60% in mineral oil) was then added and the mixture was allowed to stir at room temperature for 20 min. 1,4-diiodobutane (49 μL, 0.37 mmol, 1.25 eq) was then added via syringe and the reaction mixture was allowed to continue stirring at room temperature. HPLC and LC-MS analysis confirmed complete consumption of 12.2b after 80 min. The reaction mixture was then concentrated and the methyl ester was precipitated with H₂O. The solids were collected by centrifuge and dissolved with 3 mL THF, 1 mL MeOH and 1 mL LiOH (1M, aq.). The mixture was then heated with stirring to 80° C. and monitored by HPLC analysis. When complete, the reaction mixture was neutralized with 0.5 mL HCl (2M, aq.) and concentrated. The crude residue was dissolved with DMF and acidified with TFA. The solution was then filtered and purified by reverse-phase HPLC to afford 20 mg (17%) compound 231 as a yellow powder. MS: 519.2 (M+H⁺); ¹H NMR (DMSO-d6): δ12.7 (br s, 1H), 8.12 (s, 1H), 7.93 (d, J=8.3, 1H), 7.71 (d, J=8.3, 1H), 7.53-7.30 (m, 7H), 7.01 (dd, J=8.9, 2.3, 1H), 6.59 (s, 1H), 5.19 (s, 2H), 4.52 (td, J=14.9, 6.3, 2H), 3.5-3.3 (m, 2H), 2.88 (m, 1H), 2.12-1.27 (m, 14H).

### Example 8

### Synthesis of Compound 232

### Step 1. 6′-Benzyloxy-3-cyclohexyl-1H,1′H-[2,2′]biindolyl-6-carboxylic acid methyl ester (12.2c)

A microwave reaction vessel was charged with 150 mg (0.45 mmol) 10.1, 213 mg (0.58 mmol, 1.3 eq) 1-Boc-6-benzyloxyindole-2-boronic acid and 26 mg (0.022 mmol, 0.05 eq) Pd(PPh₃)₄. To this was added 6 mL dioxane and 1.5 mL K₃PO₄ (1M, aq.). The reaction vessel was sealed, and subsequently degassed and purged with Argon (1×). The reaction mixture was then heated by microwave to 80° C. for 10 min. HPLC and LC-MS analysis confirmed complete consumption of 10.1. The reaction mixture was allowed to cool to room temperature and concentrated by speed-vacuum. The resulting residue was dissolved with 2 mL TFA and allowed to stir at room temperature until complete by HPLC analysis. The reaction mixture was then concentrated and the desired methyl ester was precipitated with H₂O. The solids were collected by centrifuge and washed with additional H₂O (2×). Compound 12.2c was dried under vacuum and used without further purification. MS: 479.1 (M+H⁺).

### Step 2. Synthesis of Compound 232

A reaction vessel was charged with 107 mg (0.22 mmol) 12.2c and dissolved with 10 mL DMF. NaH (27 mg, 0.67 mmol, 3 eq, 60% in mineral oil) was then added and the mixture was allowed to stir at room temperature for 20 min. 1,4-diiodobutane (37 μL, 0.28 mmol, 1.25 eq) was then added via syringe and the reaction mixture was allowed to continue stirring at room temperature until HPLC and LC-MS analysis confirmed complete consumption of 12.2c. The reaction mixture was then concentrated and the methyl ester was precipitated with H₂O. The solids were collected by centrifuge and dissolved with 3 mL THF, 1 mL MeOH and 1 mL LiOH (1M, aq.). The mixture was then heated with stirring to 80° C. and monitored by HPLC analysis. When complete, the reaction mixture was neutralized with 0.5 mL HCl (2M, aq.) and concentrated. The crude residue was dissolved with DMF and acidified with TFA. The solution was then filtered and purified by reverse-phase HPLC to afford 10 mg (9%) compound 232 as an off-white powder. ¹H NMR (DMSO-d6): δ 12.7 (br s, 1H), 8.11 (d, J=0.9, 1H), 7.92 (d, J=8.6, 1H), 7.70 (dd, J=8.3, 1.4, 1H), 7.60-7.37 (m, 6H), 7.26 (d, J=2.0, 1H), 6.89 (dd, J=8.6, 2.0, 1H), 6.61 (s, 1H), 5.23 (s, 2H), 4.58-4.44 (m, 2H), 3.3-3.6 (m, 2H), 2.89 (m, 1H), 2.13-1.33 (m, 14H).

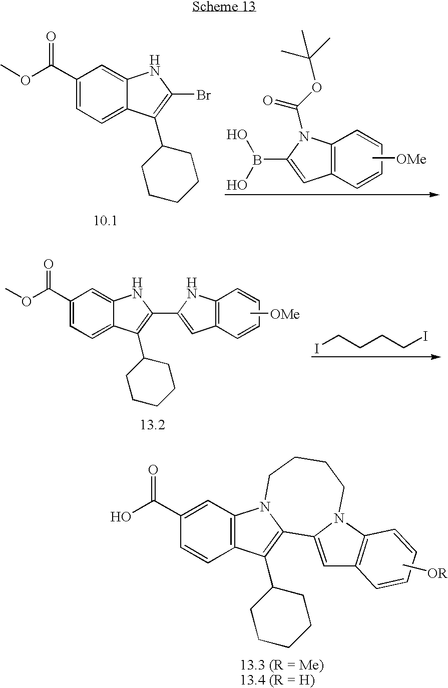

### Example 9

### Synthesis of Compound 233

### Step 1. 3-Cyclohexyl-5′-methoxy-1H,1′H-[2,2′]biindolyl-6-carboxylic acid methyl ester (13.2a)

A microwave reaction vessel was charged with 250 mg (0.74 mmol) 10.1, 646 mg (2.22 mmol, 3 eq) 1-Boc-5-methoxyindole-2-boronic acid and 43 mg (0.04 mmol, 0.05 eq) Pd(PPh₃)₄. To this was added 10 mL dioxane and 2.5 mL K₃PO₄ (1M, aq.). The reaction vessel was sealed, and subsequently degassed and purged with Argon (1×). The reaction mixture was then heated by microwave to 60° C. for 40 min. HPLC and LC-MS analysis confirmed complete consumption of 10.1. The reaction vessel was then resealed and heated to by microwave to 160° C. for 10 min to cleave the Boc-group. The reaction mixture was then concentrated and the desired methyl ester was precipitated with H₂O. The solids were collected by centrifuge and dried under vacuum. Compound 13.2a was used without further purification. MS: 403.2 (M+H⁺).

### Step 2. Synthesis of Compound 233

Step 2a: A reaction flask was charged with 298 mg (0.74 mmol) 13.2a and dissolved with 30 mL DMF. NaH (89 mg, 2.22 mmol, 3 eq, 60% in mineral oil) was then added and the mixture was allowed to stir at room temperature for 15 min. 1,4-diiodobutane (122 μL, 0.925 mmol, 1.25 eq) was then added via syringe and the reaction mixture was allowed to continue stirring at room temperature. HPLC and LC-MS analysis confirmed complete consumption of 13.2a after 2.5 h, during which time an additional 0.5 eq of both NaH and 1,4-diiodobutane was added. The reaction mixture was then concentrated and the methyl ester was precipitated with H₂O. The solids were collected by centrifuge and divided into 2 portions. Step 2b: One portion from step 2a was dissolved with 3 mL THF, 1 mL MeOH and 1 mL LiOH (1M, aq.). The mixture was then heated with stirring to 80° C. and monitored by HPLC analysis. When complete, the reaction mixture was neutralized with 0.5 mL HCl (2M, aq.) and concentrated by speed-vacuum. The crude residue was dissolved with DMF and acidified with TFA. The solution was then filtered and purified by reverse-phase HPLC to afford 24 mg (15%) compound 233 as a yellow powder. MS: 443.1 (M+H⁺); ¹H NMR (DMSO-d6): δ12.7 (br s, 1H), 8.12 (s, 1H), 7.93 (d, J=8.4, 1H), 7.71 (d, J=8.1, 1H), 7.50 (d, J=8.7, 1H), 7.22 (d, J=2.3, 1H), 6.92 (dd, J=8.7, 2.3, 1H), 6.60 (s, 1H), 4.59-4.46 (m, 2H), 3.84 (s, 3H), 3.34-3.29 (m, 2H), 2.93-2.77 (m, 1H), 2.16-1.27 (m, 14H).

### Example 10

### Synthesis of Compound 234

Following Step 2a from the preparation of compound 233, the second portion from step 2a was dissolved with 5 mL CH₂Cl₂ and 1.8 mL BBr3 (1M, CH₂Cl₂) was carefully added via syringe. The mixture was then allowed to stir at room temperature overnight. The reaction mixture was then poured into 2 mL HCl (2M, aq) and concentrated. The residue was then diluted with EtOAc and extracted twice with NaOH (2M, aq). The combined aqueous phase was acidified with HCl (conc., aq) and extracted with EtOAc. The organic phase was then washed with brine, dried over Na₂SO₄, filtered and concentrated. The crude residue was dissolved with DMF and acidified with TFA. The solution was then filtered and purified by reverse-phase HPLC to afford 8 mg (5%) compound 234 as an off-white powder. MS: 429.1 (M+H⁺); ¹H NMR (DMSO-d6): δ12.68 (br s, 1H), 8.88 (br s, 1H), 8.10 (s, 1H), 7.91 (d, J=8.4, 1H), 7.69 (dd, J=8.4, 1.5, 1H), 7.38 (d, J=8.7, 1H), 7.00 (d, J=2.0, 1H), 6.78 (dd, J=8.7, 2.0, 1H), 6.49 (s, 1H), 4.56-4.39 (m, 2H), 3.41-3.29 (m, 2H), 2.92-2.76 (m, 1H), 2.14-1.10 (m, 14H).

### Example 11

### Synthesis of Compound 235

### Step 1. 3-Cyclohexyl-6′-methoxy-1H,1′H-[2,2′]biindolyl-6-carboxylic acid methyl ester (13.2b)

A microwave reaction vessel was charged with 250 mg (0.74 mmol) 10.1, 646 mg (2.22 mmol, 3 eq) 1-Boc-6-methoxyindole-2-boronic acid and 43 mg (0.04 mmol, 0.05 eq) Pd(PPh₃)₄. To this was added 10 mL dioxane and 2.5 mL K₃PO₄ (1M, aq.). The reaction vessel was sealed, and subsequently degassed and purged with Argon (1×). The reaction mixture was then heated by microwave to 60° C. for 40 min. HPLC and LC-MS analysis confirmed complete consumption of 10.1. The reaction vessel was then resealed and heated to by microwave to 160° C. for 10 min to cleave the Boc-group. The reaction mixture was then concentrated and the desired methyl ester was precipitated with H₂O. The solids were collected by centrifuge and dried under vacuum. Compound 13.2b was used without further purification. MS: 403.2 (M+H⁺).

### Step 2. Synthesis of Compound 235

Step 2a: A reaction flask was charged with 298 mg (0.74 mmol) 13.2b and dissolved with 30 mL DMF. NaH (89 mg, 2.22 mmol, 3 eq, 60% in mineral oil) was then added and the mixture was allowed to stir at room temperature for 15 min. 1,4-diiodobutane (122 μL, 0.925 mmol, 1.25 eq) was then added via syringe and the reaction mixture was allowed to continue stirring at room temperature. HPLC and LC-MS analysis confirmed complete consumption of 13.2b after 2.5 h, during which time an additional 0.5 eq of both NaH and 1,4-diiodobutane was added. The reaction mixture was then concentrated and the methyl ester was precipitated with H₂O. The solids were collected by centrifuge and divided into 2 portions. Step 2b: One portion of step 2a was dissolved with 3 mL THF, 1 mL MeOH and 1 mL LiOH (1M, aq.). The mixture was then heated with stirring to 80° C. and monitored by HPLC analysis. When complete, the reaction mixture was neutralized with 0.5 mL HCl (2M, aq.) and concentrated by speed-vacuum. The crude residue was dissolved with DMF and acidified with TFA. The solution was then filtered and purified by reverse-phase HPLC to afford 28 mg (17%) compound 235 as an off-white powder. MS: 443.1 (M+H⁺); ¹H NMR (DMSO-d6): δ12.69 (br s, 1H), 8.11 (s, 1H), 7.92 (d, J=8.7, 1H), 7.70 (d, J=8.4, 1H), 7.58 (d, J=8.4, 1H), 7.13 (s, 1H), 6.81 (dd, J=8.4, 2.0, 1H), 6.61 (s, 1H), 4.52 (td, J=14.5, 5.5, 2H), 3.88 (s, 3H), 3.40-3.33 (m, 2H), 2.93-2.77 (m, 1H), 2.17-1.21 (m, 14H).

### Example 12

### Synthesis of Compound 236

Following Step 2a from the preparation of compound 235, the second portion from step 2a was dissolved with 5 mL CH₂Cl₂ and 1.8 mL BBr3 (1M, CH₂Cl₂) was carefully added via syringe. The mixture was then allowed to stir at room temperature overnight. The reaction mixture was then poured into 2 mL HCl (2M, aq) and concentrated. The residue was then diluted with EtOAc and extracted twice with NaOH (2M, aq). The combined aqueous phase was acidified with HCl (conc., aq) and extracted with EtOAc. The organic phase was then washed with brine, dried over Na₂SO₄, filtered and concentrated. The crude residue was dissolved with DMF and acidified with TFA. The solution was then filtered and purified by reverse-phase HPLC to afford 8 mg (5%) compound 236 as an off-white powder. MS: 429.1 (M+H⁺); ¹H NMR (DMSO-d6): δ12.67 (br s, 1H), 9.24 (br s, 1H), 8.10 (s, 1H), 7.90 (d, J=8.6, 1H), 7.69 (dd, J=8.4, 1.2, 1H), 7.48 (d, J=8.4, 1H), 6.84 (s, 1H), 6.69 (dd, J=8.4, 2.0, 1H), 6.54 (s, 1H), 4.53-4.30 (m, 4H), 2.76 (m, 1H), 2.15-1.26 (m, 14H).

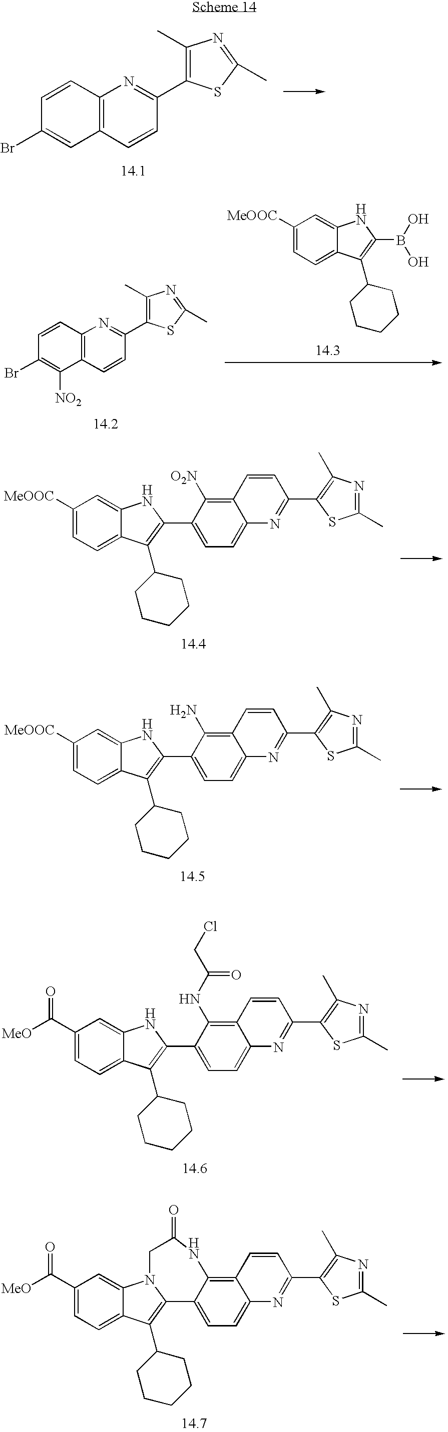

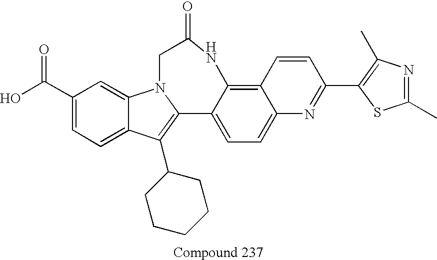

### Example 13

### Synthesis of Compound 237

### Step 1. 6-Bromo-5-nitro-2-(2,4-dimethyl-thiazol-5-yl)-quinoline (14.2)

6-Bromo-2-(2,4-dimethyl-thiazol-5-yl)-quinoline 14.1 (2.5 g, 7.83 mmol) was dissolved in concentrated H₂SO₄ (50 mL) and 90% nitric acid (2 mL) was added dropwise at 0° C. After addition, the mixture was stirred at 0° C. for 10 min and at room temperature for 1 h. The mixture was poured into ice-water (300 mL) and extracted with CH₂Cl₂-MeOH (8:1) (100 mL×5). The combined organic phase was washed with saturated NaHCO₃ (100 mL×2), water (50 mL×2) and dried over anhydrous Na₂SO₄. After evaporation of solvent, 14.2 was obtained (2.33 g, 82%). MS (M+H⁺): 363.9, 364.9, 365.9. ¹H NMR (CDCl₃) δ 8.05 (1H, d, J=7.7 Hz), 8.02 (1H, d, J=7.5 Hz), 7.87 (1H, d, J=9.0 Hz), 7.80 (1H, d, J=8.7 Hz), 2.78 (3H, s), 2.74 (3H, s). The product was used in the next step reaction without further purification.

### Step 2. 3-Cyclohexyl-2-[2-(2,4-dimethyl-thiazol-5-yl)-5-nitro-quinolin-6-yl]-1H-indole-6-carboxylic acid methyl ester (14.4)

A mixture of compound 14.2 (0.25 g, 0.685 mmol), 3-cyclohexyl-2-(5,5-dimethyl-[1,3,2]dioxaborinan-2-yl)-1H-indole-6-carboxylic acid methyl ester 14.3 (0.253 g, 0.685 mmol) and Pd(PPh₃)₄ (63 mg, 0.0548 mmol) in MeOH in the presence of saturated aqueous NaHCO₃ (2.25 ml) was stirred at 150° C. under Argon and under microwave irradiation for 10 min. After evaporation of solvent, the residue was purified by chromatography on silica gel eluting with CH₂Cl₂-MeOH (28:1) to yield 14.4 (0.28 g, 76%). MS (M+H⁺): 541.2.

### Step 3. 2-[5-Amino-2-(2,4-dimethyl-thiazol-5-yl)-quinolin-6-yl]-3-cyclohexyl-1H-indole-6-carboxylic acid methyl ester (14.5)

Compound 14.4 (0.28 g) was dissolved in MeOH-EtOAc (1:2) (15 mL) and shaken over 10% Pd/C (0.1 g) under 40 psi of H₂ at room temperate for 2 h. After filtration through celite, the filtrate was evaporated to give 14.5 in a quantitative yield. MS (M+H⁺): 511.2.

### Step 4. 2-[5-(2-Chloro-acetylamino)-2-(2,4-dimethyl-thiazol-5-yl)-quinolin-6-yl]-3-cyclohexyl-1H-indole-6-carboxylic acid methyl ester (14.6)

To a mixture of 14.5 (0.264 g, 0.517 mmol) and AcONa (46.7 mg, 0.568 mmol) in anhydrous THF (15 mL) in the presence of AcOH (32.6 μL, 0.568 mmol) was added chloroacetyl chloride (45.3 μL, 0.568 mmol). The reaction mixture was stirred at room temperate for 3 h. Aqueous saturated NaHCO₃ (50 mL) was added and the mixture was extrated with EtOAc (80 mL×2). The combined organic phase was washed with brine (50 mL) and water (50 mL), and dried over anhydrous Na₂SO₄. After evaporation of solvent, compound 14.6 was obtained (0.293 g, 97%). MS (M+H⁺): 587.2. This compound was directly used in the next step reaction without further purification.

### Step 5. Synthesis of Compound 14.7

To a solution of 14.6 (0.293 g, 0.499 mmol) in anhydrous DMF (5 mL) was added NaH (26.3 mg, 1.1 mmol) at 0° C. under Argon. The mixture was stirred at 0° C. for 20 min and at room temperature for 3 h. The mixture was then poured into ice-water (50 mL). Precipitates were collected by centrifuge and washed with water and dried to afford compound 14.7 (0.275 g, 99%). MS (M+H⁺): 551.2.

### Step 6. Synthesis of Compound 237

Compound 14.7 (48 mg) was dissolved in MeOH-THF (1:1) (1.5 mL) and 2 M LiOH (0.5 mL) was added. The mixture was stirred at room temperature for 2.5 h. The mixture was neutralized with 2 N HCl to pH 7. After evaporation of solvent, the residue was purified by reverse phase HPLC to give compound 237 (26 mg, 56%). MS (M+H⁺): 537.2. ¹H NMR (DMSO-d6) δ 10.69 (1H, s), 8.70 (1H, d, J=9.3 Hz), 8.30 (1H, s), 8.00-7.95 (3H, m), 7.84 (1H, d, J=9.0 Hz), 7.69 (1H, d, J=8.1 Hz), 5.15 (1H, d, J=14.7 Hz), 4.61 (1H, d, J=14.4 Hz), 2.95 (1H, m), 2.74 (3H, s), 2.68 (3H, s), 2.11-1.16 (10H, m).

### BIOLOGICAL EXAMPLES

### Biological Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al In vitro assays have been reported in Ferrari et al. Jnl. of Vir., 73:1649-1654, 1999; Ishii et al, Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application Ser. No. 60/004,383, filed on September 1995, described an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1 (Supp 4) 18-24.

Screens that measure reductions in kinase activity from HCV drugs were disclosed in U.S. Pat. No. 6,030,785, to Katze et al, U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al, U.S. Pat. No. 5,739,002 to De Francesco et al, and U.S. Pat. No. 5,597,691 to Houghton et al.

### Biological Example 2

### Replicon Assay

A cell line, ET (Huh-lucubineo-ET) was used for screening of compounds for inhibiting HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). Reagents are all available through Life Technologies (Bethesda, Md.). The cells were plated at 0.5−1.0×10⁴ cells/well in the 96 well plates and incubated for 24 hrs before adding test compound. The compounds were added to the cells to achieve a final concentration of 0.1 nM to 50 μM and a final DMSO (dimethylsulfoxide) concentration of 0.5%. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data is plotted relative to no compound control. Under the same condition, cytotoxicity of the compounds were determined using cell proliferation reagent, WST-1 (Roche, Germany). The compounds showing antiviral activities, but no significant cytotoxicities were chosen to determine EC₅₀ and TC₅₀ the effective concentration and toxic concentration at which 50% of the maximum inhibition is observed. For these determinations, a 10 point, 2-fold serial dilution for each compound was used, which spans a concentration range of 1000 fold. EC₅₀ and similarly TC₅₀ values were calculated by fitting % inhibition at each concentration to the following equation:
% inhibition=100%/[(EC₅₀/[I])_{b}+1]
where b is Hill's coefficient.

In some aspects, the compounds of Formula (A) will have an EC₅₀ of equal to or less than 50 μM when tested according to the assay of Example 2. In other aspects the EC₅₀ is equal to or less than 10 μM. Is still other aspects the EC₅₀ is equal to or less than 5 μM.

When tested at 25 μM, compounds 225-237 where found respectively to have the following % inhibition values in the Table below.

| Compound # | % inhibition |
|---|---|
| 225 | 7 |
| 226 | 23 |
| 227 | 94 |
| 228 | 97 |
| 229 | 86 |
| 230 | 75 |
| 231 | 96 |
| 232 | 91 |
| 233 | 96 |
| 234 | 99 |
| 235 | 99 |
| 236 | 100 |
| 237 | 100 |

### Biological Example 3

### Cloning and Expression of Recombinant HCV-NS5b

The coding sequence of NS5b protein was cloned by PCR from pFKI₃₈₉luc/NS3-3′/ET as described by Lohmann, V., et al. (1999) Science 285, 110-113 using the primers shown on page 266 of WO 2005/012288

The cloned fragment is missing the C terminus 21 amino acid residues. The cloned fragment was inserted into an IPTG-inducible (isopropyl-β-D-thiogalactopyranoside) expression plasmid that provides an epitope tag (His)6 at the carboxy terminus of the protein.

The recombinant enzyme was expressed in XL-1 cells and after induction of expression, the protein is purified using affinity chromatography on a nickel-NTA (nitrilotriacetic acid) column. Storage condition is 10 mM Tris-HCl pH 7.5, 50 mM NaCl, 0.1 mM EDTA (ethylenediaminetetraacetic acid), 1 mM DTT (dithiothreotol), 20% glycerol at −20° C.

### Biological Example 4

### HCV-NS5b Enzyme Assay

The polymerase activity was assayed by measuring incorporation of radiolabeled UTP into a RNA product using a biotinylated, heteropolymeric template, which includes a portion of the HCV genome. Typically, the assay mixture (50 μL) contains 10 mM Tris-HCl (pH 7.5), 5 mM MgCl₂, 0.2 mM EDTA, 10 mM KCl, 1 unit/μL RNAsin, 1 mM DTT, 10 μM each of NTP (nucleoside triphosphate), including [³H]-UTP (uridine triphosphate), and 10 ng/μL heteropolymeric template. Test compounds were initially dissolved in 100% DMSO and further diluted in aqueous buffer containing 5% DMSO. Typically, compounds were tested at concentrations between 1 nM and 100 μM. Reactions were started with addition of enzyme and allowed to continue at 37° C. for 2 hours. Reactions were quenched with 8 μL of 100 mM EDTA and reaction mixtures (30 μL) were transferred to streptavidin-coated scintillation proximity microtiter plates (FlashPlates) and incubated at 4° C. overnight. Incorporation of radioactivity was determined by scintillation counting.

### FORMULATION EXAMPLES

The following are representative pharmaceutical formulations containing a compound of Formula (A).

### Formulation Example 1

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| Ingredient | Quantity per capsule, mg |
|---|---|
| compound of this invention | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4

### Injectable Formulation

The following ingredients are mixed to form an injectable formulation.

| Ingredient | Amount |
|---|---|
| compound of this invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| Compound of the invention | 500 mg |
| Witepsol ® H-15 | balance |

1. A compound, tautomer, or stereoisomer of Formula (A) or pharmaceutically acceptable salts thereof: 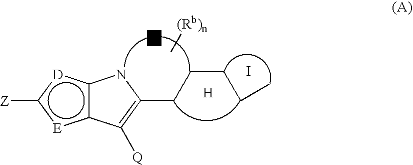
wherein:
ring H and ring I are independently an optionally substituted 6 member aryl or an optionally substituted 5 or 6 member heteroaryl having one, two, or three ring heteroatoms independently selected from the group consisting of N, NH, N-oxide, O, or S;
T is C₁ to C₅ alkylene wherein one or two —CH₂— groups are optionally replaced with —NR^{c}—, —S—, or —O— and optionally two —CH₂— groups together form a double bond provided that T does not contain an —O—O—, —S—O—, or —S—S— group;
Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and
one of D or E is C—R^{a} and the other of D or E is S; or D is CH and E is —CH═CH— such that Z, D, E and the atoms to which they are attached together form a fused 6-member ring with the remainder of the molecule: 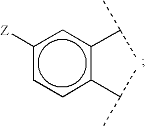
R^{a} is independently selected from the group consisting of hydrogen, alkyl, and substituted alkyl;
R^{b} is selected from the group consisting of halo, acyl, acylamino, alkyl, substituted alkyl, carboxy ester, hydroxy, and ═O;
n is 0, 1, or 2;
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

2. A compound, tautomer, or stereoisomer of claim 1 having Formula (I) or (II) or pharmaceutically acceptable salts thereof: 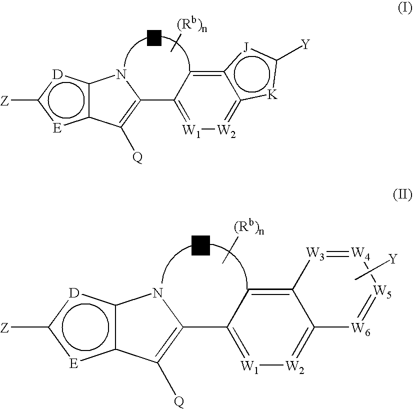
wherein:
Y is selected from the group consisting of hydrogen, alkyl, substituted alkyl, alkoxy, substituted alkoxy, amino, substituted amino, halo, hydroxy, nitro, aryl, heteroaryl, substituted aryl, and substituted heteroaryl;
J and K are independently selected from the group consisting of N, NH, CX, and N-oxide provided that J and K are not both CX;
W₁, W₂, W₃, W₄, W₅, and W₆ are independently selected from the group consisting of N, N-oxide, or CX, provided that no more than one of J, K, and W₁-W₆ is an N-oxide and provided that one of W₄ or W₅ is C—Y;
each X is independently selected from the group consisting of hydrogen, alkyl, substituted alkyl, alkoxy, substituted alkoxy, amino, substituted amino, halo, hydroxy, and nitro;
T is C₁ to C₅ alkylene wherein one or two —CH₂— groups are optionally replaced with —NR^{c}—, —S—, or —O— and optionally two —CH₂— groups together form a double bond provided that T does not contain an —O—O—, —S—O—, or —S—S— group;
Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and
one of D or E is C—R^{c} and the other of D or E is S; or D is CH and E is —CH═CH— such that Z, D, E and the atoms to which they are attached together form a fused 6-member ring with the remainder of the molecule: 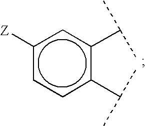
R^{a} and R^{c} are independently selected from the group consisting of hydrogen, alkyl, and substituted alkyl;
R^{b} is selected from the group consisting of halo, acyl, acylamino, alkyl, substituted alkyl, carboxy ester, hydroxy, and ═O;
n is 0, 1, or 2;
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

3. A compound of claim 2 wherein T is selected from the group consisting of —CH₂CH₂CH₂—, —CH₂CH═CH—, —CH₂CH₂CH₂CH₂—, —CH₂NR^{c}CH₂— and —CH₂CH₂NR^{c}CH₂—.

4. A compound of claim 2 having Formula (Ia) or (IIa) 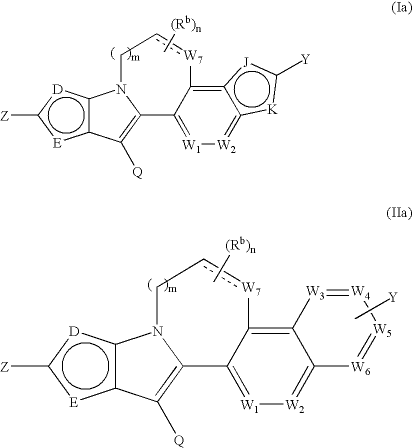
wherein W₇ is selected from the group consisting of CH, CH₂, NR^{c}, and O; m is 0, 1, or 2; the line [symbol] represents a single bond when W₇ is N or CH₂ or a double bond when W₇ is CH; and
Z, D, E, Q, R^{b}, R^{c}, n, J, K, W₁, W₂, W₃, W₄, W₅, W₆, and Y are previous defined.

5. A compound of claim 4 having Formula (Ib) or (IIb) 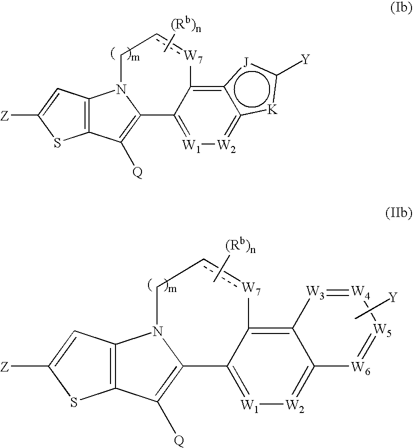
wherein Z, Q, R^{b}, n, m, J, K, W₁, W₂, W₃, W₄, W₅, W₆, W₇ and Y are previous defined.

6. A compound of claim 4 having Formula (Ic) or (IIc) 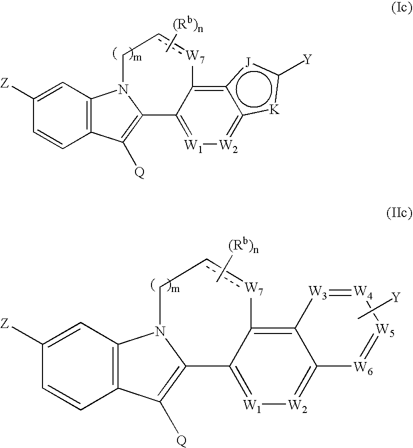
wherein Z, Q, R^{b}, n, m, J, K, W₁, W₂, W₃, W₄, W₅, W₆, W₇ and Y are previous defined.

7. A compound of claim 4 having Formula (Id) or (IId) 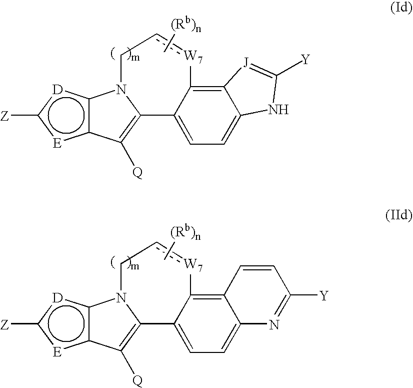
wherein J is CX or N, and Z, D, E, Q, R^{b}, n, m, W₇, Y are previous defined.

8. A compound of claim 7 having Formula (Ie) or (IIe) 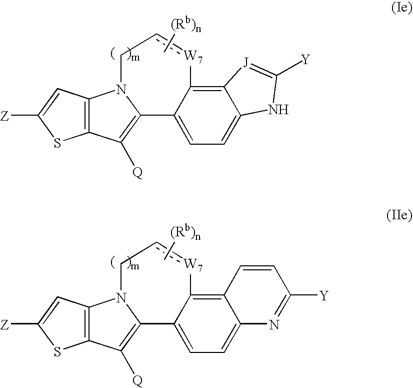
wherein J is CX or N, and Z, Q, R^{b}, n, m, W₇, Y are previous defined.

9. A compound of claim 7 having Formula (If) or (IIf) 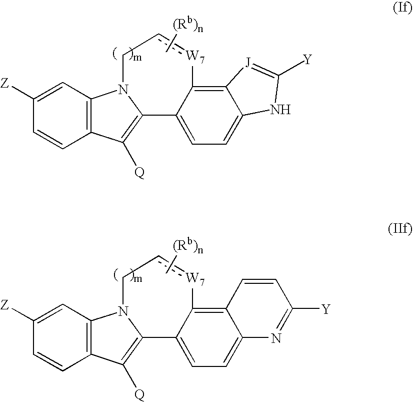
wherein J is CX or N, and Z, Q, R^{b}, n, m, W₇, Y are previous defined.

10. A compound of claim 2 wherein J is CH.

11. A compound of claim 2 wherein J is N.

12. A compound, tautomer, or stereoisomer of claim 1 having Formula (III) or (IV) or pharmaceutically acceptable salts thereof: 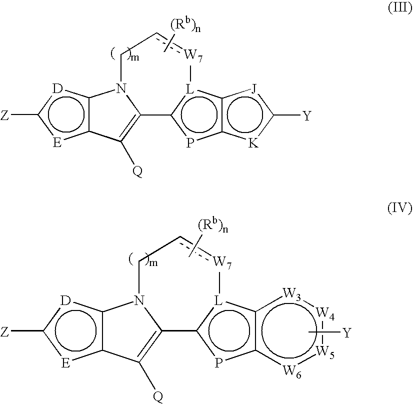
wherein:
Y is selected from the group consisting of hydrogen, alkyl, substituted alkyl, alkoxy, substituted alkoxy, amino, substituted amino, halo, hydroxy, nitro, aryl, heteroaryl, substituted aryl, and substituted heteroaryl;
J and K are independently selected from the group consisting of N, NH, CX, and N-oxide provided that J and K are not both CX;
when L is C, P is NH;
when L is N, P is N or CX and W₇ is CH or CH₂;
W₃, W₄, W₅, and W₆, are independently selected from the group consisting of N,N-oxide, or CX, provided that no more than one of J, K, and W₃-W₆ is an N-oxide and provided that one of W₄ or W₅ is C—Y;
m is 0, 1, or 2;
the line [symbol] represents a single bond when W₇ is N or CH₂ or a double bond when W₇ is CH;
W₇ is selected from the group consisting of CH, CH₂, and NR^{c};
each X is independently selected from the group consisting of hydrogen, alkyl, substituted alkyl, alkoxy, substituted alkoxy, amino, substituted amino, halo, hydroxy, and nitro;
Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl;
one of D or E is C—R^{a} and the other of D or E is S; or D is CH and E is —CH═CH— such that Z, D, E and the atoms to which they are attached together form a fused 6-member ring with the remainder of the molecule: 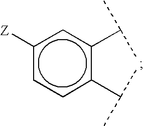
R^{a} and R^{c} are independently selected from the group consisting of hydrogen, alkyl, and substituted alkyl;
R^{b} is selected from the group consisting of halo, acyl, acylamino, alkyl, substituted alkyl, carboxy ester, hydroxy, and ═O;
n is 0, 1, or 2; and
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and
R⁹ together with the nitrogen atom pendent thereto, form a heterocyclic, a substituted heterocyclic, a heteroaryl or a substituted heteroaryl ring group;
(c) —C(X³)NR²S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²², and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R² are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R² to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

13. A compound, tautomer, or stereoisomer of claim 12 having Formula (IVa) or pharmaceutically acceptable salts thereof: 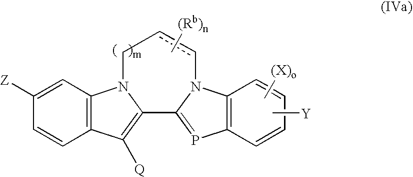
wherein Z, Q, R^{b}, n, m, X, and Y are previous defined, the line [symbol] represents a single bond or double bond, P is N or CH, and o is 0, 1, 2, or 3.

14. A compound of claim 13 wherein P is CH.

15. A compound of claim 1 wherein Z is carboxy, carboxy ester, carboxylic acid isostere, —C(O)NR⁸R⁹, or —C(O)NHS(O)₂R⁴, wherein R⁸ and R⁹ are as defined in claim 1 and R⁴ is alkyl or aryl.

16. A compound of claim 15 wherein Z is carboxy, methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, cyclopropylsulfonylamino, or phenylsulfonylaminocarbonyl.

17. A compound of claim 16 wherein Z is carboxy.

18. A compound of claim 1 wherein Z is selected from the group consisting of

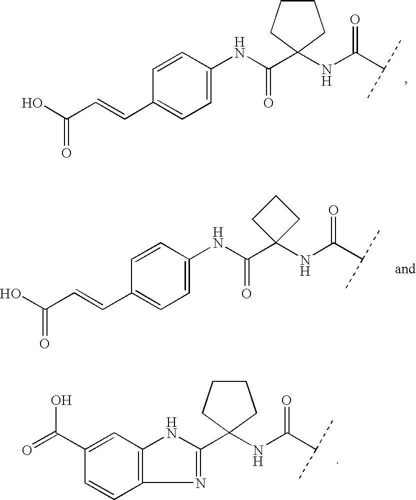

19. A compound of claim 1 wherein Q is cycloalkyl or substituted cycloalkyl.

20. A compound of claim 19 wherein Q is cyclohexyl or substituted cyclohexyl.

21. A compound of claim 20 wherein Q is 2-fluorocyclohexyl.

22. A compound of claim 2 or 12 wherein Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized.

23. A compound of claim 22 wherein Y is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphen-2-yl, 4-methoxybiphen-2-yl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

24. A compound of claim 22 wherein said substituted phenyl is substituted with one to three substitutents selected from the group consisting of halo, heteroaryl, hydroxy, nitro, cyano, alkyl, substituted alkyl, alkenyl, alkoxy, substituted alkoxy, acyl, acylamino, aminoacyl, amino, substituted amino, carboxy, and carboxy ester.

25. A compound of claim 22 wherein Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl.

26. A compound of claim 25 wherein Y is substituted with one to three substitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester.

27. A compound of claim 26 wherein Y is 2,4-dimethylthiazol-5-yl.

28. A compound of claim 1 wherein n is 1 and R^{b} is oxo.

29. A compound of claim 1 wherein n is 2 and both R^{b} are hydroxy.

30. A compound of claim 1 wherein R^{b} is —C(O)NR¹²R¹³ wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

31. A compound of claim 30 wherein R¹² and R¹³ together form a morpholino ring.

32. A compound of claim 2 wherein T is —CH₂CH═CH—.

33. A compound of claim 2 wherein T is —CH₂CH₂CH₂—.

34. A compound of claim 4 wherein m is 2.

35. A compound of claim 4 wherein m is 1.

36. A compound of claim 4 wherein W₇ is O.

37. A compound of claim 4 wherein W₇ is NR^{c}.

38. A compound of claim 1 wherein R^{c} is hydrogen.

39. A compound of claim 1 wherein R^{c} is alkyl substituted with heterocyclyl or substituted heterocyclyl.

40. A compound of claim 1 wherein R^{c} is —C(O)O(alkyl).

41. A compound of claim 1 wherein R^{c} is —CH₂C(O)NR¹²R¹³ wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

42. A compound of claim 41 wherein at least one of R¹² or R¹³ is alkyl, substituted alkyl, or heteroaryl.

43. A compound of claim 41 wherein at least one of R¹² or R¹³ is methyl, carboxymethyl, 2-hydroxyethyl, 2-morpholin-4-ylethyl, or tetrazoyl-5-yl.

44. A compound of claim 41 wherein R¹² and R¹³ and the nitrogen atom to which they are attached form a substituted or unsubstituted heterocyclic ring.

45. A compound of claim 41 wherein R¹² and R¹³ and the nitrogen atom to which they are attached form a substituted or unsubstituted morpholino, substituted or unsubstituted piperidinyl, or a substituted or unsubstituted pyrrolidinyl ring.

46. A compound of claim 45 wherein said substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

47. A compound of claim 41 wherein R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-b enzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido)pyrrolidin-3-yl]amino, N-[4-(N′-(4-(acrylic acid)-phenyl)carboxamido)piperidin-4-yl]amino, 2-(N,N-dimethylamino)eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, (1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N-(N′,N′-dimethylaminoacetyl)-amino, N-methyl-N-phenyl-amino, N-methyl-N-isopropyl-amino, N-methyl-N-(N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino)eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino)-piperidin-1-yl, 3-(N,N-dimethylamino)-methylpiperidin-1-yl, 2-(2-(N,N-dimethylamino)-eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl)-pyrrolidin-1-yl, 3-(N′,N′-dimethylamino)-pyrrolidin-1-yl, 3-(N′,N′-diethylamino)-pyrrolidin-1-yl, 3-(pyridin-3-yl)-pyrrolidin-1-yl, 2-pyridin-4-ylpyrrolidin-1-yl, piperazin-1-yl, 4-methylpiperazinyl, 4-(carboxymethyl)-piperazin-1-yl, 4-(2-hydroxyeth-1-yl)piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, and 4-(6-methoxypyridin-2-yl)piperazin-1-yl.

48. A compound, tautomer, or stereoisomer of claim 1 selected from Table 1 or pharmaceutically acceptable salts thereof.

49. A pharmaceutical composition comprising a pharmaceutically acceptable carrier and a therapeutically effective amount of a compound of claim 1 or 48.

50. A method for treating a viral infection in a mammal mediated at least in part by a virus in the Flaviviridae family of viruses, comprising administering to said mammal a composition of claim 49.

51. The method of claim 50 wherein the viral infection is mediated by hepatitis C virus.

52. The method of claim 50 in combination with a therapeutically effective amount of one or more agents active against hepatitis C virus.

53. The method of claim 52 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.
